(19) United States
(12) Patent Application Publication
(10) Pub. No.: US 2026/0266828 A1
(43) Pub. Date: Sep. 10, 2026

(54) BIOMARKERS FOR PREDICTING CANCER TREATMENT EFFICACY

(71) Applicant: Arcus Biosciences, Inc., Hayward, CA (US)

(72) Inventors: Daniel M. DIRENZO, Menlo Park, CA (US); Amy Grace DUPAGE, Kensington, CA (US); Kelsey Sivick GAUTHIER, Alameda, CA (US); Juan Carlos JAEN, Burlingame, CA (US); Salema JAFRI, Danville, CA (US); Omar KABBARAH, Danville, CA (US); Angelo Jason Mirizzi KAPLAN, San Jose, CA (US); Dana PIOVESAN, North Melbourne (AU); Rebecca RAY, Walnut Creek, CA (US); Lisa C. SEITZ, Oakland, CA (US); Matthew J. WALTERS, Pacifica, CA (US); Jonathan Michael YINGLING, Livermore, CA (US)

(21) Appl. No.: 19/489,212

(22) PCT Filed: May 31, 2024
(86) PCT No.: PCT/US2024/032049

§ 371 Date: Dec. 1, 2025

Related U.S. Application Data
(60) Provisional application No. 63/470,660, filed on Jun. 2, 2023.
(60) Provisional application No. 63/539,295, filed on Sep. 19, 2023.
(60) Provisional application No. 63/649,808, filed on May 20, 2024.

(51) Int. Cl. G01N 33/575; A61K 39/00; A61K 45/06; A61P 35/00; C07K 16/28; G01N 33/5752; G01N 33/5753

(52) U.S. Cl. CPC G01N 33/5759; A61K 45/06; A61P 35/00; C07K 16/28; C07K 16/2827; G01N 33/5752; G01N 33/5753; G01N 33/57585; A61K 2039/505; G01N 2333/522; G01N 2333/5412; G01N 2333/57; G01N 2333/70532; G01N 2333/70596; G01N 2474/20

(57) ABSTRACT

Provided herein are biomarkers useful in the identification of a patient for a therapy comprising an immune checkpoint inhibitor, and methods of using the same. In particular, the present disclosure provides technologies for the treatment and prognostication of cancer in a patient utilizing biomarker expression levels in a biological sample obtained from the patient, as well as technologies for determining the likelihood that the patient will respond to a therapy comprising an immune checkpoint inhibitor.

### CROSS REFERENCE TO RELATED APPLICATIONS

This application claims the benefit under 35 U.S.C. § 119(e) to U.S. Provisional Application No. 63/470,660, filed Jun. 2, 2023, 63/539,295 filed Sep. 19, 2023 and 63/649,808 filed May 20, 2024, all of which are incorporated by reference in their entirety.

### BACKGROUND

The following discussion is provided to aid the reader in understanding the disclosure and is not admitted to describe or constitute prior art thereto.

Cancers continue to cause a significant burden throughout the world. Lung cancer is the most common form of cancer and leading cause of cancer death worldwide. In 2018, over two million cases of lung cancer were diagnosed, and lung cancer was responsible for 1.76 million deaths worldwide. Of the histologies, non-small cell lung cancer (NSCLC) represents approximately 80% of all lung cancer diagnoses. The vast majority of NSCLC presents at an advanced stage. Survival for patients with metastatic NSCLC is poor, with a 5-year survival rate of approximately 4%.

Globally, gastric cancer and esophageal cancer are the fifth and seventh most frequently diagnosed cancers, respectively. While the incidence of these malignancies varies across the world, survival rates for patients with locally advanced unresectable and/or metastatic disease remain low regardless of geography. Gastric cancer is the third highest cause of cancer death worldwide, while esophageal cancer is the sixth highest cause of cancer death globally. Further, in the United States, rates of esophageal adenocarcinoma have been increasing, the incidence of gastric cancer may be increasing in people younger than age 50, and the 5-year survival rate in patients with metastatic esophageal or gastric cancer is approximately 5 percent (National Cancer Institute SEER Program, 2021a; National Cancer Institute SEER Program, 2021b).

Immunotherapies are a promising class of drugs for treating cancer. Immunotherapy treatments targeting the programmed death receptor 1 (PD-1), the programmed death ligand 1 (PD-L1), cytotoxic T-lymphocyte-associated protein 4 (CTLA-4), and lymphocyte-activation gene 3 (LAG-3) have received regulatory approval for various cancer indications and several new classes of immunotherapies targeting other immune checkpoint inhibitors, including but not limited to, lung cancer, head and neck cancer, gastric and esophageal cancer, bladder cancer and hematologic malignancies, are in clinical trials. Although recent advances in immunotherapy have or may alter the treatment paradigm for a number of cancers, many patients do not respond to immunotherapy or they eventually relapse. Resistance can develop through various mechanisms including the persistence of a resistant clone or alterations in the tumor microenvironment (TME). Moreover, the immune system as it interacts with the TME may not be simply solved by inhibiting one axis alone. Other regulators of a complex immune system may provide additional mechanisms of interest in advancing the treatment of cancers.

Thus, there remains a need for effective treatment regimens for therapies involving immunotherapies such as immune checkpoint inhibitors that deliver meaningful clinical efficacy and means for identifying patients and predicting which patients will respond.

### SUMMARY

The present disclosure provides biomarkers useful in numerous contexts, particularly regarding identifying a patient for treatment of a disease, particularly cancer, with a therapy comprising an immune checkpoint inhibitor.

In one aspect, the present disclosure provides uses of a biomarker to identify a patient for treatment with a therapy comprising an immune checkpoint inhibitor, wherein the patient has cancer and is identified for treatment with an immune checkpoint inhibitor when a sample obtained from the patient comprises a biomarker selected from: (a) a PD-L1 expression level greater than or equal to a PD-L1 reference level or lower than a PD-L1 reference level; (b) a CD155 expression level greater than or equal to a CD155 reference level or lower than a CD155 reference level; (c) a CD226 expression level greater than or equal to a CD226 reference level or lower than a CD226 reference level; (d) an adenosine pathway biomarker expression level greater than or equal to an adenosine pathway biomarker reference level or lower than an adenosine pathway biomarker reference level; (e) a CD73 expression level greater than or equal to a CD73 reference level or lower than a CD73 reference level; or (f) any combination of (a), (b), (c), (d), and (e). In some embodiments, the patient is identified for treatment with an immune checkpoint inhibitor when: (a) the PD-L1 expression level is greater than or equal to a PD-L1 reference level; (b) the CD155 expression level is greater than or equal to a CD155 reference level; (c) the CD226 expression level is greater than or equal to a CD226 reference level; (d) the adenosine pathway biomarker expression level is lower than an adenosine pathway biomarker reference level; (e) the CD73 expression level is lower than a CD73 reference level; or (f) any combination of (a), (b), (c) (d), and (e).

In one aspect, the present disclosure provides uses of a biomarker to determine a prognosis for a patient who has cancer, wherein the biomarker is selected from PD-L1, CD155, CD226, CD73, an adenosine pathway biomarker, and any combination thereof, wherein the patient is determined to have a favorable prognosis when a sample obtained from the patient comprises: (a) a PD-L1 expression level greater than or equal to a PD-L1 reference level or lower than a PD-L1 reference level; (b) a CD155 expression level greater than or equal to a CD155 reference level or lower than a CD155 reference level; (c) a CD226 expression level greater than or equal to a CD226 reference level or lower than a CD226 reference level; (d) an adenosine pathway biomarker expression level greater than or equal to an adenosine pathway biomarker reference level or lower than an adenosine pathway biomarker reference level; (e) a CD73 expression level greater than or equal to a CD73 reference level or lower than a CD73 reference level; or (f) any combination of (a), (b), (c), (d), and (e).

In one aspect, the present disclosure provides a method for treating cancer in a patient, comprising administering to the patient a therapy comprising an immune checkpoint inhibitor when a sample obtained from the patient comprises: (a) a PD-L1 expression level greater than or equal to a PD-L1 reference level or lower than a PD-L1 reference level; (b) a CD155 expression level greater than or equal to a CD155 reference level or lower than a CD155 reference level; (c) a CD226 expression level greater than or equal to a CD226 reference level or lower than a CD226 reference level; (d) an adenosine pathway biomarker expression level greater than or equal to an adenosine pathway biomarker reference level or lower than an adenosine pathway biomarker reference level; (e) a CD73 expression level greater than or equal to a CD73 reference level or lower than a CD73 reference level; or (f) any combination of (a), (b), (c) (d), and (e). In some embodiments, the therapy is administered to the patient when: (a) the PD-L1 expression level is greater than or equal to a PD-L1 reference level; (b) the CD155 expression level is greater than or equal to a CD155 reference level; (c) the CD226 expression level is greater than or equal to a CD226 reference level; (d) the adenosine pathway biomarker expression level is lower than an adenosine pathway biomarker reference level; or (e) the CD73 expression level is lower than a CD73 reference level; (f) any combination of (a), (b), (c), (d), and (e).

In one aspect, the present disclosure provides a method for identifying a patient with cancer for treatment with a therapy comprising an immune checkpoint inhibitor, comprising: (a) measuring in a sample obtained from a patient a level of one or more of PD-L1, CD155, CD226, an adenosine pathway biomarker, and CD73; (b) comparing the level measured in (a) to a respective reference level; and (c) identifying the patient for treatment with the therapy when (i) the measured PD-L1 level is greater than or equal to a PD-L1 reference level or lower than a PD-L1 reference level; (ii) the measured CD155 level is greater than or equal to a CD155 reference level or lower than a CD155 reference level; (iii) the measured CD226 level is greater than or equal to a CD226 reference level or lower than a CD226 reference level; (iv) the measured adenosine pathway biomarker level is greater than or equal to an adenosine pathway biomarker reference level or lower than an adenosine pathway biomarker reference level; (v) the measured CD73 level greater than or equal to a CD73 reference level or lower than a CD73 reference level; of (vi) any combination of (i), (ii), (iii), (iv), and (v). In some embodiments, the patient is identified for treatment when: (i) the measured PD-L1 level is greater than or equal to a PD-L1 reference level; (ii) the measured CD155 level is greater than or equal to a CD155 reference level; (iii) the measured CD226 level is greater than or equal to a CD226 reference level; (iv) the measured adenosine pathway biomarker level is lower than an adenosine pathway biomarker reference level; (v) the measured CD73 level is lower than a CD73 reference level; or (vi) any combination of (i), (ii), (iii), (iv), and (v).

In any of the foregoing aspects and embodiments, the therapy can be a monotherapy or a combination therapy. In some embodiments, the therapy includes an anti-PD-(L)1 antibody, an anti-TIGIT antibody, optionally wherein the anti-TIGIT antibody is domvanalimab, and/or an ATP-adenosine axis-targeting agent. In some embodiments, the combination therapy includes chemotherapy. In some embodiments, the chemotherapy comprises a platinum-containing agent.

The methods provided herein are useful in the treatment of cancer, including gastrointestinal cancer, genitourinary cancer, gynecological cancer, and lung cancer.

Both the foregoing summary and the following description of the drawings and detailed description are exemplary and explanatory. They are intended to provide further details of the disclosure but are not to be construed as limiting. Other objects, advantages, and novel features will be readily apparent to those skilled in the art from the following detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF THE DRAWINGS

The patent or application file contains at least one drawing executed in color. Copies of this patent or patent application with color drawing(s) will be provided by the Office by request and payment of the necessary fee.

FIG. 1A, FIG. 1B, FIG. 1C and FIG. 1D are graphs depicting that TIGIT, PD-1, CD226, and associated ligands are expressed on broad cell populations in human NSCLC TIL suspensions. FIG. 1A depicts the frequency of T_{reg} (CD4⁺FoxP3+), CD4⁺ T (CD4⁺FoxP3⁻), CD8⁺ T, CD3⁻CD19⁺ B, CD3⁻CD56⁺ NK, CD14⁺ and/or CD16⁺ monocyte, SSC^{hi} myeloid/macrophage, and CD45⁻ cancer or stromal subsets in NSCLC tumor-infiltrating lymphocyte (TIL) suspensions. FIG. 1B depicts the frequency (circles, left y-axis) and geometric mean fluorescence intensity (gMFI) (triangles, right y-axis) of TIGIT (top), PD-1 (middle), and CD226 (bottom) in CD4⁺, T_{reg}, and CD8⁺ T subsets. FIG. 1C depicts the frequency of PD-1⁺TIGIT⁺CD226⁺ triple positive cells in CD4⁺, T_{reg}, and CD8⁺ T subsets.

FIG. 1D depicts the frequency (circles, left y-axis) and gMFI (triangles, right y-axis) of CD155 (top) or PD-L1 (bottom) in subsets from (FIG. 1A). PD-L1 gMFI is plotted as a fold change (FC) relative to isotype (iso) because baseline expression was dramatically different between subsets. Data from n=6-10 NSCLC subjects were compiled from two independent experiments. Symbols represent individual subjects. Bars and error represent median±range. Ordinary one-way ANOVA with Tukey's multiple comparisons test, *p<0.05, **p<0.01, ***p<0.001, 894 ****p<0.0001. See gating strategy in FIG. 7A-FIG. 7C.

FIG. 2A, FIG. 2B, FIG. 2C, FIG. 2D, FIG. 2E, FIG. 2F and FIG. 2G show that TIGIT and PDCD1, but not CD226, are expressed on pre-exhausted Tₚₑₓ cells in scRNA-seq datasets, whereas TIGIT, PD-1, and CD226 are detected at the protein level in Tₑₓ subsets in commercially sourced human NCSLC TIL suspensions. TIGIT, PDCD1, and CD226 gene expression were evaluated on Tₚₑₓ populations in two publicly available human NSCLC scRNA-seq datasets. Tₚₑₓ were identified by applying filters to single-cell clusters in the following order: medium to high expression of PD-1 (PCDC1), negative expression of TIM-3 (HAVRC2), and positive expression of granzyme K (GZMK). FIG. 2A depicts UMAP plots mapping Tₚₑₓ cells in the Guo et al. (top left), and Gueguen et al. datasets (top right), and percentage of Tₚₑₓ to total CD8⁺ T cells in each dataset (bottom). Box (25^{th} to 75^{th} percentile) and whisker (mix to max) plots with symbols representing each NSCLC subject. n=11-14.

FIG. 2B depicts UMAP plots mapping PDCD1 (top), TIGIT (middle), CD226 (bottom) expression to total CD8⁺ T cells in each dataset as indicated. Shading highlights Tₚₑₓ cells.

FIG. 2C depicts a representative pseudocolor plot (top) and frequency of Tₚₑₓ (TCF-1⁺TIM-3⁻) and Tₜₑₓ (TCF-1⁻TIM-3+) containing populations in the CD8⁺ T subset of the NSCLC TIL suspensions. FIG. 2D depicts representative histograms for PD-1, TIGIT, CD39, CD226, GzmK, GzmB, and CD103 in TCF-1⁺TIM-3⁻ and TCF-1⁻TIM-3⁺ populations.

FIG. 2E depicts representative dot plots of TIGIT, CD226, and PD-1 expression (top) and frequency of PD-1⁺TIGIT⁺CD226⁺ triple positive cells in TCF-1⁺TIM-3 and TCF-1⁻TIM-3⁺ populations (bottom). FIG. 2F depicts an alternative method for identifying tissue resident (CD103⁺) or circulating (CD103⁻) functional (PD-1⁻), pre-exhausted Tₚₑₓ (PD-1+), and terminally differentiated/dysfunctional Tₜₑₓ (PD-1^{hi}) containing CD8⁺ T cell subsets in NSCLC TIL suspensions (top); and frequency of six sub-populations in the total CD8⁺ T subset (bottom). FIG. 2G depicts representative histograms (top left) and plotted gMFI (top right) for TIGIT and CD226 on six populations from FIG. 2F; and frequency (bottom) of TIGIT⁺CD226⁺ double positive cells in six populations from FIG. 2F. In FIG. 2C-FIG. 2G, n=5-9 NSCLC subjects. Symbols represent individual subjects and lines connect the same subject. Bars and error represent median±range. Ordinary one-way ANOVA with Tukey's multiple comparisons test, *p<0.05, ***p<0.001, ****p<0.0001. Only data with >100 events per population are shown. See gating strategy in FIG. 7A-FIG. 7C. See also FIG. 8.

FIG. 3A, FIG. 3B, FIG. 3C and FIG. 3D show that TIGIT, PD-1, and CD226 are co-expressed in MC38 mouse tumors and tdLN. MC38 tumors (50-150 mm³) and inguinal tdLNs were interrogated for expression of TIGIT, PD-1, CD226, CD155, and PD-L1 by flow cytometry. n=8-10. Symbols represent individual mice. Bars and error represent median±range. Only data with >100 events per population are shown. Representative data for at least three independent studies are shown. FIG. 3A depicts the frequency of Tₚₑₓ (TCF-1⁺TIM-3⁻) and Tₜₑₓ (TCF-1⁻TIM-3+) containing populations in the tumor and tdLN. FIG. 3B depicts the frequency of TIGIT⁺, PD-1⁺, and CD226⁺ single positive and TIGIT⁺PD-1⁺CD226⁺ triple positive (+++) cells in tumor (Tu) TCF-1⁻TIM-3⁺, Tu TCF-1⁺ TIM-3⁻, and tdLN TCF-1⁺TIM-3⁻, and populations (left), and representative histograms in a tumor sample (right). FIG. 3C depicts the frequency of TIGIT⁺, PD-1⁺, CD226⁺, and triple positive (+++) cells in tumor T_{reg}, CD4⁺ T, CD8⁺ T, and NK subsets. FIG. 3D depicts the frequency of CD155 and PD-L1 in CD11b+Ly6C⁺ monocytes (mono), CD11b⁺F4/80⁺ TAM (in tumor) or macrophage (mac) in tdLN, CD11b⁺CD11c⁺MHCII⁺ myeloid dendritic cells (mDC), CD11b⁻CD11c⁺MHCII⁺CD11b⁺Ly6C⁺ conventional DC (cDC), CD11b⁺Ly6G⁺ neutrophils (ne), and CD45⁻ cancer and stromal subsets (left). CD45⁻ and ne populations were not present in interrogatable numbers in the tdLN. Representative histograms in a tumor sample are shown on the right.

FIG. 4A, FIG. 4B, FIG. 4C, FIG. 4D, FIG. 4E, and FIG. 4F show that in combination with anti-PD-1, Fcₛ anti-TIGIT promotes anti-tumor immunity associated with an increase in tumor-specific T cells in the TME and Tₚₑₓ differentiation that is dependent on LN egress. FIG. 4A, FIG. 4B graphically depict data from mice with established MC38 tumors (70-80 mm³) dosed with an optimized dosing regimen of 10 mg/kg αPD-1 (squares on x-axis) or 10 mg/kg αPD-1+10 mg/kg αTIG Fcₛ (inverted triangles on x-axis) every 5 days (Q5D) throughout the course of the study. FIG. 4A depicts average tumor volume growth curves where symbols and error represent the mean±SEM (top), and individual tumor volume growth curves with number of CRs per treatment group (middle and bottom). n=15/group. In all graphs, the y-axis is tumor volume (mm³) and the x-axis is days after implant. General linear model with baseline tumor volume and duration of treatment as covariates; Benjamini-Hochberg adjustment for multiple comparisons, ****p<0.0001. FIG. 4B depicts a Kaplan-Maier survival curve (right). Cox proportional hazards survival analysis, ****p<0.0001. Representative data of at least three independent experiments. FIG. 4C, FIG. 4D, FIG. 4E, and FIG. 4F depict data from MC38 tumor-bearing mice with established tumors (~60 mm³) treated with vehicle or 0.25 mg/kg FTY720 daily by oral gavage starting one day prior to Q5D antibody treatment. Three days after the first antibody dose, tumors were collected for the flow cytometric analyses shown in FIG. 4C, FIG. 4D, and FIG. 4E. FIG. 4C depicts representative dot plots identifying tumor-specific p15e⁺CD8⁺ T cells (left). FIG. 4D depicts frequency of p15e⁺ T cells in the tumoral CD8⁺ T subset (top) and number of p15e⁺ T cells per mg of tumor relative to the mean of the isotype+vehicle group (bottom). Data were compiled from three independent experiments each with n=8/group. Kruskal-Wallis test with Dunn's multiple comparisons test, *p<0.05, **p<0.01, ****p<0.0001. FIG. 4E depicts a representative dot plot showing the differentiation states of exhausted CD8⁺ T cells in the tumor based on TCF-1/CD69 staining (top), and frequency of resident Tₚₑₓ (rTₚₑₓ), circulating Tpcx (cTpₑₓ), intermediate effector (Int), and terminally differentiated (Tₜcx) p15E⁺ CD8⁺ T cells relative to the mean of the isotype+vehicle group (bottom, legend below graph). Data were compiled from three independent experiments each with n=8/group. Two-way ANOVA with Tukey's multiple comparisons test, *p<0.05, **p<0.01, ***p<0.001, ****p<0.0001. FIG. 4F depicts average tumor volume growth curves where symbols and error represent the mean±SEM (top) and individual tumor volume growth curves with number of CRs per treatment group (bottom). n=10/group. In all graphs, the y-axis is tumor volume (mm³) and the x-axis is days after implant. General linear model with baseline tumor volume and duration of treatment as covariates; Tukey's adjustment for multiple comparisons, *p<0.05, **p<0.01, ***p<0.001, ****p<0.0001. Representative data for four independent experiments are shown. iso, isotype control; FC, fold change. See gating strategy in FIG. 7D-FIG. 7E. See also FIG. 10.

FIG. 5A, FIG. 5B, and FIG. 5C show that Lethal OXA chemotherapy concentrations upregulate PD-L1 and CD155 on MC38 cells in vitro. FIG. 5A depicts cell viability (as measured by ATP) of MC38 cells treated with OXA at indicated concentrations for 72 h. The shaded area identifies non-cytotoxic concentrations. Lines and error represent the mean±standard deviation (SD) of biological triplicates. FIG. 1B depicts gMFI of CD155 (circles, left y-axis) and PD-L1 (squares, right y-axis) on MC38 cells treated with OXA at indicated concentrations for 22 h (top), and representative histograms for CD155 and PD-L1 on MC38 cells (bottom). FIG. 5C depicts gMFI of CD155 (circles, left y-axis) and PD-L1 (squares, right y-axis) on MC38 cells treated with OXA at indicated (non-cytotoxic) concentrations for 72 h. In FIG. 5B and FIG. 5C, lines and error represent the mean±SD of biological duplicates. Gray shading indicates experimentally determined non-cytotoxic OXA concentration range (0.001-0.03 μM), whereas horizontal dotted lines indicate the baseline staining for PBS treated cells (gMFI fold change relative to isotype control). Representative data for three independent experiments are shown. h, hours; iso, isotype control; FC, fold-change; OXA, oxaliplatin.

FIG. 6A, FIG. 6B, FIG. 6C, and FIG. 6D show that Fcₛ anti-TIGIT combines favorably with chemotherapy in the MC38 mouse model. FIG. 6A and FIG. 6B depict data from mice with established MC38 tumors (80-95 mm³) dosed with 10 mg/kg OXA (with dosing every seven days, Q7D, gray triangles on x-axis) alone or in combination with 10 mg/kg αPD-1 or 10 mg/kg αPD-1+10 mg/kg αTIG Fcₛ Q5D. Antibody treatments commenced three days after the first OXA dose. Average tumor volume growth curves where symbols and error represent the mean±SEM (FIG. 6A, top) and individual tumor volume growth curves with number of CRs per treatment group (FIG. 6B). n=14-15/group. General linear model with baseline tumor volume and duration of treatment as covariates; Benjamini-Hochberg adjustment for multiple comparisons, *p<0.05. Kaplain-Maier survival curve (FIG. 6A, bottom). Cox proportional hazards survival analysis with pairwise comparisons using Benjamini-Hochberg adjustment for multiple comparisons. *p<0.05, **p<0.01, ****p<0.0001. Representative data for four independent experiments are shown. FIG. 6C depicts data from mice with established MC38 tumors dosed as indicated in FIG. 6A with additional treatment arm of αTIG Fcₛ+OXA. Average tumor volume growth curves are shown, where symbols and error represent the mean±SEM. n=15/group. General linear model with baseline tumor volume and duration of treatment as covariates; Benjamini-Hochberg adjustment for multiple comparisons, ns, not significant, ****p<0.0001. Representative data for two independent experiments are shown. FIG. 6D depicts data from three days after the OXA dose, when tumors were collected for flow cytometric analysis. Frequency of CD11b⁺Ly6C⁺ monocytes (mono) and CD11b⁺Ly6G⁺ neutrophils (ne) in the CD45⁺ subset (left) or frequency of TCF-1^{+/-} cells in the CD8⁺ T subset (right). n=8/group. Ordinary two-way ANOVA with Sidak's multiple comparisons test, *p<0.05, ****p<0.0001. Representative data for two independent experiments are shown. iso, isotype control; OXA, oxaliplatin. See gating strategy in FIG. 7D and FIG. 7E.

FIG. 7A depicts a flow cytometry gating strategy for identifying cell populations in human NSCLC cell suspensions. Shown is a representative NSCLC sample.

FIG. 7B depicts a continuation of the flow cytometry gating strategy depicted in FIG. 7A.

FIG. 7C depicts a continuation of the flow cytometry gating strategy depicted in FIG. 7B.

FIG. 7D depicts a flow cytometry gating strategy for identifying cell populations in mouse MC38 tumor and inguinal tdLN cell suspensions. Shown is a representative MC38 tumor sample.

FIG. 7E depicts a continuation of the flow cytometry gating strategy depicted in FIG. 7D.

FIG. 8A, FIG. 8B, FIG. 8C, FIG. 8D, FIG. 8E, and FIG. 8F provide additional characterization of circulating Tₚₑₓ cells identified in scRNAseq datasets, the relationship between CD226 and TIGIT mRNA and protein expression in CD8⁺ T cells, and additional phenotyping TIGIT, PD-1, and CD226 in exhausted T cell subsets in commercially sourced human NCSLC and gastro-esophageal (G-E) TIL suspensions. FIG. 8A depicts expression of additional markers in CD8⁺ T cells from the Guo et al. (left) and Guegen et al. (right) datasets. Shading highlights Tₚₑₓ cells. FIG. 8B depicts frequency of protein (left y-axis) and relative mRNA (right y-axis) expression of TIGIT (left) and CD226 (right) in isolated healthy human peripheral blood CD8⁺ T cells. Symbols represent individual subjects, and lines connect the same subject. n=6 healthy donors compiled from two independent experiments. FIG. 8C depicts the frequency of six CD8⁺ T sub-populations described in FIG. 2F in the total CD8⁺ T subset of G-E TIL. FIG. 8D depicts the frequency of GzmK (left) and CD39⁺TIM-3⁺ co-expression (right) in six CD8⁺ T sub-populations in G-E and NSCLC subjects. FIG. 8E depicts the frequency of TIGIT and CD226 in six CD8⁺ T sub-populations in NSCLC TIL suspensions. FIG. 8F depicts the frequency of TIGIT⁺CD226⁺ double positive cells in six CD8′ T sub-populations in G-E TIL suspensions. In FIG. 8C, FIG. 8D, and FIG. 8F, n=4 each of G-E and NSCLC subjects. In FIG. 8E, n=10 NSCLC subjects. Two-way ANOVA with Tukey's multiple comparisons test, *p<0.05, **p<0.01, ***p<0.001, ****p<0.0001. Symbols represent individual subjects. Only data with >100 events per population are shown. See also FIG. 2 and FIG. 7A-FIG. 7C.

FIG. 9A, FIG. 9B, FIG. 9C and FIG. 9D show additional MC38 tumor and tdLN flow cytometry phenotyping data. FIG. 9A depicts the frequency of cell subsets in MC38 tumors or tdLN. For the tumors, the frequency of CD45⁺ immune cells and CD45⁻ (cancer and stromal) cells in the live singlet population. CD8⁺ T (CD8), CD4⁺FoxP3⁺ (T_{reg}), CD4⁺FoxP3⁻ T (CD4), NK1.1⁺ (NK), CD11b⁺Ly6G⁺ neutrophils (ne), CD11b⁺Ly6C⁺ monocytes (mono), CD11b⁺F4/80⁺ macrophages (TAM in tumor, mac in tdLN), CD11b⁺CD11c⁺MHCII⁺ myeloid dendritic cells (mDC), CD11b⁻CD11c⁺MHCII⁺ conventional DC (cDC). FIG. 9B depicts the frequency of CD39 or GzmB in TCF-1⁺TIM-3⁻ and TCF-1⁺TIM-3⁺ subsets in the tumor. FIG. 9C depicts the frequency of TIGIT, PD-1, CD226, and TIGIT⁺PD-1⁺CD226⁺ triple positive (+++) cells in tdLN T_{reg}, CD4⁺, CD8⁺, and NK subsets. n=10. FIG. 9D depicts global changes in immune cell populations within the TME across treatment groups. Individual mouse data per group was concatenated and represented as a stacked bar graph displaying different tumor immune cell subsets. n=8 mice/group. See also FIG. 3 and FIG. 7D-FIG. 7E.

FIG. 10A, FIG. 10B, FIG. 10C, and FIG. 10D show additional in vivo MC38 phenotyping data. FIG. 10A depicts data from naïve C57BL/6 mice dosed by oral gavage with vehicle (V), 0.25, 1, or 3 mg/kg FTY720. Frequency of CD3⁺ of live singles was measured in the blood 24 h later as a pharmacodynamic readout; 0.25 mg/kg was selected for MOA studies. n=5/group. Ordinary one-way ANOVA with Dunnet's multiple comparisons test vs. vehicle control, p<0.0001. Representative data of two independent studies. FIG. 10B, FIG. 10C, and FIG. 10D show additional data from MC38 studies shown in FIG. 4. FIG. 10B depicts frequency of p15E⁺ T cells in the tdLN CD8⁺ T subset (left) and number of p15E′ T cells per mg of tdLN tissue relative to the mean of the isotype+vehicle group (right). Data were compiled from three independent experiments each with n=8/group. Kruskal-Wallis test with Dunn's multiple comparisons test, *p<0.05, **p<0.01. FIG. 10C depicts frequency of resident Tₚₑₓ (rTₚₑₓ), circulating T_{Pex} (cTₚₑₓ), intermediate effector (Int), and terminally differentiated (Tₜₑₓ) p15E⁻ T cells relative to the mean of the isotype+vehicle group. Data were compiled from three independent experiments each with n=8/group. Two-way ANOVA with Tukey's multiple comparisons test, *p<0.05, **p<0.01, ***p<0.001, ****p<0.0001. FIG. 10D depicts an analysis of data using TCF-1/TIM-3 gating to identify Tₚₑₓ and Tₜₑₓ containing populations in tumor tissue. Representative dot plot showing the differentiation states of exhausted CD8⁺ T cells in the tumor based on TCF-1/TIM-3 staining (top). Frequency of TCF-1⁺TIM-3⁻ and TCF-1⁻TIM-3⁺ T cells in the tumor p15E⁺CD8⁺ T subset relative to the mean of the isotype+vehicle group (bottom). Data were compiled from three independent experiments each with n=8/group. Two-way ANOVA with Tukey's multiple comparisons test, *p<0.05, **p<0.01, ***p<0.001. See also FIG. 4.

FIG. 11 is a Kaplan-Meier plot of 1L metastatic NSCLC patients (PD-L1 expression of TPS 0-100%) grouped by CD155 expression (high vs low). CD155 high (black line) is defined by CD155 gene expression equal to or greater than median CD155 expression. CD155 low (blue line) is defined by CD155 gene expression less than median CD155 expression.

FIG. 12A is a Kaplan-Meier plot of 1L metastatic NSCLC patients with PD-L1 expression of TPS <1% grouped by CD155 expression (high vs. low). CD155 high (black line) is defined by CD155 gene expression equal to or greater than median CD155 expression. CD155 low (blue line) is defined by CD155 gene expression less than median CD155 expression.

FIG. 12B is a Kaplan-Meier plot of 1L metastatic NSCLC patients with PD-L1 expression of TPS=1-49% grouped by CD155 expression (high vs. low). CD155 high (black line) is defined by CD155 gene expression equal to or greater than median CD155 expression. CD155 low (blue line) is defined by CD155 gene expression less than median CD155 expression.

FIG. 12C is a Kaplan-Meier plot of 1L metastatic NSCLC patients with PD-L1 expression of TPS ≥50% grouped by CD155 expression (high vs. low). CD155 high (black line) is defined by CD155 gene expression equal to or greater than median CD155 expression. CD155 low (blue line) is defined by CD155 gene expression less than median CD155 expression.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

The present disclosure is drawn to biomarkers useful in numerous contexts, particularly regarding identifying a patient for treatment of a disease, particularly cancer, with a therapy comprising an immune checkpoint inhibitor. Also provided are methods for determining a prognosis for a patient who has or who is yet to receive a therapy comprising an immune checkpoint inhibitor. The present disclosure is also directed to technologies for the treatment of a patient who has been identified as having cancer, comprising measuring the expression level of one or more biomarkers in a sample obtained from the patient, and administering to the patient a therapy comprising an immune checkpoint inhibitor when the sample obtained from the patient comprises a particular biomarker expression profile. The present disclosure is directed to the discovery that the expression profile of certain biomarkers, including but not limited to PD-L1, CD155, CD226, CD73, and/or an adenosine pathway biomarker, can be used to determine the likelihood that a patient will be responsive or non-responsive to a therapy comprising an immune checkpoint inhibitor. In the Examples described herein, it was surprisingly observed that certain biomarker expression profiles can be used to predict treatment efficacy for a therapy comprising an immune checkpoint inhibitor. Exemplary immune checkpoint inhibitors are PD-(L)1 antagonists (e.g., anti-PD-L1 antibodies) and TIGIT antagonists (e.g., anti-TIGIT antibodies).

### Definitions

Unless otherwise defined, all terms of art, notations and other scientific terms or terminology used herein are intended to have the meanings commonly understood by those of skill in the art to which this disclosure pertains.

The term “about” as used herein has its original meaning of approximately and is to provide literal support for the exact number that it precedes, as well as a number that is near to or approximately the number that the term precedes. In general, the term “about” refers to the usual error range for the respective value readily known to the skilled person in this technical field. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The “amount,” “level,” or “expression level,” used herein interchangeably, of a biomarker is a detectable level in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression levels can be measured by methods known to one skilled in the art and also disclosed herein. The expression level or amount of a biomarker (e.g., CD155, CD226, PD-L1, CD73) can be used to identify/characterize a cancer that may be likely to respond to, or benefit from, a particular therapy or to not experience clinical benefit.

The term “biomarker” as used herein refers to an indicator, e.g., predictive, diagnostic, and/or prognostic, which can be detected in a sample. The biomarker may serve as an indicator of a particular subtype of a disease or disorder characterized by certain, molecular, pathological, histological, and/or clinical features. Biomarkers include, but are not limited to, polypeptides, polynucleotides (e.g., DNA, and/or RNA), polynucleotide copy number alterations (e.g., DNA copy numbers), polypeptide and polynucleotide modifications (e.g., methylation, acetylation, oxidation, glycosylation), carbohydrates, and/or glycolipid-based molecular markers.

The terms “detecting” and “detection” are used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

The term “sample,” as used herein, refers to a composition that is obtained or derived from a subject that contains a biomarker that is to be identified and/or characterized, for example based on physical, biochemical, chemical and/or physiological characteristics. For example, the phrase “tumor sample,” “disease sample,” and variations thereof refers to any sample obtained from a subject of interest that would be expected or is known to contain the biomarker that is to be characterized. Other samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, stool, whole lymph nodes or lymph node biopsies, tissue from biopsies or resections, tumor biopsies, tumor lysates, and tissue culture medium, tissue extracts such as homogenized tissue, cellular extracts, and combinations thereof.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject. In yet another embodiment, a reference sample, reference cell, reference tissue, control sample, control cell, or control tissue is obtained from a healthy and/or non-diseased part of the body (e.g., tissues or cells) of a subject who is not the subject. In even another embodiment, a reference sample, reference cell, reference tissue, control sample, control cell, or control tissue is obtained from an untreated tissue and/or cell of the body of an individual who is not the subject.

A “tumor sample” refers to a sample comprising tumor cells. Typically a tumor sample obtained from a solid tumor (e.g., tissue from biopsies or resections) comprises tumor cells and other cells of the tumor microenvironment (e.g., immune cells, stromal cells, fibroblasts, etc.).

The term “positive cell fraction” is the percentage of viable cells showing positive staining for a protein of interest in one or more cellular location (e.g., membrane, cytoplasm) at any intensity in a sample, following staining of the sample, e.g., in an immunohistochemical (IHC) assay. A positive cell fraction may be reported for all cell types or a certain subtype or subtypes (e.g., tumor cells, immune cells, other cells of the tumor microenvironment, etc.). The term “positive tumor cell fraction”, which may be abbreviated “% TC” is the percentage of viable tumor cells showing positive staining for a protein of interest in one or more cellular location. A “tumor cell”, as used herein, refers to a cancerous cell. Accordingly, a positive tumor cell fraction may be calculated using the formula

$$\% TC = \frac{number of positive viable tumor cells}{number of positive + negative viable tumor cells} \times 100 ,$$

wherein all positive-staining non-tumor cells (e.g., tumor-infiltrating immune cells, normal cells, necrotic cells, and debris) are excluded from evaluation and scoring. The term “positive immune cell fraction”, which may be abbreviated “% IC” is the percentage of viable immune cells in a sample or specified area of a sample showing positive staining for a protein of interest in one or more cellular location.

An “H-Score” is another means for characterizing expression level of a protein and captures both the intensity and the proportion of the biomarker of interest from an image (e.g., IHC). Individual viable cells (e.g., tumor cells, immune cells, other cells of the tumor microenvironment, and combinations thereof), and in some embodiments their sub-cellular compartments (e.g., nucleus, cytoplasm, cell membrane, etc.), are first detected and based on the relative expression of the biomarker of interest in the cell, or one or more sub-cellular compartments, the cells are classified as either positive or negative. The positive cells are further classified into high (3+), medium (2+), or low (1+) based on the biomarker signal intensity. The H-score (range of 0 to 300) is calculated using the following equation: (3×% cells staining at 3+)+(2×% cells staining at 2+)+(1×% cells staining at 1+). A tumor H-score refers to an H-score calculated based on the relative expression of a biomarker of interest in viable tumor cells.

Another means for characterizing expression level of a protein that captures both the intensity and proportion of the biomarker of interest from an image (e.g., IHC) is “2+ or 3+ % TC”, which is the percentage of viable tumor cells showing positive staining for a protein of interest in one or more cellular location (e.g., cell membrane, cytoplasm) with a signal intensity of either medium (2+) or high (3+). Accordingly, 2+ or 3+ % TC may be calculated using the formula

$$2 + or 3 + \% TC = \frac{\begin{matrix}number of positive viable tumor \\ cells with medium or high intensity\end{matrix}}{number of positive + negative viable tumor cells} \times 100 ,$$

wherein all positive-staining non-tumor cells (e.g., tumor-infiltrating immune cells, normal cells, necrotic cells, and debris) are excluded from evaluation and scoring.

The term “protein,” as used herein, encompasses “full-length,” unprocessed protein as well as any form of the protein that results from processing in the subject (e.g., processing that may occur intracellularly or extracellularly within the body). The term also encompasses naturally occurring variants of the protein, e.g., splice variants or allelic variants.

“Polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The terms “polynucleotide” and “nucleic acid” specifically includes mRNA and cDNAs.

The terms “patient” or “subject” are used interchangeably to refer to a human.

The terms “treat”, “treating”, treatment” and the like refer to a course of action that eliminates, reduces, suppresses, mitigates, ameliorates, or prevents the worsening of, either temporarily or permanently, a disease, disorder or condition to which the term applies, or at least one of the symptoms associated therewith. Treatment includes alleviation of symptoms, diminishment of extent of disease, inhibiting (e.g., arresting the development or further development of the disease, disorder or condition or clinical symptoms association therewith) an prolonging survival of a subject as compared to expected survival if not receiving treatment or as compared to a published standard of care therapy for a particular disease.

The term “in need of treatment” as used herein refers to a judgment made by a physician or similar professional that a subject requires or will benefit from treatment. This judgment is made based on a variety of factors that are in the realm of the physician's expertise, which may include a positive diagnosis of a disease, disorder or condition.

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a KD for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. The term “specifically binds” may also refer to binding where a molecule (e.g., an antibody or antigen binding fragment thereof) binds to a particular polypeptide, or an epitope on a particular polypeptide, without substantially binding to any other polypeptide, or polypeptide epitope.

The term “antibody,” as used herein, is used in the broadest sense and encompasses various antibody and antibody-like structures that specifically bind to a single antigen or to multiple antigens (e.g., monospecific antibodies, multispecific antibodies, polyepitopic antibodies, etc.), including but not limited to full-length antibodies, antigen-binding fragments, heavy chain antibodies, single-chain antibodies, and higher order variants of single-chain antibodies. Thus, any reference to an antibody should be understood to refer to the antibody in intact form or an antigen-binding fragment (including an antigen-binding fragment derived from a full-length antibody) unless the context requires otherwise. Preferably, but not necessarily, antibodies useful herein are isolated and can be produced recombinantly. The term “isolated antibody” refers to an antibody that has been separated from a component of its natural environment. In some embodiments, an isolated antibody is purified to greater than 95% or 99% purity as determined by, for example, electrophoresis or chromatography (e.g., ion exchange or reverse phase HPLC).

The terms “full-length antibody,” “intact antibody,” and “whole antibody” are used herein interchangeably to refer to an antibody having a structure substantially similar to a native antibody structure or having heavy chains that contain an Fc region.

The term “antibody fragment” refers to a molecule other than an intact antibody that comprises a portion of an intact antibody that binds the antigen to which the intact antibody binds. Examples of antigen-binding fragment include, without limitation, a diabody, a Fab, a Fab′, a F(ab′)2, a F(ab)c, an Fv fragment, a disulfide stabilized Fv fragment (dsFv), a (dsFv)2, a bispecific dsFv (dsFv-dsFv′), a disulfide stabilized diabody (ds diabody), a triabody, a tetrabody, a single-chain antibody, an scFv, an scFv dimer, a single domain antibody, a single-domain antibody, and a multivalent domain antibody. Typically, binding fragments compete with the intact antibody from which they were derived for specific binding. Binding fragments can be produced by recombinant DNA techniques, or by enzymatic or chemical separation of intact immunoglobulins.

The term “Fc region” is used herein to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the Eu numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

A “functional Fc region” possesses an effector function of a native sequence Fc region. Exemplary effector functions include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g., an antibody variable domain) and can be assessed using various assays disclosed herein or otherwise known in the art. A functional Fc region may possess effector function substantially similar to a wild-type IgG, reduced (but still measurable) effector function compared to a wild-type IgG, or enhanced effector function compared to a wild-type IgG.

A “native sequence Fc region” comprises an amino acid sequence identical to the amino acid sequence of an Fe region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof. A “variant Fc region” comprises an amino acid sequence which differs from that of a native sequence Fc region by virtue of at least one amino acid modification (e.g., from about one to about ten amino acid modifications, and in some embodiments from about one to about five amino acid modifications), preferably one or more amino acid substitution(s). The variant Fc region herein will preferably possess at least about 80% homology with a native sequence Fc region and/or with an Fc region of a parent polypeptide, preferably at least about 90% homology therewith, or preferably at least about 95% homology therewith. In some embodiments, variant Fc regions may possess reduced effector function (including no effector function) compared to a wild-type IgG. In other embodiments, variant Fc regions may possess enhanced effector function, as compared to a wild-type IgG.

As used herein, the term “therapeutic agent” is intended to mean a compound that, when present in an effective amount, produces a desired therapeutic effect on a subject in need thereof.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a value in the range of about 1 to about 200 should be understood to include the explicitly recited limits of about 1 and about 200, but also to include individual values such as about 2, about 3, and about 4, and sub-ranges such as about 10 to about 50, about 20 to about 100, and so forth.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “optional” or “optionally” means that the subsequently described event or circumstance can or cannot occur, and that the description includes instances where the event or circumstance occurs and instances where it does not.

### Biomarkers of the Disclosure

The present disclosure relates to a biomarker or a combination of biomarkers (e.g., 2, 3, 4, 5 or more), and use of these biomarkers individually or in combination in clinical and diagnostic settings to affect treatment. In various embodiments, a biomarker of the present disclosure may be an expression level of a protein (e.g., a cytokine, or a protein expressed by an immune cell and/or a protein expressed by a tumor cell and/or or a protein expressed by another cell type in the tumor microenvironment such as a stromal cell, a fibroblast, an endothelial cell, etc.), or a nucleic acid encoding said protein, relative to a reference level. For clarity, the term “expression level” when used in regard to a protein (e.g., a PD-L1 expression level, a CD155 expression level, etc.), may refer to a detectable level of the polynucleotide (mRNA) encoding the protein and in some instances fragments thereof or to a detectable level of the translated polypeptide and in some instances fragments thereof. The polynucleotide and/or protein may be tumor-associated, e.g., detectable in or on tumor cells or other cells within the tumor microenvironment (e.g., immune cells, stromal cells, fibroblasts, endothelial cells, etc.), or may be circulating, e.g., detectable in or on intact cells or subcellular structures (e.g., exosomes) in blood or lymph or extracts thereof, or detectable as a soluble form in blood, lymph or extracts thereof. In other embodiments, a biomarker of the present disclosure may be the presence, absence, or amount of a genomic feature of a tumor (e.g., mutation, deletion, amplification, rearrangement, change in copy number, etc.) relative to a reference level. Expression levels or amounts equal to or above the reference level are “high” and expression levels or amounts below the reference level are “low”. Accordingly, a biomarker high tumor (e.g., a CD155 high tumor, etc.) is a tumor where the expression level of the biomarker is equal to or above the reference level in a sample of the tumor. Similarly, high expression of a peripheral biomarker (e.g., high CXCL10 expression in a blood derived sample, etc.) refers to an expression level of the biomarker, measured in the peripheral sample, that is equal to or above a reference level. Non-limiting examples of uses contemplated herein for the biomarkers of the present disclosure include identifying subjects with cancer that have a poor prognosis, improving outcomes for subjects with a poor prognosis, determining effectiveness of a therapy (e.g., immune checkpoint inhibitor) for treating cancer in subjects, identifying subjects for treatment with an immune checkpoint inhibitor or a combination of immune checkpoint inhibitors, identifying subjects for a clinical trial and/or stratifying subjects into treatment arms during a clinical trial (e.g., based on prognosis and/or predicted responsiveness to immune checkpoint inhibitors), determining whether a cancer is likely to respond to a therapy (e.g., an immune checkpoint inhibitor), and the like. As described in further detail herein, the effectiveness of a therapy comprising an immune checkpoint inhibitor (e.g., PD-(L)1 antagonist, TIGIT antagonist) or a combination of immune checkpoint inhibitors (PD-(L)1 antagonist and a TIGIT antagonist), for use as a medicament for treating a cancer, may be improved by administering the therapy to a subject with a cancer characterized by one or more biomarker disclosed herein. Improved effectiveness may be demonstrated in a clinical trial setting, for example by comparing an outcome measured in an experimentally treated cohort of subjects to the same outcome measured in a biomarker-selected subset of that cohort (e.g., prospectively or retrospectively selected based on the biomarker or combination of biomarkers). Non-limiting examples of suitable outcomes include response rates (e.g., measures of complete responses, partial responses, stable disease, progressive disease, objective response rate, etc.), duration of response, time to response, progression free survival, and overall survival. Improved effectiveness does not require a therapy to always be effective in treating every individual subject of the biomarker selected cohort.

When a biomarker is a protein expression level (e.g., an expression level of a cytokine, PD-L1, PD-1, TIGIT, CD226, CD155 or a protein of the ATP-adenosine axis (e.g., CD39, CD73, TNAP, A₂ₐR, A_{2b}R), expression level may be measured in a sample by immunohistochemistry (IHC), immunocytochemistry, immunofluorescence microscopy, immunophenotyping by flow cytometry, ELISA, electrochemiluminescence-based immunoassays or other immunoassays, mass spectrometry, proximity extension assays (e.g., Olink assays), aptamer-based assays (e.g., SOMAscan assays), etc. Non-limiting examples of suitable samples include tissue, cells and/or fluid from biopsies (e.g., bone marrow biopsies, lymph node biopsies, skin biopsies, surgical biopsies, tumor biopsies, etc.) or resections, tumor lysates, tissue extracts such as homogenized tissue, lymph, blood, blood-derived cells, plasma, and serum. Protein expression levels may be characterized in a number of different ways using different algorithms. For example, protein expression level measured by IHC may be characterized by percent cell positivity, percent cell positivity relative to an area, an H-score, percent positivity at a given intensity level, and the like. Protein expression level measured, for example, by ELISA, immunoassay, mass spectrometry, proximity extension assays, aptamer-based assays, and the like may be characterized by an absolute amount, a relative amount, or a fold-change. For methods that use an antibody to detect the protein of interest (e.g., IHC), it will be appreciated that any given antibody that specifically binds the protein may be used with a particular assay protocol and/or scoring terminology to derive protein expression level. Sensitivities can vary between different antibodies and therefore thresholds defined by one particular assay may not be applicable to another, even if using the same scoring algorithm. For example, only about 64% of samples that meet a PD-L1 expression level threshold of 1% TC or 25% TC, as defined respectively by staining with anti-PD-L1 antibodies 28-8 or 22C3 and SP263 in an IHC assay, meet the threshold when stained using anti-PD-L1 antibody SP142 (Hirsch et al., Journal of Thoracic Oncology 2016, 12(2): 208-222). Although there may not be agreement between absolute threshold values, different assays and algorithms—when used at different thresholds—may identify the same population of patients and concordance between different approaches may be empirically determined by a person of skill in the art (Liu et al., “Tumor Area Positivity (TAP) score of programmed death-ligand 1 (PD-L1): a novel visual estimation method for combined tumor cell and immune cell scoring,” doi.org/10.21203/rs.3.rs-2206120/v1).

When a biomarker is a polynucleotide expression level greater than or equal to a reference level, or lower than a reference level, expression level may be measured by transcriptomic profiling of tumor or immune cells or stromal cells and other cells present in the tumor microenvironment in a sample, for example by next-generation sequencing (NGS), RNA sequencing (RNA-Seq), microarrays, quantitative RT-PRC (qRT-PCR), etc. Non-limiting examples of suitable samples include tissue, cells and/or fluids from biopsies (e.g., bone marrow biopsies, lymph node biopsies, skin biopsies, surgical biopsies, tumor biopsies, etc.) or resections, tumor lysates, tissue extracts such as homogenized tissue, lymph, blood, blood-derived cells and plasma.

When a biomarker is presence, absence, or amount of a genomic feature of a tumor greater than or equal to a reference level, or lower than a reference level, the genomic feature may be detected and/or measured by next-generation sequencing (NGS), fluorescence in situ hybridization (FISH), or whole exome sequencing (WES). Non-limiting examples of suitable samples include tissue, cells and/or fluids from biopsies (e.g., bone marrow biopsies, lymph node biopsies, skin biopsies, surgical biopsies, tumor biopsies, etc.) or resections, tumor lysates, tissue extracts such as homogenized tissue, lymph, blood, blood-derived cells and plasma.

In some embodiments, a biomarker of the present disclosure is PD-L1 expression level greater than or equal to a reference level or lower than a reference level. The term “PD-L1” or “Programmed Cell Death Ligand 1” refers herein to a protein in humans encoded by the CD274 gene and naturally occurring variants of PD-L1, e.g., splice variants, or allelic variants, or naturally occurring fragments of PD-L1, e.g., originating from a transcript generated by alternative splicing or a degraded transcript, or from a post-translational processing of the polypeptide, e.g., by proteolysis. The amino acid sequence of an exemplary human PD-L1 may be found under UniProt Accession Number Q9NZQ7. When PD-L1 expression is detected by immunohistochemistry (IHC), for example by an IHC assay staining for PD-L1 using an antibody that specifically binds to PD-L1 (e.g., SP263, 22C3, SP142, 28-8, etc.), PD-L1 positive staining may refer to partial or complete membrane staining (exclusive of cytoplasmic staining) at any intensity or may refer to membranous, cytoplasmic, and punctate staining at any intensity, depending upon the particular IHC assay and tumor type being evaluated.

In some embodiments, PD-L1 expression level is measured using an immunohistochemistry (IHC) assay for PD-L1. For example, PD-L1 expression level may be measured in a sample stained with SP263, 22C3, SP142, or 28-8 using OPTIVIEW® detection on Benchmark ULTRA, EnVision Flex on AutostainerLink 48, OPTIVIEW® detection and amplification on Benchmark ULTRA, or EnVision Flex on AutostainerLink 48, respectively. In some embodiments, PD-L1 expression level is measured using a companion diagnostic test regulated by a health authority (e.g., U.S. Food & Drug Administration, European Medicines Agency, etc.), optionally selected from the Ventana SP263 IHC assay, the Ventana SP142 IHC assay, the pharmDx 28-8 IHC assay, or the pharmDx 22C3 IHC assay. As used herein, the “Ventana SP263 IHC assay” is a PD-L1 IHC assay conducted according to the Ventana PD-L1 (SP263) Assay package insert (Tucson, Ariz.: Ventana Medical Systems, Inc.), which is incorporated herein by reference in its entirety. As used herein, the “Ventana SP142 IHC assay” is a PD-L1 IHC assay conducted according to the Ventana PD-L1 (SP142) Assay package insert (Tucson, Ariz.: Ventana Medical Systems, Inc.), which is incorporated herein by reference in its entirety. As used herein, the “pharmDx 28-8 IHC assay” is a PD-L1 IHC assay conducted according to the PH-L1 IHC 28-8 pharmDx package insert (Carpinteria, Calif.: Dako, Agilent Pathology Solutions), which is incorporated herein by reference in its entirety. As used herein, the “pharmDx 22C3 IHC assay” is a PD-L1 IHC assay conducted according to the PD-L1 IHC 22C3 pharmDx package insert (Carpinteria, Calif.: Dako, Agilent Pathology Solutions), which is incorporated herein by reference in its entirety.

In some embodiments, PD-L1 expression level for a sample is characterized by a PD-L1-positive tumor cell fraction. As used herein, “PD-L1-positive tumor cell fraction” or “PD-L1 % TC” or “tumor proportion score (TPS)” are used interchangeably, and is the percentage of viable tumor cells showing partial or complete membrane staining at any intensity (≥1+) that is distinct from cytoplasmic staining, e.g., following staining of the sample using an immunohistochemical (IHC) assay for PD-L1, e.g., an IHC assay using an anti-PD-L1 antibody, such as SP263, 22C3, SP142, or 28-8. Accordingly, a PD-L1-positive tumor cell fraction may be calculated using the formula

$$PD - L 1 \% TC = \frac{number of PD - L 1 - positive tumor cells}{number of PD - L 1 - positive + PD - L 1 negative tumor cells} \times 100 ,$$

wherein PD-L1 cytoplasmic staining of tumor cells and all non-tumor cells (e.g., tumor-infiltrating immune cells, normal cells, necrotic cells, and debris) are excluded from evaluation and scoring. In some embodiments, the reference level may be 1% TC, 10% TC, 50% TC, or 75% TC. Samples that are ≤1% TC may be referred to as having “no PD-L1 expression”; samples that are ≥1% TC may be referred to as being “PD-L1 positive”; and samples that are ≥50% TC may be referred to as being “PD-L1 high”.

In some embodiments, a biomarker of the present disclosure is PD-L1 expression level that is <1% TC. In some embodiments, a biomarker of the present disclosure is PD-L1 expression level that is ≥1% TC. In some embodiments, a biomarker of the present disclosure is PD-L1 expression level that is ≥50% TC or ≥75% TC. In some embodiments, a biomarker of the present disclosure is PD-L1 expression level that is <50% TC. In some embodiments, a biomarker of the present disclosure is PD-L1 expression level that is 1-49% TC. In some embodiments, a biomarker of the present disclosure is PD-L1 expression level that is ≥10% TC. In some embodiments, a biomarker of the present disclosure is PD-L1 expression level that is >10% TC. In some embodiments, a biomarker of the present disclosure is PD-L1 expression level that is ≥50% TC. In some embodiments, the sample is/was obtained from a subject with cancer (e.g., a solid tumor, e.g., lung cancer, e.g., NSCLC, e.g., squamous or non-squamous NSCLC, e.g., locally advanced unresectable NSCLC (e.g., Stage III NSCLC), or recurrent or metastatic NSCLC (e.g., Stage IV NSCLC)), and PD-L1 expression level for the sample is characterized by a PD-L1-positive tumor cell fraction, optionally using the pharmDx 22C3 IHC assay, the pharmDx 28-8 IHC assay, or the Ventana SP263 IHC assay.

In some embodiments, PD-L1 expression level for a sample is characterized by PD-L1 positive immune cell staining, or by PD-L1 positive tumor cell staining and PD-L1 positive immune cell staining. In some embodiments PD-L1 expression level for a sample is characterized by a Combined Positive Score (CPS). In some embodiments, PD-L1 expression level for a sample is characterized by Tumor Area Proportion (TAP). In some embodiments PD-L1 expression level for a sample is characterized by a PD-L1-positive immune cell fraction (% IC).

“CPS” refers to the number of PD-L1 positive tumor cells and PD-L1 positive mononuclear immune cells (MIC, e.g., lymphocytes, macrophages) within tumor nests and adjacent supporting stroma showing partial or complete membrane staining at any intensity that is distinct from cytoplasmic staining (i.e., staining at a score of ≥1+) relative to all viable tumor cells present in a sample, e.g., following staining of the sample using an IHC assay for PD-L1, e.g., an IHC assay using the antibody SP263, 22C3, SP142, or 28-8. Accordingly, a PD-L1-positive tumor cell fraction may be calculated by the formula

$$CPS = \frac{PD - L 1 positive tumor cells + PD - L 1 positive MIC}{PD - L 1 positive tumor cells + PD - L 1 negative tumor cells} \times 100 ,$$

wherein PD-L1 cytoplasmic staining of tumor cells and non-tumor cells (e.g., tumor-infiltrating immune cells, normal cells, necrotic cells, and debris) are excluded from evaluation and scoring. In various embodiments, the reference level may be CPS 1, 2, 3, 4, 5, 6, 7, 8, 9, 10, 15, 20, 25, 30, 35, 40, 45, or 50.

In some embodiments, a biomarker of the present disclosure is PD-L1 expression level that is CPS <1. In some embodiments, a biomarker of the present disclosure is PD-L1 expression level that is CPS ≥1. In some embodiments, a biomarker of the present disclosure is PD-L1 expression level that is CPS <5. In some embodiments, a biomarker of the present disclosure is PD-L1 expression level that is CPS ≥5. In some embodiments, a biomarker of the present disclosure is PD-L1 expression level that is CPS <10. In some embodiments, a biomarker of the present disclosure is PD-L1 expression level that is CPS ≥10. In some embodiments, the sample is/was obtained from a subject with cancer (e.g., a solid tumor, e.g., a cancer other than lung cancer, e.g., an upper GI cancer, e.g., gastric, gastroesophageal junction (GEJ) or esophageal adenocarcinoma EAC, e.g., locally advanced unresectable gastric, GEJ or EAC (e.g., Stage III gastric, GEJ or EAC), or recurrent or metastatic gastric, GEJ or EAC (e.g., Stage IV gastric, GEJ or EAC)), and PD-L1 expression level for the sample is characterized by CPS, optionally using the pharmDx 22C3 IHC assay, the pharmDx 28-8 IHC assay, or the Ventana SP263 IHC assay.

“TAP” refers to the percent area of PD-L1 positive tumor cells showing partial or complete membrane staining at any intensity that is distinct from cytoplasmic staining and the percent area of PD-L1 positive tumor-associated immune cells showing membranous, cytoplasmic, and punctate staining at any intensity relative to tumor area. Accordingly, a PD-L1-positive tumor and immune cell fraction may be calculated by the formula TAP=(% PD-L1 positive TC and IC)/Tumor area

$$TAP = \frac{\begin{matrix}PD - L 1 positive tumor cells + \\ PD - L 1 positive tumor - associated immune cells\end{matrix}}{tumor area} \times 100.$$

When quantifying PD-L1 expression by TAP, tumor-associated immune cells are intra- and peri-tumoral, including those present within the tumor proper, between tumor nests, and within any tumor-associated reactive stroma. In lymph nodes with focal or discrete tumor metastases, only immune cells immediately adjacent to the leading edge of the metastatic tumor nest are typically defined as tumor-associated immune cells. In various embodiments, the reference level may be TAP 1%, 2%, 3%, 4%, 5%, 6%, 7%, 8%, 9%, 10%, 15%, 20%, 25%, 30%, 35%, 40%, 45%, or 50%.

In some embodiments, a biomarker of the present disclosure is PD-L1 expression level that is TAP <1%. In some embodiments, a biomarker of the present disclosure is PD-L1 expression level that is TAP ≥1%. In some embodiments, a biomarker of the present disclosure is PD-L1 expression level that is TAP <5%. In some embodiments, a biomarker of the present disclosure is PD-L1 expression level that is TAP ≥5%. In some embodiments, a biomarker of the present disclosure is PD-L1 expression level that is TAP <10%. In some embodiments, a biomarker of the present disclosure is PD-L1 expression level that is TAP ≥10%. In some embodiments, the sample is/was obtained from a subject with cancer (e.g., a solid tumor, e.g., a cancer other than lung cancer, e.g., an upper GI cancer, e.g., gastric, gastroesophageal junction (GEJ) or esophageal adenocarcinoma EAC, e.g., locally advanced unresectable gastric, GEJ or EAC (e.g., Stage III gastric, GEJ or EAC), or recurrent or metastatic gastric, GEJ or EAC (e.g., Stage IV gastric, GEJ or EAC)), and PD-L1 expression level for the sample is characterized by TAP, optionally using the pharmDx 22C3 IHC assay, the pharmDx 28-8 IHC assay, or the Ventana SP263 IHC assay.

“PD-L1-positive immune cell fraction”, abbreviated “PD-L1 % IC”, is the percentage of viable tumor-infiltrating immune cells showing membranous or cytoplasmic staining at any intensity (21+) as a proportion of tumor area including associated intratumoral and contiguous peritumoral stroma, e.g., following staining of the sample using an immunohistochemical (IHC) assay for PD-L1, e.g., an IHC assay using an anti-PD-L1 antibody, such as SP263, 22C3, SP142, or 28-8. In some embodiments, the reference level may be 1% IC, 5% IC, 10% IC, 50% IC, or 75% IC.

In some embodiments, a biomarker of the present disclosure is PD-L1 expression level that is <1% IC. In some embodiments, a biomarker of the present disclosure is PD-L1 expression level that is ≥1% IC. In some embodiments, a biomarker of the present disclosure is PD-L1 expression level that is <5% IC. In some embodiments, a biomarker of the present disclosure is PD-L1 expression level that is ≥5% IC. In some embodiments, a biomarker of the present disclosure is PD-L1 expression level that is <10% IC. In some embodiments, a biomarker of the present disclosure is PD-L1 expression level that is ≥10% IC.

In some embodiments, a biomarker of the present disclosure is CD155 expression level greater than or equal to a reference level or lower than a reference level. The terms “CD155” and “Poliovirus receptor” and “PVR” refer to a protein that is encoded in humans by the PVR gene and naturally occurring variants of PVR or CD155, e.g., splice variants, or allelic variants, or naturally occurring fragments of CD155, e.g., originating from a transcript generated by alternative splicing or a degraded transcript, or from a post-translational processing of the polypeptide, e.g., by proteolysis. The amino acid sequence of an exemplary human CD155 may be found under UniProt Accession Number P15151. When CD155 expression is detected by immunohistochemistry (IHC), for example by an IHC assay staining for CD155 using an antibody that specifically binds to CD155 (e.g., rabbit clone D3G7H (Cell Signaling), rabbit clone D8A5G (Cell Signaling), etc.), CD155 positive staining may refer to partial or complete membrane staining that is distinct from cytoplasmic staining, at any intensity, or may refer to membranous and cytoplasmic staining at any intensity, depending upon the particular IHC assay, tumor type and cell type being evaluated. In various embodiments, CD155 expression level for a sample may be characterized by tumor cell staining, immune cell staining, staining of other cell types (e.g., normal adjacent tissue (NAT), endothelia, smooth muscle, fibroblast, stroma), or any combination thereof. Exemplary methods for characterizing CD155 expression level are described in further detail in Example 1. The present disclosure describes uses of CD155 expression levels greater than or equal to a reference level, or lower than a reference level, as a prognostic and/or predictive biomarker. In these embodiments, CD155 expression may identify subjects with differentiated immunological or biological attributes. Methods for identifying these differentiated immunological and/or biological attributes are described herein, including Example 2.

In some embodiments, CD155 expression level for a sample is characterized by a CD155-positive tumor cell fraction. As used herein, “CD155-positive tumor cell fraction” is the percentage of viable tumor cells showing positive staining at any intensity in a sample, following staining of the sample, e.g., in an immunohistochemical (IHC) assay for CD155, e.g., an IHC assay using an anti-CD155 antibody, such as D3G7H. Accordingly, a CD155-positive tumor cell fraction may be calculated using the formula

$$CD 155 \% TC = \frac{number of CD 155 - positive tumor cells}{number of CD 155 - positive + CD 155 - negative tumor cells} \times 100 ,$$

wherein all CD155-staining non-tumor cells (e.g., tumor-infiltrating immune cells, normal cells, necrotic cells, and debris) are excluded from evaluation and scoring. In some embodiments, positive tumor cell staining refers to tumor cells showing partial or complete membrane staining at any intensity that is distinct from cytoplasmic staining. In some embodiments, positive tumor cell staining refers to tumor cells showing partial or complete membrane staining at any intensity and/or cytoplasmic staining. A tumor sample may be considered positive with at least 1% of tumor cells demonstrating positive expression. The reference level when CD155 expression level is characterized by CD155% TC may be a median CD155% TC in a group of subjects with the same disease (e.g., cancer) or may be a value within a range above or below the median. In some embodiments, the reference level may be ≤30%, ≤25%, ≤20%, ≤15%, ≤10%, or ≤5% above or below a median level. In various embodiments, the reference level may be 1% TC, 5% TC, 10% TC, 15% TC, 20% TC, 25% TC, 30% TC, 35% TC, 40% TC, 45% TC, 50% TC, 55% TC, 60% TC, 65% TC, 70% TC, 75% TC, 80% TC, 85% TC, or 90% TC.

In some embodiments, CD155 expression level for a sample is characterized by a CD155 2+ or 3+ % TC. As used herein, “CD155 2+ or 3+ % TC” is the percentage of viable tumor cells showing positive staining at medium or high intensity in a sample, following staining of the sample, e.g., in an immunohistochemical (IHC) assay for CD155, e.g., an IHC assay using an anti-CD155 antibody, such as D3G7H. Accordingly, a CD155 2+ or 3+ % TC may be calculated using the formula

$$CD 155 \; 2 + or 3 + \% TC = \frac{number of CD 155 - positive tumor cells at 2 + or 3 + intensity}{number of CD 155 - positive + CD 155 - negative tumor cells} \times 100 ,$$

wherein all CD155-staining non-tumor cells (e.g., tumor-infiltrating immune cells, normal cells, necrotic cells, and debris) are excluded from evaluation and scoring. In some embodiments, positive tumor cell staining refers to tumor cells showing partial or complete membrane staining at medium or high intensity that is distinct from cytoplasmic staining. In some embodiments, positive tumor cell staining refers to tumor cells showing partial or complete membrane staining at medium or high intensity and/or cytoplasmic staining at medium or high intensity. The reference level, when CD155 expression level is characterized by CD155 2+ or 3+ % TC, may be a median CD155 2+ or 3+ % TC in a group of subjects with the same disease (e.g., cancer) or may be a value within a range above or below the median. In some embodiments, the reference level may be ≤30%, ≤25%, ≤20%, ≤15%, ≤10%, or ≤5% above or below a median level. In some embodiments, the reference level may be 2+ or 3+ 30% TC, 2+ or 3+ 35% TC, 2+ or 3+ 40% TC, 2+ or 3+ 45% TC, 2+ or 3+ 50% TC, 2+ or 3+ 55% TC, 2+ or 3+ 60% TC, 2⁺ or 3+ 65% TC. In some embodiments, the reference level may be 2+ or 3+ 30% TC, 2⁺ or 3+ 35% TC, 2+ or 3+ 40% TC, 2+ or 3+ 45% TC, or 2+ or 3+ 50% TC. In some embodiments, the reference level may be 2+ or 3+ 40% TC, 2+ or 3+ 45% TC, 2+ or 3+ 50% TC, 2⁺ or 3+ 55% TC, 2+ or 3+ 60% TC. In some embodiments, the reference level may be 2+ or 3+ 50% TC. In some embodiments, the reference level may be 2+ or 3+ 7% TC, 2+ or 3+ 8% TC, 2+ or 3+ 9% TC, 2+ or 3+ 10% TC, 2+ or 3+ 11% TC, 2+ or 3+ 12% TC, or 2+ or 3+ 13% TC. In some embodiments, the reference level may be 2+ or 3+ 40% TC. In some embodiments, the reference level may be 2+ or 3+ 35% TC. In some embodiments, the reference level may be 2+ or 3+ 45% TC.

In some embodiments, CD155 expression level for a sample is characterized by a tumor H-score. An H-Score (range of 0 to 300) may be calculated based on the summation of the product of percent of cells stained at each intensity using the following equation: (3×% cells staining at 3+)+(2×% cells staining at 2+)+(1×% cells staining at 1+). The reference level when CD155 expression level is characterized by a tumor H-score may be a median tumor H-score in a group of subjects with the same disease (e.g., cancer) or may be a value within a range above or below the median. In some embodiments, the reference level may be ≤30%, ≤25%, ≤20%, ≤15%, ≤10%, or ≤5% above or below the median. In various embodiments, the reference level may be a tumor H-score of 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300. In some embodiments, the reference level may be a tumor H-score of 95, 100, 105, 110, 115, 120, 125, 130, 135, 140, 145, 150, 155, 160, 165, 170, or 175. In some embodiments, the reference level may be a tumor H-score of 110, 115, 120, 125, 130, 135, 140, 145, 150, 155, or 160. In some embodiments, the reference level may be a tumor H-score of 110, 115, 120, 125, 130, 135, 140, 145, or 150. In some embodiments, the reference level may be a tumor H-score of 120, 125, 130, 135, 140, 145, or 150. In some embodiments, the reference level may be a tumor H-score of 75, 80, 85, 90, 95, 100, 105, 110, 115, 120, 125, 130, or 135. In some embodiments, the reference level may be a tumor H-score of 120, 125, 130, 135, 140, 145, or 150. In some embodiments, the reference level may be a tumor H-score of 95, 100, 105, 110, 115, 120, 125, or 130. In some embodiments, the reference level may be a tumor H-score of 120, 125, 130, 135, 140, 145, or 150. In some embodiments, the reference level may be a tumor H-score of 75, 80, 85, 90, 95, 100, 105, 110, 115, 120, 125, 130, or 135. In some embodiments, the reference level may be a tumor H-score of 25, 30, 35, 40, 45, 50 or 55. In some embodiments, the reference level may be a tumor H-score of 95, 100, 105, 110, 115, 120, 125, or 130.

In some embodiments, a biomarker of the present disclosure is CD155 expression level that is characterized by a tumor H-score <170, <160, <150, <140, <130, <120, <110, <100, or <90. In some embodiments, a biomarker of the present disclosure is CD155 expression level that is characterized by a tumor H-score <150, <145, <140, <135, <130, <125, <120. In some embodiments, a biomarker of the present disclosure is CD155 expression level that is <140. In some embodiments, a biomarker of the present disclosure is CD155 expression level that is ≥140. In some embodiments, a biomarker of the present disclosure is CD155 expression level that is <135. In some embodiments, a biomarker of the present disclosure is CD155 expression level that is ≥135. In some embodiments, a biomarker of the present disclosure is CD155 expression level that is <130. In some embodiments, a biomarker of the present disclosure is CD155 expression level that is ≥130. In some embodiments, a biomarker of the present disclosure is CD155 expression level that is <125. In some embodiments, a biomarker of the present disclosure is CD155 expression level that is ≥125.

In some embodiments, CD155 expression level is characterized relative to tumor area. For example, in some embodiments CD155 expression level may be characterized by the number of CD155-positive tumor cells showing positive staining at any intensity and/or the number of CD155-positive tumor-associated immune cells and/or the number of stromal or other cells in the tumor microenvironment showing positive staining at any intensity relative to tumor area. In some embodiments, positive staining refers to cells (tumor or immune) showing partial or complete membrane staining at any intensity that is distinct from cytoplasmic staining. In some embodiments, positive staining refers to cells (tumor or immune) showing partial or complete membrane staining at any intensity and/or cytoplasmic staining. Tumor-associated immune cells may be intra- and peri-tumoral, including those present within the tumor proper, between tumor nests, and within any tumor-associated reactive stroma. In lymph nodes with focal or discrete tumor metastases, immune cells immediately adjacent to the leading edge of the metastatic tumor nest are typically defined as tumor-associated immune cells. The reference level when CD155 expression level is characterized relative to tumor may be a median score in a group of subjects with the same disease (e.g., cancer) or may be a value within a range above or below the median. In some embodiments, the reference level may be ≤30%, ≤25%, ≤20%, ≤15%, ≤10%, or ≤5% above or below the median.

In some embodiments, the CD155 expression level for a sample is characterized by a cyto-membranous 2+ or 3+ % TC, in which the positive staining in the membrane and cytoplasm are combined. In some embodiments, the reference level may be ≤30%, ≤25%, ≤20%, ≤15%, ≤10%, or ≤5% above or below the median (e.g., 10% in gastric-intestinal cancers). In various embodiments, the reference level may be a cyto-membranous 2⁺ or 3+ % TC of 8%, 9%, 10%, 11%, 12%, 13%, 14%, 15%, 16%, 17% or 18%. In various embodiments, the reference level may be a cyto-membranous 2+ or 3+ % TC of 8%, 9%, 10%, 11%, 12%, 13%, 14%, 15%, or 16%. In various embodiments, the reference level may be a cyto-membranous 2+ or 3+ % TC of 8%, 9%, 10%, 11%, 12%, 13%, 14%, or 15%. In various embodiments, the reference level may be a cyto-membranous 2+ or 3+ % TC of 8%, 9%, 10%, 11%, 12%, 13%, or 14%. In various embodiments, the reference level may be a cyto-membranous 2+ or 3+ % TC of 9%, 10%, 11%, 12%, or 13%. In various embodiments, the reference level may be a cyto-membranous 2+ or 3+ % TC of 9%, 10%, 11%, or 12%. In various embodiments, the reference level may be a cyto-membranous 2+ or 3+ % TC of 9%, 10%, or 11%.

In some embodiments, the CD155 expression level for a sample is characterized by a membranous 2+ or 3+ % TC. In some embodiments, the reference level may be ≤30%, ≤25%, ≤20%, ≤15%, ≤10%, or ≤5% above or below the median (e.g., 10% in gastric-intestinal cancers). In various embodiments, the reference level may be a membranous 2⁺ or 3+ % TC of 8%, 9%, 10%, 11%, 12%, 13%, 14%, or 15%. In various embodiments, the reference level may be a membranous 2+ or 3+ % TC of 8%, 9%, 10%, 11%, 12%, 13%, or 14%. In various embodiments, the reference level may be a membranous 2+ or 3+ % TC of 9%, 10%, 11%, 12%, or 13%. In various embodiments, the reference level may be a membranous 2+ or 3+ % TC of 9%, 10%, 11%, or 12%. In various embodiments, the reference level may be a membranous 2+ or 3+ % TC of 9%, 10%, or 11%.

In some embodiments, the CD155 expression level for a sample is characterized by a cyto-membranous H-score, in which the positive staining in the membrane and cytoplasm are combined. In some embodiments, the reference level may be ≤30%, ≤25%, ≤20%, ≤15%, ≤10%, or ≤5% above or below the median (e.g., 105 in gastric-intestinal cancers). In various embodiments, the reference level may be a cyto-membranous H-score of 100, 101, 102, 103, 104, 105, 106, 107, 108, 109, 110, 111, 112, 113, 114 or 115. In various embodiments, the reference level may be a cyto-membranous H-score of 100, 101, 102, 103, 104, 105, 106, 107, 108, 109, 110, 111, 112, or 113. In various embodiments, the reference level may be a cyto-membranous H-score of 101, 102, 103, 104, 105, 106, 107, 108, 109, 110, 111, or 112. In various embodiments, the reference level may be a cyto-membranous H-score of 102, 103, 104, 105, 106, 107, 108, 109, or 110. In various embodiments, the reference level may be a cyto-membranous H-score of 103, 104, 105, 106, 107, or 108. In various embodiments, the reference level may be a cyto-membranous H-score of 104, 105, or 106.

In some embodiments, the CD155 expression level for a sample is characterized by a-membranous H-score. In some embodiments, the reference level may be ≤30%, ≤25%, ≤20%, ≤15%, ≤10%, or ≤5% above or below the median (e.g., 39 in gastric-intestinal cancers). In various embodiments, the reference level may be a membranous H-score of 22, 24, 26, 28, 30, 32, 34, 36, 38, 40, 42, 44, 46, 48 or 50. In various embodiments, the reference level may be a membranous H-score of 24, 26, 28, 30, 32, 34, 36, 38, 39, 40, 42, 44, 46, or 48. In various embodiments, the reference level may be a membranous H-score of 26, 28, 30, 32, 34, 36, 38, 39, 40, 42, 44, or 46. In various embodiments, the reference level may be a membranous H-score of 28, 30, 32, 34, 36, 38, 39, 40, 42, or 44. In various embodiments, the reference level may be a membranous H-score of 32, 34, 36, 38, 39, 40, 42, or 44. In various embodiments, the reference level may be a membranous H-score of 34, 36, 38, 39, 40, or 42. In various embodiments, the reference level may be a membranous H-score of, 38, 39, or 40.

In some embodiments, a biomarker of the present disclosure is CD226 expression level greater than or equal to a reference level or lower than a reference level. The terms “CD226” or “DNAX Accessory Molecule-1” and “DNAM-1” refer to a protein that is encoded in humans by the CD226 gene and naturally occurring variants of CD226, e.g., splice variants, or allelic variants, or naturally occurring fragments of CD226, e.g., originating from a transcript generated by alternative splicing or a degraded transcript, or from a post-translational processing of the polypeptide, e.g., by proteolysis. The amino acid sequence of an exemplary human CD226 may be found under UniProt Accession Number Q15762. When CD226 expression is detected by immunohistochemistry (IHC), for example by an IHC assay staining for CD226 using an antibody that specifically binds to CD226 (e.g., rabbit clone 102 (Sino Biological), rabbit clone E8L9G (Cell Signaling), etc.), CD226 positive staining may refer to partial or complete membrane staining that is distinct from cytoplasmic staining, at any intensity, or may refer to membranous and cytoplasmic staining at any intensity, depending upon the particular IHC assay, tumor type and cell type being evaluated. In various embodiments, CD226 expression level for a sample may be characterized by immune cell staining. Exemplary methods for characterizing CD226 expression level are described in further detail in Example 1.

In some embodiments, CD226 expression level for a sample is characterized by a CD226-positive immune cell fraction. As used herein, “CD226-positive immune cell fraction” is the percentage of viable immune cells showing positive staining at any intensity in a sample, following staining of the sample, e.g., in an immunohistochemical (IHC) assay for CD226, e.g., an IHC assay using an anti-CD226 antibody, such as 102. Accordingly, a CD226-positive immune cell fraction may be calculated using the formula

$$DNAM \% IC = \frac{number of CD 226 - positive immune cells}{number of CD 226 - positive + CD 226 - negative immune cells} \times 100 ,$$

wherein all CD226-staining non-immune cells are excluded from evaluation and scoring. In some embodiments, positive immune cell staining refers to immune cells showing partial or complete membrane staining at any intensity that is distinct from cytoplasmic staining. In some embodiments, positive immune cell staining refers to immune cells showing partial or complete membrane staining at any intensity and/or cytoplasmic staining. An immune sample may be considered positive with at least 1% of immune cells demonstrating positive expression. The reference level when CD226 expression level is characterized by CD226% TC may be a median CD226% IC in a group of subjects with the same disease (e.g., cancer) or may be a value within a range above or below the median. In some embodiments, the reference level may be ≤30%, ≤25%, ≤20%, ≤15%, ≤10%, or ≤5% above or below a median level. In various embodiments, the reference level may be 1% IC, 5% IC, 10% IC, 15% IC, 20% IC, 25% IC, 30% IC, 35% IC, 40% IC, 45% IC, 50% IC, 55% IC, 60% IC, 65% IC, 70% IC, 75% IC, 80% IC, 85% IC, or 90% IC.

In some embodiments, a biomarker of the present disclosure is CD226 expression level that is characterized by ≥1% IC, ≥5% IC, ≥10% IC, ≥15% IC, or ≥20% IC. In some embodiments, a biomarker of the present disclosure is CD226 expression level that is characterized by ≥1% IC, ≥2% IC, ≥3% IC, ≥4% IC, ≥5% IC, ≥6% IC, ≥7% IC, ≥8% IC, ≥9% IC, or ≥10% IC. In some embodiments, a biomarker of the present disclosure is CD226 expression level that is <5. In some embodiments, a biomarker of the present disclosure is CD226 expression level that is ≥5.

In some embodiments, CD226 expression level is characterized relative to tumor area. For example, in some embodiments CD226 expression level may be characterized by the number of CD226-positive tumor-associated immune cells and/or stromal cells and other cells of the tumor microenvironment showing positive staining at any intensity relative to tumor area. In some embodiments, positive staining refers to immune cells showing partial or complete membrane staining at any intensity that is distinct from cytoplasmic staining. In some embodiments, positive staining refers to immune cells showing partial or complete membrane staining at any intensity and/or cytoplasmic staining. Tumor-associated immune cells may be intra- and peri-tumoral, including those present within the tumor proper, between tumor nests, and within any tumor-associated reactive stroma. In lymph nodes with focal or discrete tumor metastases, immune cells immediately adjacent to the leading edge of the metastatic tumor nest are typically defined as tumor-associated immune cells. The reference level when CD226 expression level is characterized relative to tumor may be a median score in a group of subjects with the same disease (e.g., cancer) or may be a value within a range above or below the median. In some embodiments, the reference level may be ≤30%, ≤25%, ≤20%, ≤15%, ≤10%, or ≤5% above or below the median.

In some embodiments, a biomarker of the present disclosure is an adenosine pathway biomarker. As used herein, the term “adenosine pathway biomarker” refers to an indicator of adenosine activity. In some embodiments, an adenosine pathway biomarker is an expression level of a protein greater than or equal to a reference level or lower than a reference level, wherein the protein is CD39, CD73, TNAP, or an adenosine receptor (e.g., A₂ₐR, A_{2b}R), optionally wherein protein expression level is measured by an immunohistochemistry assay. In some embodiments, an adenosine pathway biomarker is polynucleotide expression level of a polynucleotide greater than or equal to a reference level or lower than a reference level, wherein the polynucleotide is a transcribed polynucleotide (i.e., transcript) encoding CD39, CD79, TNAP, or an adenosine receptor (e.g., A₂ₐR, A_{2b}R).

In some embodiments, a biomarker of the present disclosure is CD73 expression level greater than or equal to a reference level or lower than a reference level. The term “CD73” refers herein to a protein in humans encoded by the NT5E gene and naturally occurring variants of CD73, e.g., splice variants or allelic variants, or naturally occurring fragments of CD73, e.g., originating from a transcript generated by alternative splicing or a degraded transcript, or from a post-translational processing of the polypeptide, e.g., by proteolysis. The amino acid sequence of an exemplary human CD73 may be found under UniProt Accession Number P21589. When CD73 expression is detected by immunohistochemistry (IHC), for example by an IHC assay staining for CD73 using an antibody that specifically binds to CD73 (e.g., rabbit clone D7F9A (Cell Signaling), etc.), CD73 positive staining may refer to partial or complete membrane staining that is distinct from cytoplasmic staining, at any intensity, or may refer to membranous and cytoplasmic staining at any intensity, depending upon the particular IHC assay, tumor type and cell type being evaluated. In various embodiments, CD73 expression level for a sample may be characterized by tumor cell staining, immune cell staining, staining of other cell types (e.g., normal adjacent tissue (NAT), endothelia, smooth muscle, fibroblast, stroma), or any combination thereof. Exemplary methods for characterizing CD73 expression level are described in further detail in Example 1.

Use of CD73 expression level as a biomarker may or may not be to indicate or to identify tumors with immunosuppressive adenosine signaling. If CD73 is acting as an indicator of immunosuppressive adenosine signaling (e.g., CD73 “high” tumors), then CD73 expression level can be referred to as “an adenosine pathway biomarker.” The present disclosure also describes uses of CD73 expression levels lower than a reference level as a prognostic and predictive biomarker. In these embodiments, “low” CD73 expression may identify subjects with differentiated immunological or biological attributes. Methods for identifying these differentiated immunological and/or biological attributes are described herein, including Example 2.

In some embodiments, CD73 expression level for a sample is characterized by a CD73-positive tumor cell fraction. As used herein, “CD73-positive tumor cell fraction” is the percentage of viable tumor cells showing positive staining at any intensity in a sample, following staining of the sample, e.g., in an immunohistochemical (IHC) assay for CD73, e.g., an IHC assay using an anti-CD73 antibody, such as D7F9A. Accordingly, a CD73-positive tumor cell fraction may be calculated using the formula

$$CD 73 \% TC = \frac{number of CD 73 - positive tumor cells}{number of CD 73 - positive + CD 73 - negative tumor cells} \times 100 ,$$

wherein all CD73-staining non-tumor cells (e.g., tumor-infiltrating immune cells, normal cells, necrotic cells, and debris) are excluded from evaluation and scoring. In some embodiments, positive tumor cell staining refers to tumor cells showing partial or complete membrane staining at any intensity that is distinct from cytoplasmic staining. In some embodiments, positive tumor cell staining refers to tumor cells showing partial or complete membrane staining at any intensity and/or cytoplasmic staining. A tumor sample may be considered positive with at least 1% of tumor cells demonstrating positive expression. The reference level when CD73 expression level is characterized by CD73% TC may be a median CD73% TC in a group of subjects with the same disease (e.g., cancer) or may be a value within a range above or below the median. As demonstrated herein, a median CD73% TC in a group of subjects with the same disease may be used to identify prognostic and predictive reference levels. In some embodiments, the reference level may be ≤30%, ≤25%, ≤20%, ≤15%, ≤10%, or ≤5% above or below the median. In various embodiments, the reference level may be 1% TC, 5% TC, 10% TC, 15% TC, 20% TC, 25% TC, 30% TC, 35% TC, 40% TC, 45% TC, 50% TC, 55% TC, 60% TC, 65% TC, 70% TC, 75% TC, 80% TC, 85% TC, or 90% TC. In one embodiment, the reference level may be 70% TC, 65% TC, 60% TC, 55% TC, or 50% TC.

In some embodiments, a biomarker of the present disclosure is CD73 expression level that is <80% TC, <75% TC, <70% TC, <65% TC, <60% TC, <55% TC, <50% TC, <55% TC, or <40% TC. In some embodiments, a biomarker of the present disclosure is CD73 expression level that is <65% TC, <60% TC, <55% TC, <50% TC, or <45% TC. In some embodiments, a biomarker of the present disclosure is CD73 expression level that is <65% TC. In some embodiments, a biomarker of the present disclosure is CD73 expression level that is ≥65% TC. In some embodiments, a biomarker of the present disclosure is CD73 expression level that is <60% TC. In some embodiments, a biomarker of the present disclosure is CD73 expression level that is ≥60% TC. In some embodiments, a biomarker of the present disclosure is CD73 expression level that is <55% TC. In some embodiments, a biomarker of the present disclosure is CD73 expression level that is ≥55% TC.

In some embodiments, CD73 expression level for a sample is characterized by a tumor H-score. An H-Score (range of 0 to 300) may be calculated based on the summation of the product of percent of cells stained at each intensity using the following equation: (3×% cells staining at 3+)+(2×% cells staining at 2+)+(1×% cells staining at 1+). The reference level when CD73 expression level is characterized by a tumor H-score may be a median tumor H-score in a group of subjects with the same disease (e.g., cancer) or may be a value within a range above or below the median. In some embodiments, the reference level may be ≤30%, ≤25%, ≤20%, ≤15%, ≤10%, or ≤5% above or below the median. In various embodiments, the reference level may be 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300.

In some embodiments, CD73 expression level is characterized relative to tumor area. For example, in some embodiments CD73 expression level may be characterized by the number of CD73-positive tumor cells showing positive staining at any intensity and/or the number of CD73-positive tumor-associated immune cells showing positive staining at any intensity and/or CD73-positive stromal cells or other cells of the tumor microenvironment showing positive staining at any intensity relative to tumor area. In some embodiments, positive staining refers to cells (tumor or immune) showing partial or complete membrane staining at any intensity that is distinct from cytoplasmic staining. In some embodiments, positive staining refers to cells (tumor or immune) showing partial or complete membrane staining at any intensity and/or cytoplasmic staining. Tumor-associated immune cells may be intra- and peri-tumoral, including those present within the tumor proper, between tumor nests, and within any tumor-associated reactive stroma. In lymph nodes with focal or discrete tumor metastases, immune cells immediately adjacent to the leading edge of the metastatic tumor nest are typically defined as tumor-associated immune cells. The reference level when CD73 expression level is characterized relative to tumor area may be a median score in a group of subjects with the same disease (e.g., cancer) or may be a value within a range above or below the median. In some embodiments, the reference level may be ≤30%, ≤25%, ≤20%, ≤15%, ≤10%, or ≤5% above or below the median.

In some embodiments, a biomarker of the present disclosure is a cytokine expression level (e.g., CCL4, CD163, CXCL9, CXCL10, CXCL11, IFNγ, IL-6, IL-12B, IL-18, NGAL, TNFα, etc.) greater than or equal to a reference level or lower than a reference level. In some embodiments, cytokine expression levels are detected in a tumor sample. In some embodiments, cytokine expression levels are detected in a blood sample (e.g., whole blood, plasma, or serum). In some embodiments, a biomarker of the present disclosure is CXCL9 expression level, CXCL10 expression level, IL-6 expression level, or IFNγ expression level, TNFα expression level, or any combination thereof, measured in a blood sample, greater than or equal to a reference level. In some embodiments, a biomarker of the present disclosure is CXCL9 expression level or CXCL10 expression level, measured in a blood sample, greater than or equal to a reference level. In some embodiments, a biomarker of the present disclosure is CXCL11 expression level, IL-12B expression level, IL-18 expression level, or TNFα expression level, or any combination thereof, measured in a blood sample, less than a reference level. When cytokine protein levels are detected by an immunoassay, for example using an antibody that specifically binds to the cytokine in either a singleplex format or a multiplex format, or by proximity extension assays, aptamer-based assays, or mass spectrometry, detectable expression may be influenced by the sensitivity of the method. Exemplary methods for characterizing cytokine expression levels by immunoassay and establishing reference values are described in Example 8. The numerical value for a given cut-off can change depending on the method used to measure the cytokine and the timepoint the sample was collected. Alternative methods for measuring cytokines are known in the art (e.g., Guan et al., Nature, 2024, 627:646-655) and can be adapted to the disclosures herein.

In some embodiments, a biomarker of the present disclosure is CXCL9, CXCL10, IL-6 or IFNγ expression level, or any combination thereof, in a blood sample, that is greater than or equal to a reference level. In some embodiments, the reference level is a median value (e.g., concentration) for a group of subjects with the same disease (e.g., cancer) or the reference level is a value within a range above or below the median. In some embodiments, the reference level may be ≤30%, ≤25%, ≤20%, ≤15%, ≤10%, or ≤5% above or below the median. In some embodiments, the reference level may be ≤15%, ≤10%, or ≤5% above or below the median. In some embodiments, expression levels are characterized by an immunoassay.

In some embodiments, a biomarker of the present disclosure is CXCL11, IL-12B, IL-18, or TNFα expression levels, or any combination thereof, in a blood sample that is less than a reference level. In some embodiments, the reference level is a median value (e.g., concentration) for a group of subjects with the same disease (e.g., cancer) or the reference level is a value within a range above or below the median. In some embodiments, the reference level may be ≤30%, ≤25%, ≤20%, ≤15%, ≤10%, or ≤5% above or below the median. In some embodiments, the reference level may be ≤15%, ≤10%, or ≤5% above or below the median. In some embodiments, expression levels are characterized by an immunoassay.

In some embodiments, the blood sample is obtained prior to the initiation of treatment (e.g., a baseline sample). High baseline CXCL9 and/or high baseline CXCL10 compared to low baseline expression may be associated with poor clinical outcomes (e.g., poor survival) in patients receiving PD-(L)1 antagonist therapy (e.g., a treatment with an anti-PD-(L)1 antibody±chemotherapy). In contrast, high baseline CXCL9 and/or high baseline CXCL10 may predict additional benefit with a TIGIT antagonist (e.g., an anti-TIGIT antibody) over treatment with a PD-(L)1 antagonist. Accordingly, the present disclosure provides for the use of a combination comprising a TIGIT antagonist and a PD-(L)1 antagonist±chemotherapy to treat cancer in a patient with high baseline CXCL9 and/or high baseline CXCL10. In some embodiments, high baseline CXCL9 is an amount of CXCL9 greater than or equal to a median value derived from samples obtained from a group of patients known to have the same cancer, or is an amount of CXCL9 greater than or equal to a value about 30%, about 25%, about 20%, about 15%, about 10%, or about 5% above or below the median. In some embodiments, high baseline CXCL9 is an amount of CXCL9 greater than or equal to a median value derived from samples obtained from a group of patients known to have the same cancer, or is an amount of CXCL9 greater than or equal to a value about 15%, about 10%, or about 5% above or below the median. In some embodiments, high baseline CXCL10 is an amount of CXCL10 greater than or equal to a median value derived from samples obtained from a group of patients known to have the same cancer, or is an amount of CXCL10 greater than or equal to a value about 30%, about 25%, about 20%, about 15%, about 10%, or about 5% above or below the median. In some embodiments, high baseline CXCL10 is an amount of CXCL10 greater than or equal to a median value derived from samples obtained from a group of patients known to have the same cancer, or is an amount of CXCL10 greater than or equal to a value about 15%, about 10%, or about 5% above or below the median.

In some embodiments, cytokine levels are measured in two or more samples, e.g., a baseline sample and a second blood sample obtained after one or more treatment cycles (e.g., about 1, 2, 3, 4, 5, 6, or 7 days, or about 1, 2, 3, 4, 5, 6, 7, 8, 9, 10 or more weeks after a first treatment). A change in the amount of cytokine in the second sample as compared to the first sample is referred to herein as “an induction” or “a post-treatment increase.” The term “high induction” means the increase is equal to or greater than a reference value. The term “low induction” means the change is less than a reference value. Low induction of IL-18 expression levels, low induction of IL-12B expression levels, high induction of CXCL9 expression levels, high induction of CXCL10 expression levels, or high induction of IFNγ expression levels (or combinations thereof) in blood following treatment with a PD-(L)1 antagonist (e.g., an anti-PD-(L)1 antibody)±chemotherapy is associated with poor clinical outcomes (e.g., low response rates, short PFS, etc.) for patients with cancer. In contrast, low induction of IL-18 expression levels, low induction of IL-12B expression levels, high induction of CXCL9 expression levels, high induction of CXCL10 expression levels, or high induction of IFNγ expression levels, or any combination thereof, in blood following combination therapy comprising a TIGIT antagonist (e.g., an anti-TIGIT antibody) and a PD-(L)1 antagonist (e.g., an anti-PD-(L)1 antibody)±chemotherapy may predict clinical benefit. Accordingly, in some embodiments the present disclosure provides for the continued use of a combination comprising a TIGIT antagonist and a PD-(L)1 antagonist±chemotherapy to treat to cancer in a patient with low induction of IL-18 expression levels, low induction of IL-12B expression levels, high induction of CXCL9 expression levels, high induction of CXCL10 expression levels, or high induction of IFNγ expression levels, or any combination thereof, after treatment (e.g., one or more cycles) with the combination. In some embodiments, the reference level is a median fold-change derived from samples obtained from a group of patients known to have the same cancer, wherein the samples were obtained at approximately the same time before treatment and approximately the same time after the first treatment.

In the above embodiments, the sample can be obtained from a subject with cancer, optionally a solid tumor. In embodiments where a biomarker is used to select a therapy, expression of the biomarker is measured or was measured in a sample obtained prior to administration of the therapy. In some embodiments, the sample is obtained from a subject with a cancer other than lung cancer, e.g., an upper GI cancer, e.g., gastric, gastroesophageal junction (GEJ) or esophageal adenocarcinoma EAC, e.g., resectable gastric, GEJ, or EAC (e.g., Stage II or Stage III gastric, GEJ, or EAC), e.g., locally advanced unresectable gastric, GEJ or EAC (e.g., Stage III gastric, GEJ or EAC), or recurrent or metastatic gastric, GEJ or EAC (e.g., Stage IV gastric, GEJ or EAC)). In some embodiments, the sample is obtained from a subject with lung cancer, e.g., NSCLC, e.g., squamous or non-squamous NSCLC, e.g., resectable NSCLC (e.g., Stage II or Stage III NSCLC), e.g., locally advanced unresectable NSCLC (e.g., Stage III NSCLC), or recurrent or metastatic NSCLC (e.g., Stage IV NSCLC)).

Optimal biomarker cut-offs (e.g., reference values) can be analyzed using the median values described above, or using quartiles or tertiles, as reference points. Statistical methods, including but not limited to receiver operating characteristic (ROC) analysis and those described in the examples, can also be used to determine the cut-off of best fit, whereby the difference in clinical benefit between biomarker-high and biomarker-low populations is most significant.

### METHODS AND USES

In one aspect, the present disclosure is directed to uses of biomarkers described herein. The biomarkers described herein are useful in a variety of methods, including but not limited to methods of identifying a patient for treatment with a therapy, methods of treating a disease in a patient (e.g., a patient in need thereof), methods of determining a prognosis for a patient who has been identified as having or as at risk for a disease or condition (e.g., cancer), and methods for determining a prognosis for subject or a patient, such as a subject or a patient who has received or is yet to receive a therapy (e.g., a therapy comprising an immune checkpoint inhibitor, e.g., a PD-(L)1 antagonist, a TIGIT antagonist, or a PD-(L)1 antagonist and a TIGIT antagonist).

In certain embodiments, a biomarker is assessed, observed, measured, analyzed, or characterized in a sample obtained from a patient. The sample can be a sample comprising tumor cells. The sample can be a sample comprising immune cells. The sample can be a sample comprising tumor cells and immune cells. How to obtain such as sample is well known in the art. In particular, the sample can be obtained by biopsy or resection.

The sample to be analyzed in the context of the methods of the present disclosure may be obtained prior, during, or after treatment of a disease (e.g., cancer) as described herein. In embodiments where a biomarker is used to select a therapy, expression of the biomarker is measured or was measured in a sample obtained prior to treatment (e.g., administration of a therapy). A sample obtained prior to treatment can be obtained not more than one year, not more than six, five, four, three or two months, or one month prior to the initiation of said treatment. It is also contemplated to obtain a sample not more than two weeks, or not more than one week prior to said treatment. A sample obtained after treatment can be obtained after the end of the treatment. A sample obtained after treatment can be obtained not more than three years, obtained not more one year, not more than six, five, four, three or two months, or one month after said treatment. It is also contemplated to obtain a sample not more than two weeks, or not more than one week after said treatment.

Determining the amount of the biomarkers referred to in this specification relates to measuring a detectable level in a biological sample, preferably qualitatively or quantitatively. Measuring can be done directly or indirectly. Direct measuring relates to measuring the biomarker based on a signal which is obtained from the biomarker itself, and the intensity of which directly correlates with the number of molecules of the polypeptide present in the sample. Such a signal—sometimes referred to herein as intensity signal—may be obtained, e.g., by measuring an intensity value of a specific physical or chemical property of the biomarker. Indirect measuring includes measuring a signal obtained from a secondary component (i.e., a component not being the biomarker itself) or a biological read-out system, e.g., measurable cellular responses, ligands, labels, or enzymatic reaction products.

In accordance with the present disclosure, determining the amount of a biomarker can be achieved by all known means for determining the amount of a biomarker in a sample. Non-limiting examples of suitable means for detecting protein and nucleic acid biomarkers are described above. Said means comprise immunoassay devices and methods which may utilize labeled molecules in various sandwich, competition, or other assay formats. For example, when the biomarker comprises a protein, the immunoassay can be immunohistochemistry (IHC), immunocytochemistry, immunofluorescence microscopy, or immunophenotyping by flow cytometry, as described elsewhere herein. When the biomarker comprises a nucleic acid, the immunoassay can be fluorescence in situ hybridization (FISH).

The term “amount” as used herein encompasses an absolute amount of a biomarker, a relative amount of a biomarker, as well as any value or parameter which correlates thereto or can be derived therefrom. Such values or parameters comprise intensity signal values from all specific physical or chemical properties obtained from the said biomarker by direct measurements. Moreover, encompassed are all values or parameters which are obtained by indirect measurements specified elsewhere in this description. It is to be understood that values correlating to the aforementioned amounts or parameters can also be obtained by all standard mathematical operations.

In some embodiments, an amount or level (e.g., expression level) of a biomarker is compared to a reference level. The term “comparing” as used herein encompasses comparing the amount of the biomarker comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount, while a concentration is compared to a reference concentration, or an intensity signal obtained from a test sample is compared to the same type of intensity signal of a reference sample. The comparison may be carried out manually or computer assisted. For a computer assisted comparison, the value of the determined amount may be compared to values corresponding to suitable references which are stored in a database by a computer program. The computer program may further evaluate the result of the comparison, e.g., automatically provide the desired assessment in a suitable output format. Based on the comparison, it is possible to predict one or more of a patient's likelihood of responding to a therapy described herein, or a patient's prognosis before or after receiving a therapy described herein. The comparison also allows for selecting or identifying a candidate for a particular therapy described herein.

The term “reference level” as used herein refers to amounts of the biomarker(s) which allow for predicting whether a patient or subject is likely to respond to a therapy described herein, is a suitable candidate to receive a therapy described herein, and/or has a favorable or poor prognosis before or following a therapy described herein. In some embodiments, the reference level may be derived from a group of subjects known to have a particular disease (e.g., cancer, type of cancer, type of cancer and stage of cancer, etc.) that is the same as the patient in question. For clarity, this group of subjects is “unselected” with respect to said biomarker(s). Preferably, said reference level is derived from samples obtained from the aforementioned subjects (i.e., the reference level is a predetermined value). In some embodiments, a reference level can be a median expression level, or an amount ≤30%, ≤25%, ≤20%, ≤15%, ≤10%, or ≤5% above or below a median expression level, derived from samples obtained from a group of subjects known to have the same disease. In some embodiments, a reference level can be an amount ≤30%, ≤25%, ≤20%, ≤15%, ≤10%, or ≤5% above a median expression level derived from samples obtained from a group of subjects known to have the same disease. In some embodiments, a reference level can be an amount ≤30%, ≤25%, ≤20%, ≤15%, ≤10%, or ≤5% below a median expression level derived from samples obtained from a group of subjects known to have the same disease.

In the context of the methods of the present disclosure, the amount of more than one biomarker may be determined. For clarity, determined amounts shall be compared to various reference amounts, i.e., to a reference amount for the individual biomarker tested.

Moreover, the reference levels, preferably, define threshold amounts or thresholds. Suitable reference amounts or threshold amounts may be determined by the method of the present disclosure from a reference sample. The reference sample can be analyzed simultaneously with the test sample or can be analyzed before or after a test sample is analyzed. Once a threshold is established for a particular disease, comparison to a reference amount does not require reanalyzing a reference sample with each test sample. In some preferred embodiments, the reference level is a median expression level, or an amount ≤30%, ≤25%, ≤20%, ≤15%, ≤10%, or ≤5% above or below a median expression level, derived from samples obtained from a group of subjects known to have the same disease (e.g., same cancer, optionally same disease stage and in some embodiments same setting/line of therapy). In some embodiments, the reference level is an amount about 30% above or below a median expression level derived from samples obtained from group of subjects known to have the same disease (e.g., same cancer, optionally same disease stage and in some embodiments same setting/line of therapy). In some embodiments, the reference level is an amount about 25% above or below a median expression level derived from samples obtained from a group of subjects known to have the same disease (e.g., same cancer, optionally same disease stage and in some embodiments same setting/line of therapy). In some embodiments, the reference level is an amount about 20% above or below a median expression level derived from samples obtained from a group subjects known to have the same disease (e.g., same cancer, optionally same disease stage and in some embodiments same setting/line of therapy). In some embodiments, the reference level is an amount about 15% above or below a median expression level derived from samples obtained from a group of subjects known to have the same disease (e.g., same cancer, optionally same disease stage and in some embodiments same setting/line of therapy). In some embodiments, the reference level is an amount about 10% above or below a median expression level derived from samples obtained from a group of subjects known to have the same disease (e.g., same cancer, optionally same disease stage and in some embodiments same setting/line of therapy). In some embodiments, the reference level is an amount about 5% above or below a median expression level derived from samples obtained from a group of subjects known to have the same disease (e.g., same cancer, optionally same disease stage and in some embodiments same setting/line of therapy).

### Immune Checkpoint Inhibitors and Combination Therapy

In various embodiments, the present disclosure contemplates the use of the biomarkers disclosed herein to affect treatment decisions. In embodiments directed to treating cancer, a treatment may comprise administering an effective amount of an immune checkpoint inhibitor. As used herein, the terms “immune checkpoint inhibitor” and “CPI” may be used interchangeably to refer to an antagonist of an inhibitory or co-inhibitory immune checkpoint. Immune checkpoint inhibitors may antagonize an inhibitory or co-inhibitory immune checkpoint by interfering with receptor-ligand binding and/or altering receptor signaling. Examples of immune checkpoints (ligands and receptors), some of which are selectively upregulated in various types of cancer cells, that can be antagonized include PD-1; PD-L1 (PD-1 ligand); BTLA (B and T lymphocyte attenuator); CTLA-4 (cytotoxic T-lymphocyte associated antigen 4); TIM-3 (T cell immunoglobulin and mucin domain containing protein 3); LAG-3 (lymphocyte activation gene 3); TIGIT (T cell immunoreceptor with Ig and ITIM domains); CD276 (B7-H3); PD-L2; Galectin 9; CEACAM-1; CD69; Galectin-1; CD113; GPR56; VISTA; 2B4; CD48; GARP; PD1H; LAIR1; TIM-1; and TIM-4; and Killer Inhibitory Receptors.

In some embodiments, an immune checkpoint inhibitor is a CTLA-4 antagonist. In further embodiments, the CTLA-4 antagonist can be an antagonistic CTLA-4 antibody. Suitable antagonistic CTLA-4 antibodies include, for example, monospecific antibodies such as ipilimumab or tremelimumab, as well as bispecific antibodies such as MEDI5752 and KN046.

In some embodiments, an immune checkpoint inhibitor is a PD-1 antagonist that blocks an interaction between PD-1 and a ligand (e.g., PD-L1). In further embodiments, the PD-1 antagonist can be an antagonistic PD-1 antibody (“an anti-PD-1 antibody”), small molecule or peptide. Suitable antagonistic PD-1 antibodies include, for example, monospecific antibodies such as balstilimab, budigalimab, camrelizumab, cosibelimab, dostarlimab, cemiplimab, ezabenlimab (BI-754091), MEDI-0680 (AMP-514; WO2012/145493), nivolumab, pembrolizumab, pidilizumab (CT-011), pimivalimab, retifanlimab, sasanlimab, spartalizumab, sintilmab, tislelizumab, toripalimab, and zimberelimab; as well as bi-specific antibodies such as LY3434172. In still further embodiments, the PD-1 antagonist can be a recombinant protein composed of the extracellular domain of PD-L2 (B7-DC) fused to the Fc portion of IgG1 (AMP-224).

In some embodiments, an immune checkpoint inhibitor is zimberelimab. In some embodiments, zimberelimab is administered at a dose of about 100 mg to about 600 mg or about 200 mg to about 600 mg, about 600 mg to about 800 mg. In some embodiments, zimberelimab is administered at a dose of about 100 mg, 150 mg, 200 mg, 220 mg, 240 mg, 260 mg, 300 mg, 320 mg, 340 mg, 360 mg, 380 mg, 400 mg, 420 mg, 440 mg, or 460 mg. Doses of zimberelimab may be administered once a week, or less frequently (e.g., once every two, three, four, five, six weeks, or more). In some embodiments, a dosing cycle comprises administering domvanalimab at a dose of about 360 mg once every three weeks or at a dose of about 480 mg once every four weeks.

In some embodiments, an immune checkpoint inhibitor is a PD-L1 antagonist that blocks an interaction between PD-L1 and PD-1. In further embodiments, the PD-L1 antagonist can be an antagonistic PD-L1 antibody (“an anti-PD-L1 antibody”). Suitable antagonistic PD-L1 antibodies include, for example, monospecific antibodies such as avelumab, atezolizumab, durvalumab, BMS-936559, and envafolimab as well as bi-specific antibodies such as LY3434172 and KN046.

In some embodiments, an immune checkpoint inhibitor is a TIGIT antagonist that blocks an interaction between TIGIT and CD155. In further embodiments, the TIGIT antagonist can be an antagonistic TIGIT antibody (“an anti-TIGIT antibody”). Suitable antagonistic TIGIT antibodies include but are not limited to monospecific antibodies such as AGEN1327, AB308 (WO2021247591), BAT6021, COM902, domvanalimab, belrestotug, etigilimab, IBI-939, JS006, PM1021, dargistotug, ociperlimab, renvistobart, SEA-TGT, tiragolumab, vibostolimab; as well as bi-specific antibodies such as AGEN1777, AZD2936, D3L-002, HB036, HLX301, KA-1874, PM1009, SHR02992, and SIM0348. In certain embodiments, an immune checkpoint inhibitor is an antagonistic TIGIT antibody disclosed in WO2017152088 or WO2021247591. In certain embodiments, an immune checkpoint inhibitor is domvanalimab or AB308.

In some embodiments, an immune checkpoint inhibitor is an anti-TIGIT antibody with reduced binding to one or more activating FcγRs, such as FcγR isotypes I, IIA, IIB, IIIA, and IIIB, as compared to a wildtype (WT) IgG1 control antibody. In some embodiments, an immune checkpoint inhibitor is an anti-TIGIT antibody that does not meaningfully bind FcγR isotypes I, IIA, IIB, IIIA, IIIB, or combinations thereof. Binding to FcγR isotypes I, IIA, IIB, IIIA, and IIIB can be measured, for example, by the methods detailed in Example 1 of WO2023215719A1. Alternatively, or in addition, reduced binding to an activating FcγR can be demonstrated by showing an anti-TIGIT antibody has reduced Fe effector function, such as reduced CDC, ADCC, and/or ADCP compared to a compared to a wildtype IgG1 control antibody. Suitable methods for evaluating CDC, ADCC, and/or ADCP can be measured, for example, by the methods detailed in Example 1 of WO2023215719A1.

In some embodiments, an immune checkpoint inhibitor is an Fc-silent anti-TIGIT antibody, i.e., an anti-TIGIT antibody with no meaningful binding to FcγR isotypes I, IIA, IIB, IIIA, and IIIB (in some instances no binding).

Means for engineering an antibody (e.g., a known anti-TIGIT antibody with a WT hIgG1 Fc domain) to have reduced binding (including no binding) to activating FcγRs, such as FcγR isotypes I, IIA, IIB, IIIA, and IIIB, are known in the art. One approach can be to use different human IgG isotypes which naturally have reduced FcγR interactions, such as hIgG2 or hIgG4, as well as variants thereof. Additionally, Fc mutations have also been described to achieve the same purpose. Substitution of any or all of positions 234, 235, 236 and/or 237 reduces affinity for Fcγ receptors, particularly FcγRI receptor (see, e.g., U.S. Pat. No. 6,624,821). Alanine is a preferred residue for substitution and L234A/L235A is a preferred dual mutation to reduce Fc effector function. Other combinations of mutations with reduced Fc effector functions include L234A/L235A/G237A, E233P/L234V/L235A/AG236, A327G/A330S/P331S, K322A, L234A and L235A, L234F/L235E/P331S. Optionally, positions 234, 236 and/or 237 in human IgG2 are substituted with alanine and position 235 with glutamine. (see, e.g., U.S. Pat. No. 5,624,821.) Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 of IgG2 residues at positions 233-236 and IgG4 residues at positions 327, 330 and 331 greatly reduces ADCC and CDC (see, for example, Armour K L. Et al., 1999 Eur J Immunol. 29(8):2613-24; and Shields R L. et al., 2001. J Biol Chem. 276(9):6591-604). N297A, N297Q, or N297G (Eu numbering) mutations reduce glycosylation and thereby Fc effector functions. Other substitutions can also be made in the constant regions of antibodies of the present disclosure to reduce Fc effector function such as complement-mediated cytotoxicity or ADCC (see, e.g., Winter et al., U.S. Pat. No. 5,624,821; Tso et al., U.S. Pat. No. 5,834,597; Lazar et al., Proc. Natl. Acad. Sci. USA, 103:4005, 2006; and Schlothauer et al., Protein Engineering, Design, and Selection, 29(10): 457-466, 2016).

In some embodiments, an Fc-silent anti-TIGIT antibody is domvanalimab. Domvanalimab has an engineered IgG1 Fc and reduced binding to one or more activating FcγRs as compared to WT hIgG1. See Example 1 of WO2023215719A1. Domvanalimab was tested by enzyme-linked immunosorbent assay for binding to FcγR isotypes I, IIA, JIB, IIIA, and IIIB. When compared with a wild-type IgG1 control antibody, no significant binding was observed with domvanalimab for any FcγR isotype when tested up to a maximum concentration of 1 M. Domvanalimab was also tested in an FcγR-IIIA (V158 high-affinity variant) effector reporter bioassay and found to be inactive at concentrations up to 1 M. A CDC assay was performed with human complement and a Jurkat cell line stably overexpressing human TIGIT in the presence of domvanalimab at increasing concentrations up to 33 nM. No cytotoxicity was seen for domvanalimab at any concentration tested.

In some embodiments, an Fc-silent anti-TIGIT antibody is ASP8374, COM902, JS006, or renvistobart.

In some embodiments, an Fc-silent anti-TIGIT antibody is a variant of a known anti-TIGIT antibody comprising a wildtype human IgG1 Fc domain or a human IgG1 Fc domain engineered to have enhanced Fc effector function (e.g., AGEN1327, AGEN1777, AZD2936, BAT6021, D3L-002, HB036, HLX301, IBI-939, KA-1874, PM1009, PM1021, SEA-TGT, SHR02992, SIM0348, belrestotug, etigilimab, dargistotug, ociperlimab, ralzapastotug, tiragolumab, vibostolimab, etc.). In these embodiments, the variant comprises one or more Fc mutations (such as those described above) that reduce binding to FcγR isotypes I, IIA, IIB, IIIA, and IIIB. Typically the variant will comprise the same (or substantially the same) light chain and heavy chain variable regions as the anti-TIGIT antibody from which it was derived.

In some embodiments, an immune checkpoint inhibitor is domvanalimab. In some embodiments, domvanalimab is administered at a dose of about 500 mg to about 2000 mg, about 800 mg to about 1600 mg, about 600 mg to about 800 mg, about 900 mg to about 1200 mg, about 1200 mg to about 1600 mg. Doses of domvanalimab may be administered once a week, or less frequently (e.g., once every two, three, four, five, six weeks, or more). In some embodiments, a dosing cycle comprises administering domvanalimab at a dose of about 1200 mg once every three weeks or at a dose of about 1600 mg once every four weeks.

In some embodiments, an immune checkpoint inhibitor is a LAG-3 antagonist. In further embodiments, the LAG-3 antagonist can be an antagonistic LAG-3 antibody (“an anti-LAG-3 antibody”). Suitable antagonistic LAG-3 antibodies include, for example, fianlimab, BMS-986016 (WO10/19570, WO14/08218), or IMP-731 or IMP-321 (WO08/132601, WO09/44273).

In certain embodiments, an immune checkpoint inhibitor is a B7-H3 antagonist. In further embodiments, the B7-H3 antagonist is an antagonistic B7-H3 antibody (“an anti-B7-H3 antibody”). Suitable antagonist B7-H3 antibodies include, for example, enoblituzumab (MGA271; WO11/109400), omburtumab, DS-7300a, ABBV-155, and SHR-A1811.

In some embodiments, an immune checkpoint inhibitor is a TIM-3 antagonist. In further embodiments, the TIM-3 antagonist can be an antagonistic TIM-3 antibody (“an anti-TIM-3 antibody”). Suitable antagonistic TIM-3 antibodies include, for example, dostarlimab, sabatolimab, BMS-986258, and RG7769/RO7121661.

Depending upon the disease (e.g., type of cancer), the immune checkpoint inhibitor may be used as a monotherapy or may be used in combination with one or more additional therapy. When used in combination, each additional therapy can be a therapeutic agent or another treatment modality. Selection of the additional therapies may be informed by current standard of care for a particular cancer and/or mutational status of a subject's cancer and/or stage of disease. Detailed standard of care guidelines are published, for example, by National Comprehensive Cancer Network (NCCN). See, for instance, NCCN Colon Cancer v2.2023, NCCN Hepatobiliary Cancer v1.2023, NCCN Kidney Cancer, v4.2023, NCCN NSCLC v3.2023, NCCN Pancreatic Adenocarcinoma v1.2023, NCCN Esophageal and Esophagogastric Junction Cancers v2.2023, NCCN Gastric Cancer v1.2023, Cervical Cancer v1.2023, Ovarian Cancer/Fallopian Tube Cancer/Primary Peritoneal Cancer v1.2023, Hepatocellular Carcinoma v1.2023.

In embodiments comprising one or more additional treatment modality, the immune checkpoint inhibitor can be administered before, after or during treatment with the additional treatment modality. Non-limiting examples of additional treatment modalities include surgical resection of a tumor, bone marrow transplant, radiation therapy, and photodynamic therapy. In embodiments comprising one or more additional therapeutic agent, the therapeutic agents used in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a patient's course of therapy. If the combination is formulated as separate compositions, in some embodiments, the separate compositions are provided together in a kit.

In some embodiments, one or more of the additional therapies is an additional treatment modality. Exemplary treatment modalities include but are not limited to surgical resection of a tumor, bone marrow transplant, radiation therapy, and photodynamic therapy.

In some embodiments, one or more of the additional therapies is a therapeutic agent. Exemplary therapeutic agents include chemotherapeutic agents, radiopharmaceuticals, hormone therapies, epigenetic modulators, ATP-adenosine axis-targeting agents, targeted therapies, signal transduction inhibitors, RAS signaling inhibitors, PI3K inhibitors, arginase inhibitors, HIF inhibitors, AXL inhibitors, PAK4 inhibitors, immunotherapeutic agents, cellular therapies, gene therapies, immune checkpoint inhibitors, and agonists of stimulatory or co-stimulatory immune checkpoints.

In some embodiments, one or more of the additional therapeutic agents is a chemotherapeutic agent. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, pomalidomide, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pemetrexed, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab paclitaxel, and docetaxel; chlorambucil; gemcitabine; 6-thioguanine; mercaptopurine; methotrexate; platinum and platinum coordination complexes (i.e., “platinum-containing chemotherapeutic agent”) such as cisplatin, carboplatin and oxaliplatin; vinca alkaloids such as vinblastine, vincristine, vindesine, vinorelbine; etoposide (VP-16); ifosfamide; mitomycin C; mitoxantrone; vincristine; vinorelbine; navelbine; novantrone; teniposide; daunomycin; aminopterin; xeloda; ibandronate; CPT11; proteasome inhibitors such as bortezomib, carfilzomib and ixazomib; topoisomerase inhibitors such as irinotecan, SN-38, topotecan, etoposide, mitoxantrone, teniposide; difluoromethylornithine (DMFO); retinoic acid; esperamicins; capecitabine; anthracyclines and pharmaceutically acceptable salts, acids or derivatives of any of the above. In certain embodiments, combination therapy comprises chemotherapy that includes one or more chemotherapeutic agents. In one embodiment, combination therapy comprises chemotherapy comprising one or more of FOLFOX (folinic acid, fluorouracil, and oxaliplatin), FOLFIRI (e.g., folinic acid, fluorouracil, and irinotecan), CAPOX (capecitabine and oxaliplatin), a taxoid (e.g., docetaxel, paclitaxel, nab-paclitaxel, etc.), NALIRIFOX (fluorouracil, leucovorin, liposomal irinotecan, and oxaliplatin), a fluoropyrimidine-containing chemotherapeutic agent (e.g., fluorouracil, capecitabine, floxuridine), a platinum-containing chemotherapeutic agent, a topoisomerase inhibitor, and/or gemcitabine.

In some embodiments, one or more of the additional therapeutic agents is a radiopharmaceutical. A radiopharmaceutical is a form of internal radiation therapy in which a source of radiation (i.e., one or more radionuclide) is put inside a subject's body. The radiation source can be in solid or liquid form. Non-limiting examples of radiopharmaceuticals include sodium iodide I-131, radium-223 dichloride, lobenguane iodine-131, radioiodinated vesicles (e.g., saposin C-dioleoylphosphatidylserine (SapC-DOPS) nanovesicles), various forms of brachytherapy, and various forms of targeted radionuclides. Targeted radionuclides comprise a radionuclide associated (e.g., by covalent or ionic interactions) with a molecule (“a targeting agent”) that specifically binds to a target on a cell, typically a cancer cell or an immune cell. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen (i.e., an antigen enriched but not specific to a cancer cell), a tumor-specific antigen (i.e., an antigen with minimal to no expression in normal tissue), or a neo-antigen (i.e., an antigen specific to the genome of a cancer cell generated by non-synonymous mutations in the tumor cell genome). Non-limiting examples of targeted radionuclides include radionuclides attached to: somatostatin or peptide analogs thereof (e.g., 177Lu-Dotatate, etc.); prostate specific membrane antigen or peptide analogs thereof (e.g., 177Lu-PSMA-617, 225Ac-PSMA-617, 177Lu-PSMA-I&T, 177Lu-MIP-1095, etc.); a receptor's cognate ligand, peptide derived from the ligand, or variants thereof (e.g., 188Re-labeled VEGF₁₂₅₋₁₃₆ or variants thereof with higher affinity to VEGF receptor, etc.); antibodies targeting tumor antigens (e.g., 1311-tositumomab, 90Y-ibritumomab tiuxetan, CAM-H2-I131 (Precirix NV), 1131-omburtamab, etc.).

In some embodiments, one or more of the additional therapeutic agents is a hormone therapy. Hormone therapies act to regulate or inhibit hormonal action on tumors. Examples of hormone therapies include, but are not limited to: selective estrogen receptor degraders such as fulvestrant, giredestrant, SAR439859, RG6171, AZD9833, rintodestrant, ZN-c5, LSZ102, D-0502, LY3484356, SHR9549; selective estrogen receptor modulators such as tamoxifen, raloxifene, 4-hydroxytamoxifen, trioxifene, keoxifene, toremifene; aromatase inhibitors such as anastrozole, exemestane, letrozole and other aromatase inhibiting 4(5)-imidazoles; gonadotropin-releasing hormone agonists such as nafarelin, triptorelin, goserelin; gonadotropin-releasing hormone antagonists such as degarelix; antiandrogens such as abiraterone, enzalutamide, apalutamide, darolutamide, flutamide, nilutamide, bicalutamide, leuprolide; 5α-reductase inhibitors such as finasteride, dutasteride; and the like. In certain embodiments, combination therapy comprises administration of a hormone or related hormonal agent. In one embodiment, combination therapy comprises administration of enzalutamide.

In some embodiments, one or more of the additional therapeutic agents is an epigenetic modulator. An epigenetic modulator alters an epigenetic mechanism controlling gene expression, and may be, for example, an inhibitor or activator of an epigenetic enzyme. Non-limiting examples of epigenetic modulators include DNA methyltransferase (DNMT) inhibitors, hypomethylating agents, and histone deacetylase (HDAC) inhibitors. In one or more embodiments, an immune checkpoint inhibitor is combined with DNMT inhibitors or hypomethylating agents. Exemplary DNMT inhibitors include decitabine, zebularine and azacitadine. In one or more embodiments, combinations of a CPI with a HDAC inhibitor are also contemplated. Exemplary HDAC inhibitors include vorinostat, givinostat, abexinostat, panobinostat, belinostat and trichostatin A.

In some embodiments, one or more of the additional therapeutic agents is an ATP-adenosine axis-targeting agent. ATP-adenosine axis-targeting agents alter signaling mediated by adenine nucleosides and nucleotides (e.g., adenosine, AMP, ADP, ATP), for example by modulating the level of adenosine or targeting adenosine receptors. In some embodiments, an ATP-adenosine axis-targeting agent is an inhibitor of an ectonucleotidase involved in the conversion of ATP to adenosine or an antagonist of adenosine receptor (e.g., CD39 or CD73). Exemplary small molecule CD73 inhibitors include CB-708, ORIC-533, LY3475070 and quemliclustat. Exemplary anti-CD39 and anti-CD73 antibodies include ES002023, TTX-030, IPH-5201, SRF-617, CPI-006, oleclumab, NZV930, IPH5301, GS-1423, uliledlimab, AB598, and BMS-986179. In some embodiments, an ATP-adenosine axis-targeting agent is an A₂ₐR antagonist, an A_{2b}R antagonist, or an antagonist of A₂ₐR and A_{2b}R. Exemplary adenosine receptor inhibitors include etrumadenant, inupadenant, taminadenant, caffeine citrate, NUV-1182, TT-702, DZD-2269, INCB-106385, EVOEXS-21546, AZD-4635, imaradenant, RVU-330, ciforadenant, PBF-509, PBF-999, PBF-1129, and CS-3005. In some embodiments, the present disclosure contemplates the combination of a CPI described herein with etrumadenant, quemliclustat, AB598, or a combination thereof.

In some embodiments, one or more of the additional therapeutic agents is a targeted therapy. In one aspect, a targeted therapy may comprise targeting agent and a drug. The drug may be a chemotherapeutic agent, a radionuclide, a hormone therapy, or another small molecule drug attached to a targeting agent. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen, a tumor-specific antigen, or a neo-antigen. In some embodiments, the targeted therapy is an antibody-drug conjugate comprising an antibody and a drug, wherein the antibody specifically binds to HER2, HER3, nectin-4, or Trop-2. Specific examples of a targeted therapy comprising an antibody and a drug include but are not limited to patritumab deruxtecan, sacituzumab govitecan-hziy, telisotuzumab vedotin, and trastuzumab deruxtecan. In other aspects, a targeted therapy may inhibit or interfere with a specific protein that helps a tumor grow and/or spread. Non-limiting examples of such targeted therapies include signal transduction inhibitors, RAS signaling inhibitors, inhibitors of oncogenic transcription factors, activators of oncogenic transcription factor repressors, angiogenesis inhibitors, immunotherapeutic agents, ATP-adenosine axis-targeting agents, AXL inhibitors, PARP inhibitors, PAK4 inhibitors, PI3K inhibitors, HIF-2a inhibitors, CD39 inhibitors, CD73 inhibitors, A2R antagonists, TIGIT antagonists, and PD-(L)1 antagonists.

In some embodiments, one or more of the additional therapeutic agents is a signal transduction inhibitor. Signal transduction inhibitors are agents that selectively inhibit one or more steps in a signaling pathway. Signal transduction inhibitors (STIs) contemplated by the present disclosure include but are not limited to: (i) BCR-ABL kinase inhibitors (e.g., imatinib); (ii) epidermal growth factor receptor tyrosine kinase inhibitors (EGFR TKIs), including small molecule inhibitors (e.g., CLN-081, gefitinib, erlotinib, afatinib, icotinib, and osimertinib), and anti-EGFR antibodies; (iii) inhibitors of the human epidermal growth factor (HER) family of transmembrane tyrosine kinases, e.g., HER-2/neu receptor inhibitors (e.g., trastuzumab) and HER-3 receptor inhibitors; (iv) vascular endothelial growth factor receptor (VEGFR) inhibitors including small molecule inhibitors (e.g., axitinib, regorafenib, sunitinib and sorafenib), VEGF kinase inhibitors (e.g., lenvatinib, cabozantinib, pazopanib, tivozanib, XL092, etc.) anti-VEGF antibodies (e.g., bevacizumab), and anti-VEGFR antibodies (e.g., ramucirumab); (v) inhibitors of AKT family kinases or the AKT pathway (e.g., rapamycin); (vi) inhibitors of mTOR, such as, for example, everolimus, sirolimus, temsirolimus; (vii) inhibitors of serine/threonine-protein kinase B-Raf (BRAF), such as, for example, vemurafenib, dabrafenib and encorafenib; (viii) inhibitors of rearranged during transfection (RET), including, for example, selpercatinib and pralsetinib; (ix) tyrosine-protein kinase Met (MET) inhibitors (e.g., tepotinib, tivantinib, cabozantinib and crizotinib); (x) anaplastic lymphoma kinase (ALK) inhibitors (e.g., ensartinib, ceritinib, lorlatinib, crizotinib, and brigatinib); (xi) inhibitors of the RAS signaling pathway (e.g., inhibitors of KRAS, HRAS, RAF, MEK, ERK) as described elsewhere herein; (xii) FLT-3 inhibitors (e.g., gilteritinib); (xiii) inhibitors of Trop-2; (xiv) inhibitors of the JAK/STAT pathway, e.g., JAK inhibitors including tofacitinib and ruxolitinib, or STAT inhibitors such as napabucasin; (xv) inhibitors of NF-kB; (xvi) cell cycle kinase inhibitors (e.g., flavopiridol); (xvii) phosphatidyl inositol kinase (PI3K) inhibitors; (xiii) protein kinase B (AKT) inhibitors (e.g., capivasertib, miransertib); (xx) platelet-derived growth factor receptor (PDGFR) inhibitors (e.g., imatinib, sunitinib, regorafenib, avapritinib, lenvatinib, nintedanib, famitinib, ponatinib, axitinib, repretinib, etc.); (xix) insulin-like growth factor receptor (IGFR) inhibitors (e.g., erlotinib, afatinib, gefitinib, osimertinib, dacomitinib); (xx) fibroblast growth factor receptor (FGFR) inhibitors (e.g., futibatinib, erdafitinib, pemigatinib); and (xxi) receptor tyrosine kinase KIT inhibitors (e.g., imatinib, sorafenib, sunitinib, masitinib, repretinib, avapritinib). In one or more embodiments, the additional therapeutic agent comprises an inhibitor of EGFR, VEGFR, HER-2, HER-3, BRAF, RET, MET, ALK, RAS (e.g., KRAS, MEK, ERK), FLT-3, JAK, STAT, NF-kB, PI3K, AKT, FGFR, KIT, or any combinations thereof.

In some embodiments, one or more of the additional therapeutic agents is a RAS signaling inhibitor. Oncogenic mutations in the RAS family of genes, e.g., HRAS, KRAS, and NRAS, are associated with a variety of cancers. For example, mutations of G12C, G12D, G12V, G12A, G13D, Q61H, G13C and G12S, among others, in the KRAS family of genes have been observed in multiple tumor types. Direct and indirect inhibition strategies have been investigated for the inhibition of mutant RAS signaling. Indirect inhibitors target effectors other than RAS in the RAS signaling pathway, and include, but are not limited to, inhibitors of RAF, MEK, ERK, PI3K, PTEN, SOS (e.g., SOS1), mTORC1, SHP2 (PTPN11), and AKT. Non-limiting examples of indirect inhibitors under development include RMC-4630, RMC-5845, RMC-6291, RMC-6236, JAB-3068, JAB-3312, TNO155, RLY-1971, BI1701963. Direct inhibitors of RAS mutants have also been explored, and generally target the KRAS-GTP complex or the KRAS-GDP complex. Exemplary direct RAS inhibitors under development include, but are not limited to, sotorasib, adagrasib, mRNA-5671 and ARS1620. In some embodiments, the one or more RAS signaling inhibitors are selected from the group consisting of RAF inhibitors, MEK inhibitors, ERK inhibitors, PI3K inhibitors, PTEN inhibitors, SOS1 inhibitors, mTORC1 inhibitors, SHP2 inhibitors, and AKT inhibitors. In other embodiments the one or more RAS signaling inhibitors directly inhibit RAS mutants.

In some embodiments, one or more of the additional therapeutic agents is a VEGF inhibitor or a VEGFR inhibitor. In some embodiment, the VEGF or VEGFR inhibitor is a small molecule VEGFR inhibitor, a small molecule VEGF kinase inhibitor, an anti-VEGF antibody, or an anti-VEGFR antibody. In some embodiments, the present disclosure contemplates the combination of a CPI described herein with axitinib, bevacizumab, cabozantinib, lenvatinib, pazopanib, ramucirumab, regorafenib, sunitinib, sorafenib, tivozanib, or XL092. In some embodiments, the present disclosure contemplates the combination of a CPI described herein with axitinib, cabozantinib, lenvatinib, pazopanib, regorafenib, sunitinib, sorafenib, tivozanib, or XL092.

In some embodiments, one or more of the additional therapeutic agents is an inhibitor of a hypoxia-inducible factor (HIF) transcription factor, particularly HIF-2a. Exemplary HIF-2a inhibitors include belzutifan, ARO-HIF2, PT-2385, and those described in WO 2021113436, WO 2021188769, and WO 2023077046. In some embodiments, the present disclosure contemplates the combination of a CPI described herein with AB521.

In some embodiments, one or more of the additional therapeutic agents is an inhibitor of anexelekto (AXL). Exemplary multikinase AXL inhibitors include sitravatinib, rebastinib, glesatinib, gilteritinib, merestinib, cabozantinib, foretinib, BMS777607, LY2801653, S49076, and RXDX-106. AXL specific inhibitors have also been developed, e.g., small molecule inhibitors including DS-1205, SGI-7079, SLC-391, dubermatinib, bemeentinib, AB801, and DP3975; anti-AXL antibodies such as ADCT-601; and antibody drug conjugates (ADCs) such as BA3011. Another strategy to inhibit AXL signaling involves targeting AXL's ligand, GAS6. For example, batiraxcept is under development as is a Fe fusion protein that binds the GAS6 ligand thereby inhibiting AXL signaling. In some embodiments a CPI is combined with one or more AXL inhibitors described in WO 2022246177 or WO 2022246179. In some embodiments, the AXL inhibitor is AB801.

In some embodiments, one or more of the additional therapeutic agents is an immunotherapeutic agent. Immunotherapeutic agents treat a disease by stimulating or suppressing the immune system. Immunotherapeutic agents useful in the treatment of cancers typically elicit or amplify an immune response to cancer cells. Non-limiting examples of suitable immunotherapeutic agents include: immunomodulators (e.g., cytokines, chemokines, etc.); cellular immunotherapies (e.g., CAR-T cell therapy, CAR-NK cell therapy, TCR therapy, dendritic cell vaccines, etc.); vaccines; gene therapies; ATP-adenosine axis-targeting agents; immune checkpoint modulators; and certain signal transduction inhibitors.

In some embodiments, one or more of the additional therapeutic agents is an immune checkpoint inhibitor. Suitable immune checkpoint inhibitors are described above.

In some embodiments, one or more of the additional therapeutic agents activates a stimulatory or co-stimulatory immune checkpoint. Examples of stimulatory or co-stimulatory immune checkpoints (ligands and receptors) include B7-1, B7-2, CD28, 4-1BB (CD137), 4-1BBL, ICOS, ICOS-L, OX40, OX40L, GITR, GITRL, CD70, CD27, CD40, DR3 and CD2.

In some embodiments, one or more of the additional therapeutic agents is an immunotherapeutic agent, more specifically an intracellular signaling molecules that influences immune cell function. For example, one or more of the additional therapies may be an inhibitor of hematopoietic progenitor kinase 1 (HPK1). HPK1 is serine/threonine kinase that functions as a negative regulator of activation signals generated by the T cell antigen receptor. As another example, one or more of the additional therapies may be an inhibitor of Cbl-b, an E3 ubiquitin ligase involved in the regulation of TCR signaling. As another example, one or more of the additional therapies may be an inhibitor of diacylglycerol kinase (DGK). In some embodiments, the inhibitor is a small molecule. Non-limiting examples of small molecule HPK1 inhibitors in clinical development include NDI-101150, PRJ1-3024, PF-07265028, GRC 54276, CFI-402411 and BGB-15025; non-limiting examples of Cbl-b inhibitors in clinical development include AP401, HST-1011, and NX-1607. Non-limiting examples of small molecule DAG inhibitors include ASP1570, and BAY2965501.

In some embodiments, each additional therapy can independently be chemotherapy, a radiopharmaceutical, a hormone therapy, an epigenetic modulator, a targeted agent, an immunotherapeutic agent, a cellular therapy, or a gene therapy. For example, in one embodiment, the present disclosure contemplates the use of a CPI (e.g., a PD-(L)1 antagonist, a TIGIT antagonist, or a PD-(L)1 antagonist and a TIGIT antagonist) in combination with chemotherapy and optionally one or more additional therapeutic agents, wherein each additional therapeutic agent is independently a radiopharmaceutical, a hormone therapy, a targeted agent, an immunotherapeutic agent, a cellular therapy, or a gene therapy. In another embodiment, the present disclosure contemplates the use of a CPI (e.g., a PD-(L)1 antagonist, a TIGIT antagonist, or a PD-(L)1 antagonist and a TIGIT antagonist) in combination with chemotherapy and optionally one or more additional therapeutic agents, wherein each additional therapeutic agent is independently a targeted agent, an immunotherapeutic agent, or a cellular therapy. In another embodiment, the present disclosure contemplates the use of a CPI (e.g., a PD-(L)1 antagonist, a TIGIT antagonist, or a PD-(L)1 antagonist and a TIGIT antagonist) in combination with one or more immunotherapeutic agents and optionally one or more additional therapeutic agent or therapy, wherein each additional therapeutic agent or therapy is independently a radiopharmaceutical, a hormone therapy, a targeted agent, chemotherapy, a cellular therapy, or a gene therapy. In another embodiment, the present disclosure contemplates the use of a CPI (e.g., a PD-(L)1 antagonist, a TIGIT antagonist, or a PD-(L)1 antagonist and a TIGIT antagonist) in combination with one or more immunotherapeutic agents and optionally one or more additional therapies, wherein each additional therapy is independently chemotherapy, a targeted agent, or a cellular therapy. In another embodiment, the present disclosure contemplates the use of a CPI (e.g., a PD-(L)1 antagonist, a TIGIT antagonist, or a PD-(L)1 antagonist and a TIGIT antagonist) in combination with one or more immune checkpoint inhibitors and/or one or more ATP-adenosine axis-targeting agents, and optionally one or more additional therapies, wherein each additional therapeutic agent is independently a chemotherapy, a targeted agent, an immunotherapeutic agent, or a cellular therapy. In further embodiments of the above (a) the targeted agent is a VEGF or VEGFR inhibitor (e.g., axitinib, bevacizumab, cabozantinib, lenvatinib, pazopanib, ramucirumab, regorafenib, sunitinib, sorafenib, tivozanib, or XL092), a PI3K inhibitor, an arginase inhibitor, a HIF2α inhibitor, an AXL inhibitor, or a PAK4 inhibitor; (b) the immunotherapeutic agent is an ATP-adenosine axis-targeting agent or an immune checkpoint inhibitor; (c) the ATP-adenosine axis-targeting agent is an A₂ₐR and/or A_{2b}R antagonist, a CD73 inhibitor, or a CD39 inhibitor; (d) the ATP-adenosine axis-targeting agent is etrumadenant, quemliclustat, or AB598; (e) the immunotherapeutic agent is an immune checkpoint inhibitor, optionally an anti-TIGIT antagonist antibody, optionally an Fc-silent antibody, optionally domvanalimab, (f) the chemotherapy is a platinum-containing chemotherapy, a platinum doublet, platinum- and fluoropyrimidine-based chemotherapy, pemetrexed and platinum chemotherapy, a taxane or taxane-containing chemotherapy, a topoisomerase inhibitor (e.g., irinotecan, SN-38, doxorubicin, etc.), carboplatin and either paclitaxel or paclitaxel protein-bound, FOLFOX, FOLFIRI, or CAPOX; or (g) any combination thereof. In still further embodiments of the above, the present disclosure contemplates the use of a CPI (e.g., a PD-(L)1 antagonist) in combination with anti-TIGIT antibody (e.g., an Fc-silent anti-TIGIT antibody, optionally domvanalimab, etc). In still further embodiments of the above, the present disclosure contemplates the use of a CPI (e.g., a PD-(L)1 antagonist) in combination with anti-TIGIT antibody (e.g., domvanalimab, etc.) and chemotherapy. In still further embodiments of the above, the present disclosure contemplates the use of a CPI (e.g., a PD-(L)1 antagonist) in combination with anti-TIGIT antibody (e.g., an Fc-silent anti-TIGIT antibody, optionally domvanalimab, etc.) and a VEGF or VEGFR inhibitor. In still further embodiments of the above, the present disclosure contemplates the use of a CPI (e.g., a PD-(L)1 antagonist) in combination with domvanalimab, etrumadenant, quemliclustat, AB308, AB521, AB801, or any combination thereof. In some embodiments, the PD-(L)1 antagonist is atezolizumab, avelumab, cemiplimab, dostarlimab, durvalumab, nivolumab, pembrolizumab, retifanlimab, tislelizumab, toripalimab, or zimberelimab. In certain embodiments, the PD-(L)1 antagonist is zimberelimab.

### Methods for Identifying a Patient for Therapy

In one aspect, the present disclosure provides methods for identifying a patient for treatment with a therapy, such as a therapy comprising an immune checkpoint inhibitor. In some embodiments, provided is a method for identifying a suitable candidate for a particular therapy described herein. In some embodiments, provided is a method for selecting a patient for a particular therapy described herein. In some embodiments, the patient is a patient having cancer, such as a solid tumor. The biomarkers of the below embodiments are further described elsewhere herein, and those disclosures are incorporated by reference into this section. Suitable samples are also described elsewhere herein and incorporated by reference into this section.

In one aspect, the present disclosure provides biomarkers useful for identifying a patient for treatment with a therapy (e.g., a therapy comprising an immune checkpoint inhibitor, e.g., a PD-(L)1 antagonist, a TIGIT antagonist, or a PD-(L)1 antagonist and a TIGIT antagonist). In some embodiments, the biomarker is selected from PD-L1 (PD-L1 expression level), CD155 (CD155 expression level), CD226 (CD226 expression level), CD73 (CD73 expression level), an adenosine pathway biomarker, and any combination thereof. In some embodiments, the adenosine pathway biomarker is CD73 (CD73 expression level). A patient may be identified for treatment with a therapy comprising an immune checkpoint inhibitor when a sample obtained from the patient comprises: (a) a PD-L1 expression level greater than or equal to a PD-L1 reference level, or lower than a PD-L1 reference level; (b) a CD155 expression level greater than or equal to a CD155 reference level, or lower than a CD155 reference level; (c) a CD226 expression level greater than or equal to a CD226 reference level, or lower than a CD226 reference level; (d) an adenosine pathway biomarker expression level greater than or equal to an adenosine pathway biomarker reference level or lower than an adenosine pathway biomarker reference level; (e) a CD73 expression level greater than or equal to a CD73 reference level, or lower than a CD73 reference level, or (f) any combination of (a) to (e). Stated differently, the sample has been obtained and has been determined to comprise (a)-(f) via methods described herein. In some embodiments, the patient is identified for treatment with a therapy comprising an immune checkpoint inhibitor when (a) the PD-L1 expression level is greater than or equal to a PD-L1 reference level; (b) the CD155 expression level is lower than a CD155 reference level; (c) the CD226 expression level is greater than or equal to a CD226 reference level; (d) the adenosine pathway biomarker expression level is lower than an adenosine pathway biomarker reference level; (e) the CD73 expression level is lower than a reference level, or (f) any combination of (a) to (e). The sample can be a sample comprising tumor cells, a sample comprising immune cells (e.g., lymphocytes, monocytes, myeloid cells, dendritic cells, plasma cells, or combinations thereof), or a sample comprising both tumor cells and immune cells. The sample can also further comprise one or more additional cell type, e.g., another cell type in the tumor microenvironment.

In some embodiments, the therapy for which a patient is identified for treatment is a monotherapy. The monotherapy can include, without limitation an anti-PD-L1 antagonist or an anti-PD-1 antagonist (designated herein as “anti-PD-(L)1 antagonist”). The monotherapy can include, without limitation an anti-PD-L1 antibody or an anti-PD-1 antibody (designated herein as “anti-PD-(L)1 antibody”). In some embodiments, treatment with an anti-PD-(L)1 as a monotherapy may be indicated when CD155 expression level is lower than a CD155 reference level and/or CD73 expression level is greater than or equal to a CD73 reference level.

In some embodiments, the therapy for which a patient is identified for treatment is a combination therapy, as described herein. In some embodiments, the combination therapy comprises at least two therapies (i.e., 2, 3, 4, 5, or more therapies). In some embodiments, the combination therapy comprises at least three therapies (i.e., 3, 4, 5, 6, or more therapies). In some embodiments, the combination can include an anti-PD-1 antibody. In some embodiments, the combination can include an anti-PD-L1 antibody. In some embodiments, the combination can include an anti-TIGIT antibody. In some embodiments, the combination can include an anti-PD-1 antibody and an anti-TIGIT antibody. In some embodiments, the combination can include an anti-PD-L1 antibody and an anti-TIGIT antibody. In embodiments comprising an anti-TIGIT antibody, the anti-TIGIT antibody may be an Fc-silent anti-TIGIT antibody. In some embodiments, the combination can include an anti-PD-(L)1 antibody and one or more than one additional therapy, provided the combination does not include an anti-TIGIT antibody. Treatment with a combination therapy comprising an anti-TIGIT antibody may provide greater clinical benefit when CD73 expression level is less than a CD73 reference level and/or CD155 expression level is greater than or equal to a CD155 reference level, whereas treatment with a combination therapy that does not include an anti-TIGIT antibody but does include a different CPI (e.g., an anti-PD-(L)1 antibody) may provide greater clinical benefit when CD73 expression level is greater than or equal to a CD73 reference level and/or CD155 expression level is lower than a CD155 reference level.

In some embodiments, the combination can include an ATP-adenosine axis-targeting agent. In some embodiments, the combination does not include an ATP-adenosine axis-targeting agent. In some embodiments, the combination can include an antagonistic anti-PD-1 antibody, an antagonistic anti-TIGIT antibody, and an ATP-adenosine axis-targeting agent. In some embodiments, the combination can include an antagonistic anti-PD-L1 antibody, an antagonistic anti-TIGIT antibody, and an ATP-adenosine axis-targeting agent. In some embodiments, the ATP-adenosine axis-targeting agent is an adenosine receptor antagonist. In some embodiments, the ATP-adenosine axis-targeting agent is an A₂ₐR and/or A_{2b}R antagonist, a CD73 inhibitor, or a CD39 inhibitor. In some embodiments, the ATP-adenosine axis-targeting agent is an A₂ₐR and/or A_{2b}R antagonist.

In some embodiments, a combination therapy comprises chemotherapy, such as a chemotherapy agent or a chemotherapy regimen described herein. In some embodiments, the chemotherapy comprises a platinum-containing agent. In some embodiments, a combination therapy comprises a VEGF or VEGFR inhibitor. In some embodiments, a combination therapy comprises one or more additional therapy described elsewhere herein.

In some embodiments, a patient to be identified for a therapy described herein has cancer comprising a solid tumor. In some embodiments, the patient has a solid tumor selected from the group consisting of ovarian cancer, endometrial cancer, breast cancer, lung cancer, colon cancer, prostate cancer, cervical cancer, biliary cancer, pancreatic cancer, gastric cancer, esophageal cancer, liver cancer, kidney cancer, head-and-neck tumors, mesothelioma, melanoma, sarcomas, central nervous system (CNS) hemangioblastomas, and brain tumors. In some embodiments, the patient has a cancer selected from the group consisting of gastrointestinal cancer, genitourinary cancer, gynecological cancer, and lung cancer. In some embodiments, the patient has lung cancer, such as, without limitation, squamous cell lung cancer or non-squamous cell lung cancer (NSCLC). In some embodiments, the patient has upper gastrointestinal (GI) cancer, such as, without limitation, gastric cancer, gastroesophageal junction (GEJ) cancer, or esophageal adenocarcinoma (EAC). The cancer may be early-stage cancer (e.g., Stage I or Stage II). The cancer may be a locally advanced unresectable cancer. The cancer may be recurrent or metastatic cancer.

In some embodiments, a biomarker expression level in a sample is determined by immunohistochemistry (IHC). Expression level can be measured or determined based on IHC staining intensity and/or percentage of staining-positive cells (e.g., tumor cells and/or immune cells and/or stromal cells or other cells of the tumor microenvironment). In some embodiments, a reference level is determined by IHC. In some embodiments, the IHC staining intensity and/or the percentage of staining-positive cells is determined based on cytoplasmic staining, membrane staining, or both cytoplasmic and membrane staining. Methods of using IHC to determine the level of a biomarker in a sample are known in the art and further described herein.

In some embodiments, a reference level is a median expression level, or an amount about 30%, about 25%, about 20%, about 15%, about 10%, or about 5% above or below a median expression level, derived from samples obtained from a group of subjects known to have the same disease. For example, in some embodiments, a PD-L1 reference level can be an amount about 30%, about 25%, about 20%, about 15%, about 10%, or about 5% above or below a median PD-L1 expression level derived from samples obtained from a group of patients known to have the same disease. In some embodiments, a CD155 reference level can be an amount about 30%, about 25%, about 20%, about 15%, about 10%, or about 5% above or below a median CD155 expression level derived from samples obtained from patients of a group of patients known to have the same disease. In some embodiments, a CD226 reference level can be an amount about 30%, about 25%, about 20%, about 15%, about 10%, or about 5% above or below a median CD226 expression level derived from samples obtained from a group of patients known to have the same disease. In some embodiments, an adenosine pathway biomarker reference level can be an amount about 30%, about 25%, about 20%, about 15%, about 10%, or about 5% above or below a median adenosine pathway biomarker expression level derived from samples obtained from a group subjects known to have the same disease. In some embodiments, a CD73 reference level can be an amount about 30%, about 25%, about 20%, about 15%, about 10%, or about 5% above or below a median CD73 expression level derived from samples obtained from a group of patients known to have the same disease.

In some embodiments, a patient is identified for treatment with a therapy comprising an immune checkpoint inhibitor (e.g., a TIGIT antagonist, or a TIGIT antagonist in combination with a PD-(L)1 antagonist) when, in a sample obtained from the patient: (a) a PD-L1 expression level is greater than or equal to a median PD-L1 expression level or greater than or equal to an amount ≤30%, ≤25%, ≤20%, ≤15%, ≤10%, or ≤5% above or below a median PD-L1 expression level derived from samples obtained from a group of patients known to have the same disease; (b) a CD155 expression level is greater than or equal to a median CD155 expression level or greater than or equal to an amount ≤30%, ≤25%, ≤20%, ≤15%, ≤10%, or ≤5% above or below a median CD155 expression level derived from samples obtained from a group of patients known to have the same disease; (c) a CD226 expression level is greater than or equal to a median CD226 expression level or greater than or equal to an amount ≤30%, ≤25%, ≤20%, ≤15%, ≤10%, or ≤5% above or below a median CD226 expression level derived from samples obtained from a group of patients group known to have the same disease; (d) an adenosine pathway biomarker expression level is lower than a median adenosine pathway biomarker expression level or lower than an amount ≤30%, ≤25%, ≤20%, ≤15%, ≤10%, or ≤5% above or below a median adenosine pathway biomarker expression level derived from samples obtained from a group of patients known to have the same disease; (e) a CD73 expression level is lower than a median CD73 expression level or lower than an amount ≤30%, ≤25%, ≤20%, ≤15%, ≤10%, or ≤5% above or below a median CD73 expression level derived from samples obtained from a group of patients group known to have the same disease; or (f) any combination of (a) to (e).

In some embodiments, a patient is identified for treatment with a therapy comprising an immune checkpoint inhibitor (e.g., a TIGIT antagonist, or a TIGIT antagonist in combination with a PD-(L)1 antagonist) when, in a sample obtained from the patient: (a) a PD-L1 expression level is greater than or equal to a median PD-L1 expression level or greater than or equal to an amount ≤30%, ≤25%, ≤20%, ≤15%, ≤10%, or ≤5% above or below a median PD-L1 expression level derived from samples obtained from a group of patients known to have the same disease; (b) a CD155 expression level is greater than or equal to a median CD155 expression level or greater than or equal to an amount ≤30%, ≤25%, ≤20%, ≤15%, ≤10%, or ≤5% above or below a median CD155 expression level derived from samples obtained from a group of patients known to have the same disease; (c) a CD226 expression level is greater than or equal to a median CD226 expression level or greater than or equal to an amount ≤30%, ≤25%, ≤20%, ≤15%, ≤10%, or ≤5% above or below a median CD226 expression level derived from samples obtained from a group of patients known to have the same disease; (d) a CD73 expression level is lower than a median CD73 expression level or lower than an amount ≤30%, ≤25%, ≤20%, ≤15%, ≤10%, or ≤5% above or below a median CD73 expression level derived from samples obtained from a group of patients known to have the same disease; or (e) any combination of (a) to (d).

In some embodiments, a patient is identified for treatment with a therapy comprising an immune checkpoint inhibitor (e.g., a TIGIT antagonist, or a TIGIT antagonist in combination with a PD-(L)1 antagonist) when, in a sample obtained from the patient: (a) is PD-L1 high (e.g., TC ≥50% for NSCLC or TAP ≥5% for EAC, GEJ, gastric or other cancers) or is PD-L1 positive (e.g., TC ≥1% for NSCLC or TAP ≥1% for EAC, GEJ, gastric or other cancers); (b) a CD155 expression level is greater than or equal to a median CD155 expression level or greater than or equal to an amount ≤30%, ≤25%, ≤20%, ≤15%, ≤10%, or ≤5% above or below a median CD155 expression level derived from samples obtained from a group of patients known to have the same disease; (c) a CD226 expression level is greater than or equal to a median CD226 expression level or greater than or equal to an amount ≤30%, ≤25%, ≤20%, ≤15%, ≤10%, or ≤5% above or below a median CD226 expression level derived from samples obtained from a group of patients known to have the same disease; (d) a CD73 expression level is lower than a median CD73 expression level or lower than an amount ≤30%, ≤25%, ≤20%, ≤15%, ≤10%, or ≤5% above or below a median CD73 expression level derived from samples obtained from a group of patients known to have the same disease; or (e) any combination of (a) to (d). In some embodiments, the therapy comprises an anti-TIGIT antibody. In some embodiments, the therapy comprises an anti-PD-(L)1 antibody and an anti-TIGIT antibody. In some embodiments, the anti-TIGIT antibody is an Fc-silent anti-TIGIT antibody.

In some embodiments, expression levels may be expressed in numerous ways, including but not limited to biomarker-positive tumor cell fraction (“% TC”), biomarker-positive tumor cell fraction staining at medium or high intensity (“2+ or 3+ % TC”), biomarker-positive immune cell fraction (“% IC”), characterization by a tumor H-score, and characterization of expression relative to tumor area (e.g., the number of biomarker-positive tumor cells showing positive staining at any intensity and/or the number of biomarker-positive tumor-associated immune cells showing positive staining at any intensity relative to tumor area), as described elsewhere herein.

### Methods of Treatment

The present disclosure provides methods for treating a disease (e.g., cancer) in patient known to have the disease, the method comprising administering an effective amount of a therapy to the patient when the patient is determined to have a particular pattern of expression of one or more biomarkers. The biomarkers of the below embodiments are further described elsewhere herein, and those disclosures are incorporated by reference into this section. Suitable samples are also described elsewhere herein and incorporated by reference into this section.

In one aspect, the present disclosure provides methods for treating a patient with an effective amount of a therapy (e.g., a therapy comprising an immune checkpoint inhibitor, e.g., a PD-(L)1 antagonist, a TIGIT antagonist, or a PD-(L)1 antagonist and a TIGIT antagonist). In some embodiments, the therapy is administered to the patient when a sample obtained from the patient comprises a particular expression pattern of one or more biomarkers described herein. In some embodiments, the biomarker is selected from PD-L1 (PD-L1 expression level), CD155 (CD155 expression level), CD226 (CD226 expression level), an adenosine pathway biomarker, CD73 (CD73 expression level), and any combination thereof. In some embodiments, the adenosine pathway biomarker is CD73 (CD73 expression level). A patient may be treated with a therapy comprising an immune checkpoint inhibitor, as described herein, when a sample obtained from the patient comprises: (a) a PD-L1 expression level greater than or equal to a PD-L1 reference level, or lower than a PD-L1 reference level; (b) a CD155 expression level greater than or equal to a CD155 reference level, or lower than a CD155 reference level; (c) a CD226 expression level greater than or equal to a CD226 reference level, or lower than a CD226 reference level; (d) an adenosine pathway biomarker expression level greater than or equal to an adenosine pathway biomarker reference level, or lower than an adenosine pathway biomarker reference level; (e) a CD73 expression level greater than or equal to a CD73 reference level, or lower than a CD73 reference level, or (f) any combination of (a) to (e). Stated differently, the sample has been obtained and has been determined to comprise (a)-(f) via methods described herein. In some embodiments, the patient is identified for treatment with a therapy comprising an immune checkpoint inhibitor when (a) the PD-L1 expression level is greater than or equal to a PD-L1 reference level; (b) the CD155 expression level is lower than a CD155 reference level; (c) the CD226 expression level is greater than or equal to a CD226 reference level; (d) the adenosine pathway biomarker expression level is lower than an adenosine pathway biomarker reference level; (e) the CD73 expression level is lower than a CD73 reference level, or (f) any combination of (a) to (e). In some embodiments, the patient is identified for treatment with a therapy comprising an immune checkpoint inhibitor (e.g., a TIGIT antagonist, or a TIGIT antagonist in combination with a PD-(L)1 antagonist) when the CD155 expression level is greater than or equal to a CD155 reference level, and optionally (a) the PD-L1 expression level is greater than or equal to a PD-L1 reference level; (b) the CD226 expression level is greater than or equal to a CD226 reference level; (c) the adenosine pathway biomarker expression level is lower than an adenosine pathway biomarker reference level; (d) the CD73 expression level is lower than a CD73 reference level, or (f) any combination of (a) to (d). The sample can be a sample comprising tumor cells, a sample comprising immune cells (e.g., lymphocytes, monocytes, myeloid cells, dendritic cells, plasma cells, or any combination thereof), or a sample comprising both tumor cells and immune cells.

In some embodiments, the therapy administered to the patient is a monotherapy. The monotherapy can include, without limitation an anti-PD-L1 antagonist or an anti-PD-1 antagonist. The monotherapy can include, without limitation an anti-PD-L1 antibody or an anti-PD-1 antibody. In some embodiments, treatment with an anti-PD-(L)1 as a monotherapy may be indicated when CD155 expression level is lower than a CD155 reference level and/or CD73 expression level is greater than or equal to a CD73 reference level.

In some embodiments, the therapy administered to the patient is a combination therapy, as described herein. In some embodiments, the combination therapy comprises at least two therapies (i.e., 2, 3, 4, 5, or more therapies). In some embodiments, the combination therapy comprises at least three therapies (i.e., 3, 4, 5, 6, or more therapies). In some embodiments, the combination can include an anti-PD-1 antibody. In some embodiments, the combination can include an anti-PD-L1 antibody. In some embodiments, the combination can include an anti-TIGIT antibody. In some embodiments, the combination can include an anti-PD-1 antibody and an anti-TIGIT antibody. In some embodiments, the combination can include an anti-PD-L1 antibody and an anti-TIGIT antibody. In embodiments comprising an anti-TIGIT antibody, the anti-TIGIT antibody may be an Fc-silent anti-TIGIT antibody. In some embodiments, the combination can include an anti-PD-(L)1 antibody and one or more than one additional therapy, provided the combination does not include an anti-TIGIT antibody. Treatment with a combination therapy comprising an anti-TIGIT antibody may provide greater clinical benefit when CD73 expression level is less than a CD73 reference level and/or when CD155 expression level is greater than or equal to a CD155 reference level, whereas treatment with a combination therapy that does not include an anti-TIGIT antibody but does include a different CPI (e.g., an anti-PD-(L)1 antibody) may provide greater clinical benefit when CD73 expression level is greater than or equal to a CD73 reference level and/or CD155 expression level is lower than a CD155 reference level.

In some embodiments, the combination can include an ATP-adenosine axis-targeting agent. In some embodiments, the combination can include an anti-PD-1 antibody, an anti-TIGIT antibody, and an ATP-adenosine axis-targeting agent. In some embodiments, the combination can include an anti-PD-L1 antibody, an anti-TIGIT antibody, and an ATP-adenosine axis-targeting agent. In some embodiments, the combination can include an anti-PD-(L)1 antibody, an anti-TIGIT antibody, and an ATP-adenosine axis-targeting agent. In some embodiments, the ATP-adenosine axis-targeting agent is an adenosine receptor antagonist. In some embodiments, the ATP-adenosine axis-targeting agent is an A₂ₐR and/or A_{2b}R antagonist, a CD73 inhibitor, or a CD39 inhibitor. In some embodiments, the ATP-adenosine axis-targeting agent is an A₂ₐR and/or A_{2b}R antagonist.

In some embodiments, a combination therapy comprises chemotherapy, such as a chemotherapy agent or a chemotherapy regimen described herein. In some embodiments, the chemotherapy comprises a platinum-containing agent. In some embodiments, a combination therapy comprises a VEGF or VEGFR inhibitor. In some embodiments, a combination therapy comprises one or more additional therapy described elsewhere herein.

In some embodiments, a patient to be administered a therapy described herein has cancer comprising a solid tumor. In some embodiments, the patient has a solid tumor selected from the group consisting of ovarian cancer, endometrial cancer, breast cancer, lung cancer, colon cancer, prostate cancer, cervical cancer, biliary cancer, pancreatic cancer, gastric cancer, esophageal cancer, liver cancer, kidney cancer, head-and-neck tumors, mesothelioma, melanoma, sarcomas, central nervous system (CNS) hemangioblastomas, and brain tumors. In some embodiments, the patient has a cancer selected from the group consisting of gastrointestinal cancer, genitourinary cancer, gynecological cancer, and lung cancer. In some embodiments, the patient has lung cancer, such as, without limitation, squamous cell lung cancer or non-squamous cell lung cancer (NSCLC). In some embodiments, the patient has upper gastrointestinal (GI) cancer, such as, without limitation, gastric cancer, gastroesophageal junction (GEJ) cancer, or esophageal adenocarcinoma (EAC). The cancer may be a locally advanced unresectable cancer. The cancer may be recurrent or metastatic cancer.

In some embodiments, a biomarker expression level in a sample is determined by immunohistochemistry (IHC). Expression level can be measured or determined based on IHC staining intensity and/or percentage of staining-positive cells (e.g., tumor cells and/or immune cells). In some embodiments, a reference level is determined by IHC. In some embodiments, the IHC staining intensity and/or the percentage of staining-positive cells is determined based on cytoplasmic staining, membrane staining, or both cytoplasmic and membrane staining. Methods of using IHC to determine the level of a biomarker in a sample are known in the art and further described herein.

In some embodiments, a reference level is a median expression level, or an amount about 30%, about 25%, about 20%, about 15%, about 10%, or about 5% above or below a median expression level, derived from samples obtained from a group patients known to have the same disease. For example, in some embodiments, a PD-L1 reference level can be an amount about 30%, about 25%, about 20%, about 15%, about 10%, or about 5% above or below a median PD-L1 expression level derived from samples obtained from a group of patients known to have the same disease. In some embodiments, a CD155 reference level can be an amount about 30%, about 25%, about 20%, about 15%, about 10%, or about 5% above or below a median CD155 expression level derived from samples obtained from a group of patients known to have the same disease. In some embodiments, a CD226 reference level can be an amount about 30%, about 25%, about 20%, about 15%, about 10%, or about 5% above or below a median CD226 expression level derived from samples obtained from a group of patients known to have the same disease. In some embodiments, an adenosine pathway biomarker reference level can be an amount about 30%, about 25%, about 20%, about 15%, about 10%, or about 5% above or below a median adenosine pathway biomarker expression level derived from samples obtained from a group of patients known to have the same disease. In some embodiments, a CD73 reference level can be an amount about 30%, about 25%, about 20%, about 15%, about 10%, or about 5% above or below a median CD73 expression level derived from samples obtained from a group of patients known to have the same disease.

In some embodiments, a patient is administered a therapy comprising an immune checkpoint inhibitor (e.g., a TIGIT antagonist, or a TIGIT antagonist in combination with a PD-(L)1 antagonist) when, in a sample obtained from the patient: (a) a PD-L1 expression level is greater than or equal to a median PD-L1 expression level or greater than or equal to an amount ≤30%, ≤25%, ≤20%, ≤15%, ≤10%, or ≤5% above or below a median PD-L1 expression level derived from samples obtained from a group of patients known to have the same disease; (b) a CD155 expression level is greater than or equal to a median CD155 expression level or greater than or equal to an amount ≤30%, ≤25%, ≤20%, ≤15%, ≤10%, or ≤5% above or below a median CD155 expression level derived from samples obtained from a group of patients known to have the same disease; (c) a CD226 expression level is greater than or equal to a median CD226 expression level or greater than or equal to an amount ≤30%, ≤25%, ≤20%, ≤15%, ≤10%, or ≤5% above or below a median CD226 expression level derived from samples obtained from a group of patients known to have the same disease; (d) an adenosine pathway biomarker expression level is lower than a median adenosine pathway biomarker expression level or lower than an amount ≤30%, ≤25%, ≤20%, ≤15%, ≤10%, or ≤5% above or below a median adenosine pathway biomarker expression level derived from samples obtained from a group of patients known to have the same disease; I a CD73 expression level is lower than a median CD73 expression level or lower than an amount ≤30%, ≤25%, ≤20%, ≤15%, ≤10%, or ≤5% above or below a median CD73 expression level derived from samples obtained from a group of patients known to have the same disease; or (f) any combination of (a) I(e).

In some embodiments, a patient is administered a therapy comprising an immune checkpoint inhibitor (e.g., a TIGIT antagonist, or a TIGIT antagonist in combination with a PD-(L)1 antagonist) when, in a sample obtained from the patient: (a) a PD-L1 expression level is greater than or equal to a median PD-L1 expression level or greater than or equal to an amount ≤30%, ≤25%, ≤20%, ≤15%, ≤10%, or ≤5% above or below a median PD-L1 expression level derived from samples obtained from a group of patients known to have the same disease; (b) a CD155 expression level is greater than or equal to a median CD155 expression level or greater than or equal to an amount ≤30%, ≤25%, ≤20%, ≤15%, ≤10%, or ≤5% above or below a median CD155 expression level derived from samples obtained from a group of patients known to have the same disease; (c) a CD226 expression level is greater than or equal to a median CD226 expression level or greater than or equal to an amount ≤30%, ≤25%, ≤20%, ≤15%, ≤10%, or ≤5% above or below a median CD226 expression level derived from samples obtained from a group of patients known to have the same disease; (d) a CD73 expression level is lower than a median CD73 expression level or lower than an amount ≤30%, ≤25%, ≤20%, ≤15%, ≤10%, or ≤5% above or below a median CD73 expression level derived from samples obtained from a group of patients known to have the same disease or (e) any combination of (a) to (d).

In some embodiments, expression levels may be expressed in numerous ways, including but not limited to biomarker-positive tumor cell fraction (“% TC”), biomarker-positive tumor cell fraction staining at medium or high intensity (“2+ or 3+ % TC”), biomarker-positive immune cell fraction (“% IC”), characterization by a tumor H-score, and characterization of expression relative to tumor area (e.g., the number of biomarker-positive tumor cells showing positive staining at any intensity and/or the number of biomarker-positive tumor-associated immune cells showing positive staining at any intensity relative to tumor area), as described elsewhere herein.

In one aspect, the present disclosure provides a method of treating a disease (e.g., cancer) in a patient known to have said disease, comprising (a) measuring in a sample obtained from a patient a level of one or more of PD-L1, CD155, CD226, an adenosine pathway biomarker, and CD73; (b) comparing the level measured in (a) to a respective reference level; and (c) administering to the patient an effective amount of a therapy comprising an immune checkpoint inhibitor when (i) a measured PD-L1 level is greater than or equal to a PD-L1 reference level or lower than a PD-L1 reference level, and/or (ii) a measured CD155 level is greater than or equal to a CD155 reference level or lower than a CD155 reference level, and/or (iii) a measured CD226 level is greater than or equal to a CD226 reference level or lower than a CD226 reference level, and/or (iv) a measured adenosine pathway biomarker level is greater than or equal to an adenosine pathway biomarker reference level or lower than an adenosine pathway biomarker reference level, and/or (v) a measured CD73 level is greater than or equal to a CD73 reference level or lower than a CD73 reference level. In some embodiments, the patient is administered an effective amount of the therapy when: (i) a measured PD-L1 level is greater than or equal to a PD-L1 reference level, and/or (ii) a measured CD155 level is greater than or equal to a CD155 reference level, and/or (iii) a measured CD226 level is greater than or equal to a CD226 reference level, and/or (iv) a measured adenosine pathway biomarker level is lower than an adenosine pathway biomarker reference level; and/or (v) a measured CD73 level is lower than a CD73 reference level. In some embodiments, the adenosine pathway biomarker is CD73 (CD73 expression level). In some of the aforementioned embodiments wherein the measured CD73 level is lower than a CD73 reference level, the therapy comprises a first immune checkpoint inhibitor that is not an anti-TIGIT antagonist, and a second immune checkpoint inhibitor that is an anti-TIGIT antagonist. In still further embodiments, the therapy comprises a PD-(L)1 antagonist, optionally an anti-PD-(L)1 antibody, and an anti-TIGIT antagonist, optionally an anti-TIGIT antibody. In some embodiments, the anti-TIGIT antagonist is an Fc-silent anti-TIGIT antibody.

In one aspect, the present disclosure provides a method of treating a CD73 low cancer in a patient comprising administering an effective amount of a therapy comprising an immune checkpoint inhibitor to the patient. In some embodiments, the therapy is combination therapy comprising a first immune checkpoint inhibitor that is not an anti-TIGIT antagonist, and a second immune checkpoint inhibitor that is an anti-TIGIT antagonist. In still further embodiments, the combination comprises a PD-(L)1 antagonist, optionally an anti-PD-(L)1 antibody, and an anti-TIGIT antagonist, optionally an anti-TIGIT antibody. In some embodiments, the anti-TIGIT antagonist is an Fc-silent anti-TIGIT antibody. In each of these embodiments, the combination therapy may further comprise one or more than one additional therapy. Suitable additional therapies are described elsewhere herein.

In some embodiments, a CD73 low cancer is a cancer in which CD73 expression level is below a threshold, wherein the threshold is a median CD73 expression level, or about 30%, about 25%, about 20%, about 15%, about 10%, or about 5% above or below a median CD73 expression level, derived from samples obtained from a group of patients known to have the same cancer (e.g., NSCLC, gastric cancer, GEJ, EAC, etc.). In some embodiments, an effective amount of the therapy is administered to the patient when it is determined that the patient has a CD73 low cancer. Stated differently, a sample has been obtained from the patient and has been determined to be CD73 via methods described herein. In some embodiments, the sample further comprises: (a) a PD-L1 expression level greater than or equal to a PD-L1 reference level; (b) a CD155 expression level greater than or equal to a CD155 reference level; (c) a CD226 expression level greater than or equal to a CD226 reference level; (d) any combination of (a) to (c). In some embodiments, an effective amount of the therapy is administered to the patient with a CD73 low cancer, when a sample obtained from the patient comprises: (a) a PD-L1 expression level is greater than or equal to a median PD-L1 expression level or greater than or equal to an amount ≤30%, ≤25%, ≤20%, ≤15%, ≤10%, or ≤5% above or below a median PD-L1 expression level derived from samples obtained from a group of patients known to have the same disease; (b) a CD155 expression level is greater than or equal to a median CD155 expression level or greater than or equal to an amount ≤30%, ≤25%, ≤20%, ≤15%, ≤10%, or ≤5% above or below a median CD155 expression level derived from samples obtained from a group of patients known to have the same disease; (c) a CD226 expression level is greater than or equal to a median CD226 expression level or greater than or equal to an amount ≤30%, ≤25%, ≤20%, ≤15%, ≤10%, or ≤5% above or below a median CD226 expression level derived from samples obtained from a group of known to have the same disease; or (d) any combination of (a) to (c).

In one aspect, the present disclosure provides a method of treating a CD155 high cancer in a patient comprising administering an effective amount of a therapy comprising an immune checkpoint inhibitor to the patient. In some embodiments, the therapy is combination therapy comprising a first immune checkpoint inhibitor that is not an anti-TIGIT antagonist, and a second immune checkpoint inhibitor that is an anti-TIGIT antagonist. In still further embodiments, the combination comprises a PD-(L)1 antagonist, optionally an anti-PD-(L)1 antibody, and an anti-TIGIT antagonist, optionally an anti-TIGIT antibody. In some embodiments, the anti-TIGIT antagonist is an Fc-silent anti-TIGIT antibody. In each of these embodiments, the combination therapy may further comprise one or more than one additional therapy. Suitable additional therapies are described elsewhere herein.

In some embodiments, a CD155 high cancer is a cancer in which CD155 expression level is above a threshold, wherein the threshold is a median CD155 expression level, or about 30%, about 25%, about 20%, about 15%, about 10%, or about 5% above or below a median CD155 expression level, derived from samples obtained from a group of patients known to have the same cancer (e.g., NSCLC, gastric cancer, GEJ, EAC, etc.). In some embodiments, an effective amount of the therapy is administered to the patient when it is determined that the patient has a CD155 high cancer. Stated differently, a sample has been obtained from the patient and has been determined to be CD155 high via methods described herein. In some embodiments, the sample further comprises: (a) a PD-L1 expression level greater than or equal to a PD-L1 reference level; (b) a CD73 expression level lower than or equal to a CD155 reference level; (c) a CD226 expression level greater than or equal to a CD226 reference level; (d) any combination of (a) to (c). In some embodiments, an effective amount of the therapy is administered to the patient with a CD155 high cancer, when a sample obtained from the patient comprises: (a) a PD-L1 expression level is greater than or equal to a median PD-L1 expression level or greater than or equal to an amount ≤30%, ≤25%, ≤20%, ≤15%, ≤10%, or ≤5% above or below a median PD-L1 expression level derived from samples obtained from a group of patients known to have the same disease; (b) a CD73 expression level is lower than or equal to a median CD73 expression level or greater than or equal to an amount ≤30%, ≤25%, ≤20%, ≤15%, ≤10%, or ≤5% above or below a median CD73 expression level derived from samples obtained from a group of patients known to have the same disease; (c) a CD226 expression level is greater than or equal to a median CD226 expression level or greater than or equal to an amount ≤30%, ≤25%, ≤20%, ≤15%, ≤10%, or ≤5% above or below a median CD226 expression level derived from samples obtained from a group of known to have the same disease; or (d) any combination of (a) to (c).

In some embodiments, the CD155 expression level for a sample is characterized by a CD155-positive tumor cell fraction (% TC). The reference level when CD155 expression level is characterized by CD155% TC may be a median CD155% TC in a group of subjects with the same disease (e.g., cancer) or may be a value within a range above or below the median. In some embodiments, the reference level may be ≤30%, ≤25%, ≤20%, ≤15%, ≤10%, or ≤5% above or below a median level. In various embodiments, the reference level may be 1% TC, 5% TC, 10% TC, 15% TC, 20% TC, 25% TC, 30% TC, 35% TC, 40% TC, 45% TC, 50% TC, 55% TC, 60% TC, 65% TC, 70% TC, 75% TC, 80% TC, 85% TC, or 90% TC.

In some embodiments, the CD155 expression level for a sample is characterized by a CD155 2+ or 3+ % TC. The reference level, when CD155 expression level is characterized by CD155 2+ or 3+ % TC, may be a median CD155 2+ or 3+ % TC in a group of subjects with the same disease (e.g., cancer) or may be a value within a range above or below the median. In some embodiments, the reference level may be ≤30%, ≤25%, ≤20%, ≤15%, ≤10%, or ≤5% above or below a median level. In some embodiments, the reference level may be 2+ or 3+ 30% TC, 2+ or 3+ 35% TC, 2+ or 3+ 40% TC, 2+ or 3+ 45% TC, 2+ or 3+ 50% TC, 2+ or 3+ 55% TC, 2+ or 3+ 60% TC, 2+ or 3+ 65% TC. In some embodiments, the reference level may be 2+ or 3+ 30% TC, 2+ or 3+ 35% TC, 2+ or 3+ 40% TC, 2+ or 3+ 45% TC, or 2+ or 3+ 50% TC. In some embodiments, the reference level may be 2+ or 3+ 40% TC, 2+ or 3+ 45% TC, 2+ or 3+ 50% TC, 2+ or 3+ 55% TC, 2+ or 3+ 60% TC. In some embodiments, the reference level may be 2+ or 3+ 50% TC. In some embodiments, the reference level may be 2+ or 3+ 7% TC, 2+ or 3+ 8% TC, 2+ or 3+ 9% TC, 2+ or 3+ 10% TC, 2+ or 3+ 11% TC, 2+ or 3+ 12% TC, or 2+ or 3+ 13% TC. In some embodiments, the reference level may be 2+ or 3+ 40% TC. In some embodiments, the reference level may be 2+ or 3+ 35% TC. In some embodiments, the reference level may be 2+ or 3+ 45% TC.

In some embodiments, the CD155 expression level for a sample is characterized by a tumor H-score. In some embodiments, the reference level may be ≤30%, ≤25%, ≤20%, ≤15%, ≤10%, or ≤5% above or below the median. In various embodiments, the reference level may be a tumor H-score of 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300. In some embodiments, the reference level may be a tumor H-score of 95, 100, 105, 110, 115, 120, 125, 130, 135, 140, 145, 150, 155, 160, 165, 170, or 175. In some embodiments, the reference level may be a tumor H-score of 110, 115, 120, 125, 130, 135, 140, 145, 150, 155, or 160. In some embodiments, the reference level may be a tumor H-score of 110, 115, 120, 125, 130, 135, 140, 145, or 150. In some embodiments, the reference level may be a tumor H-score of 120, 125, 130, 135, 140, 145, or 150. In some embodiments, the reference level may be a tumor H-score of 75, 80, 85, 90, 95, 100, 105, 110, 115, 120, 125, 130, or 135. In some embodiments, the reference level may be a tumor H-score of 120, 125, 130, 135, 140, 145, or 150. In some embodiments, the reference level may be a tumor H-score of 95, 100, 105, 110, 115, 120, 125, or 130. In some embodiments, the reference level may be a tumor H-score of 120, 125, 130, 135, 140, 145, or 150. In some embodiments, the reference level may be a tumor H-score of 75, 80, 85, 90, 95, 100, 105, 110, 115, 120, 125, 130, or 135. In some embodiments, the reference level may be a tumor H-score of 25, 30, 35, 40, 45, 50 or 55. In some embodiments, the reference level may be a tumor H-score of 95, 100, 105, 110, 115, 120, 125, or 130.

In some embodiments, the CD155 expression level for a sample is characterized by a cyto-membranous 2+ or 3+ % TC, in which the positive staining in the membrane and cytoplasm are combined. In some embodiments, the reference level may be ≤30%, ≤25%, ≤20%, ≤15%, ≤10%, or ≤5% above or below the median (e.g., 10% in gastric-intestinal cancers). In various embodiments, the reference level may be a cyto-membranous 2⁺ or 3+% TC of 8%, 9%, 10%, 11%, 12%, 13%, 14%, 15%, 16%, 17% or 18%. In various embodiments, the reference level may be a cyto-membranous 2+ or 3+ % TC of 8%, 9%, 10%, 11%, 12%, 13%, 14%, 15%, or 16%. In various embodiments, the reference level may be a cyto-membranous 2+ or 3+ % TC of 8%, 9%, 10%, 11%, 12%, 13%, 14%, or 15%. In various embodiments, the reference level may be a cyto-membranous 2+ or 3+ % TC of 8%, 9%, 10%, 11%, 12%, 13%, or 14%. In various embodiments, the reference level may be a cyto-membranous 2+ or 3+ % TC of 9%, 10%, 11%, 12%, or 13%. In various embodiments, the reference level may be a cyto-membranous 2+ or 3+ % TC of 9%, 10%, 11%, or 12%. In various embodiments, the reference level may be a cyto-membranous 2+ or 3+ % TC of 9%, 10%, or 11%.

In some embodiments, the CD155 expression level for a sample is characterized by a membranous 2+ or 3+ % TC. In some embodiments, the reference level may be ≤30%, ≤25%, ≤20%, ≤15%, ≤10%, or ≤5% above or below the median (e.g., 10% in gastric-intestinal cancers). In various embodiments, the reference level may be a membranous 2⁺ or 3+ % TC of 8%, 9%, 10%, 11%, 12%, 13%, 14%, or 15%. In various embodiments, the reference level may be a membranous 2+ or 3+ % TC of 8%, 9%, 10%, 11%, 12%, 13%, or 14%. In various embodiments, the reference level may be a membranous 2+ or 3+ % TC of 9%, 10%, 11%, 12%, or 13%. In various embodiments, the reference level may be a membranous 2+ or 3+ % TC of 9%, 10%, 11%, or 12%. In various embodiments, the reference level may be a membranous 2+ or 3+ % TC of 9%, 10%, or 11%.

In some embodiments, the CD155 expression level for a sample is characterized by a cyto-membranous H-score, in which the positive staining in the membrane and cytoplasm are combined. In some embodiments, the reference level may be ≤30%, ≤25%, ≤20%, ≤15%, ≤10%, or ≤5% above or below the median (e.g., 105 in gastric-intestinal cancers). In various embodiments, the reference level may be a cyto-membranous H-score of 100, 101, 102, 103, 104, 105, 106, 107, 108, 109, 110, 111, 112, 113, 114 or 115. In various embodiments, the reference level may be a cyto-membranous H-score of 100, 101, 102, 103, 104, 105, 106, 107, 108, 109, 110, 111, 112, or 113. In various embodiments, the reference level may be a cyto-membranous H-score of 101, 102, 103, 104, 105, 106, 107, 108, 109, 110, 111, or 112. In various embodiments, the reference level may be a cyto-membranous H-score of 102, 103, 104, 105, 106, 107, 108, 109, or 110. In various embodiments, the reference level may be a cyto-membranous H-score of 103, 104, 105, 106, 107, or 108. In various embodiments, the reference level may be a cyto-membranous H-score of 104, 105, or 106.

In some embodiments, the CD155 expression level for a sample is characterized by a membranous H-score. In some embodiments, the reference level may be ≤30%, ≤25%, ≤20%, ≤15%, ≤10%, or ≤5% above or below the median (e.g., 39 in gastric-intestinal cancers). In various embodiments, the reference level may be a membranous H-score of 22, 24, 26, 28, 30, 32, 34, 36, 38, 40, 42, 44, 46, 48 or 50. In various embodiments, the reference level may be a membranous H-score of 24, 26, 28, 30, 32, 34, 36, 38, 39, 40, 42, 44, 46, or 48. In various embodiments, the reference level may be a membranous H-score of 26, 28, 30, 32, 34, 36, 38, 39, 40, 42, 44, or 46. In various embodiments, the reference level may be a membranous H-score of 28, 30, 32, 34, 36, 38, 39, 40, 42, or 44. In various embodiments, the reference level may be a membranous H-score of 32, 34, 36, 38, 39, 40, 42, or 44. In various embodiments, the reference level may be a membranous H-score of 34, 36, 38, 39, 40, or 42. In various embodiments, the reference level may be a membranous H-score of, 38, 39, or 40.

### Methods of Prognosis

In one aspect, the present disclosure provides a method for determining a prognosis of a patient having a disease. In some embodiments, the present disclosure provides a method for determining a prognosis of a patient having a particular disease, wherein the patient has received a therapy for the disease. The biomarkers of the below embodiments are further described elsewhere herein, and those disclosures are incorporated by reference into this section. Suitable samples are also described elsewhere herein and incorporated by reference into this section.

The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition (e.g., a biomarker), when compared to those individuals not exhibiting the condition. A prognosis can be expressed as the amount of time a patient can be expected to survive. Alternatively, a prognosis can refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively, prognosis can be expressed as the number of years, on average that a patient can expect to survive as a result of a condition or disease. The prognosis of a patient can be considered as an expression of relativism, with many factors effecting the ultimate outcome. For example, for patients with certain conditions, prognosis can be appropriately expressed as the likelihood that a condition can be treatable or curable, or the likelihood that a disease will go into remission, whereas for patients with more severe conditions prognosis can be more appropriately expressed as likelihood of survival for a specified period of time.

The term “poor prognosis” as used herein, in the context of a patient having cancer, refers to an increased likelihood that the patient will have a reduced duration of remission, reduced duration of progression free survival, reduced survival rate, or reduced survival duration, relative to that of a patient who does not share the same biomarker expression profile, as described herein.

The term “favorable prognosis” as used herein, in the context of a patient having cancer, refers to an increased likelihood that the patient will have an increased duration of remission, increased duration of progression free survival, increased survival rate, or increased survival duration, relative to that of a patient who does not share the same biomarker expression profile, as described herein.

In one aspect, the present disclosure provides biomarkers useful for determining a prognosis for a patient, such as a patient identified as having cancer. The biomarkers are useful for determining a prognosis for a patient who has received or will receive a therapy for cancer, such as a therapy comprising an immune checkpoint inhibitor. In some embodiments, the biomarker is selected from PD-L1 (PD-L1 expression level), CD155 (CD155 expression level), CD226 (CD226 expression level), an adenosine pathway biomarker, CD73 (CD73 expression level), and any combination thereof. In some embodiments, the adenosine pathway biomarker is CD73 (CD73 expression level). A prognosis for a patient who has received or will receive a therapy comprising an immune checkpoint inhibitor may be determined when a sample obtained from the patient comprises: (a) a PD-L1 expression level greater than or equal to a PD-L1 reference level or lower than a PD-L1 reference level; (b) a CD155 expression level greater than or equal to a CD155 reference level or lower than a CD155 reference level; (c) a CD226 expression level greater than or equal to a CD226 reference level or lower than a CD226 reference level; (d) an adenosine pathway biomarker expression level greater than or equal to an adenosine pathway biomarker reference level or lower than an adenosine pathway biomarker reference level; (e) a CD73 expression level greater than or equal to a CD73 reference level or lower than a CD73 reference level or (f) any combination of (a) to (e).

In some embodiments, a patient who has received or will receive a therapy comprising an immune checkpoint inhibitor may be determined to have a favorable prognosis when a sample obtained from the patient comprises (a) a PD-L1 expression level greater than or equal to a PD-L1 reference level; (b) a CD155 expression level lower than a CD155 reference level; (c) a CD226 expression level greater than or equal to a CD226 reference level; (d) an adenosine pathway biomarker expression level greater than or equal to an adenosine pathway biomarker reference level; (e) a CD73 expression level greater than or equal to a CD73 reference level, or (f) any combination of (a) to (e). In some embodiments, a patient who has received or will receive a therapy comprising an immune checkpoint inhibitor may be determined to have a poor prognosis when a sample obtained from the patient comprises (a) a PD-L1 expression level lower than a PD-L1 reference level; (b) a CD155 expression level greater than or equal to a CD155 reference level; (c) a CD226 expression level lower than a CD226 reference level; (d) an adenosine pathway biomarker expression level lower than an adenosine pathway biomarker reference level; (e) a CD73 expression level lower than a reference level, or (f) any combination of (a) to (e). In some embodiments of the above, the therapy that a patient has received, or will receive, comprises or consists of a single immune checkpoint inhibitor. In some embodiments, the therapy is a monotherapy (e.g., a single immune checkpoint inhibitor). In some embodiments, the therapy is a combination therapy comprising a single immune checkpoint inhibitor and one or more than one additional therapy that is not an immune checkpoint inhibitor (e.g., chemotherapy (optionally a platinum-containing chemotherapy), a VEGF or VEGFR inhibitor, an ATP-adenosine axis-targeting agent, etc.). In certain embodiments, the single immune checkpoint inhibitor is an PD-L1 antagonist or an PD-1 antagonist (designated herein as “PD-(L)1 antagonist”), optionally an anti-PD-L1 antibody or an anti-PD-1 antibody (designated herein as “anti-PD-(L)1 antibody”).

In some embodiments, a patient who has received or will receive a therapy comprising an immune checkpoint inhibitor may be determined to have a favorable prognosis when a sample obtained from the patient comprises (a) a PD-L1 expression level greater than or equal to a PD-L1 reference level; (b) a CD155 expression level lower than a CD155 reference level; (c) a CD226 expression level greater than or equal to a CD226 reference level; (d) an adenosine pathway biomarker expression level lower than an adenosine pathway biomarker reference level; (e) a CD73 expression level lower than a CD73 reference level, or (f) any combination of (a) to (e). In some embodiments, a patient who has received or will receive a therapy comprising an immune checkpoint inhibitor (e.g., a PD-(L)1 antagonist) may be determined to have a poor prognosis when a sample obtained from the patient comprises (a) a PD-L1 expression level lower than a PD-L1 reference level; (b) a CD155 expression level greater than or equal to a CD155 reference level; (c) a CD226 expression level lower than a CD226 reference level; (d) an adenosine pathway biomarker expression level greater than or equal to an adenosine pathway biomarker reference level; (e) a CD73 expression level greater than or equal to a CD73 reference level, or (f) any combination of (a) to (e). In some embodiments of the above, the therapy that a patient has received, or will receive, is a combination therapy comprising or consisting of a first and a second immune checkpoint inhibitor. In embodiments comprising a first and a second immune checkpoint inhibitor, the therapy may further comprise one or more than one additional therapy (e.g., chemotherapy, a VEGF or VEGFR inhibitor, an ATP-adenosine axis-targeting agent, etc.). In certain embodiments, either the first or the second checkpoint inhibitor is a PD-L1 antagonist or a PD-1 antagonist (designated herein as “PD-(L)1 antagonist”), optionally an anti-PD-L1 antibody or an anti-PD-1 antibody (designated herein as “anti-PD-(L)1 antibody”). In certain embodiments, either the first or the second checkpoint inhibitor is a TIGIT antagonist, optionally an anti-TIGIT antibody. In some embodiments, the first immune checkpoint inhibitor is a PD-(L)1 antagonist and the second immune checkpoint inhibitor is a TIGIT antagonist.

In the above embodiments, the sample can be a sample comprising tumor cells, immune cells (e.g., lymphocytes, monocytes, myeloid cells, dendritic cells, plasma cells, etc.), other cell types of the tumor microenvironment, or any combination thereof.

In embodiments comprising a combination therapy, the combination therapy may comprise at least two therapies (i.e., 2, 3, 4, 5, or more therapies). In some embodiments, the combination therapy comprises at least three therapies (i.e., 3, 4, 5, 6, or more therapies). In some embodiments, the combination can include an anti-PD-1 antibody. In some embodiments, the combination can include an anti-PD-L1 antibody. In some embodiments, the combination can include an anti-TIGIT antibody. In some embodiments, the combination can include an anti-PD-1 antibody and an anti-TIGIT antibody. In some embodiments, the combination can include an anti-PD-L1 antibody and an anti-TIGIT antibody. In embodiments comprising an anti-TIGIT antibody, the anti-TIGIT antibody may be an Fc-silent anti-TIGIT antibody.

In some embodiments, the combination can include an ATP-adenosine axis-targeting agent. In some embodiments, the combination can include an anti-PD-1 antibody, an anti-TIGIT antibody, and an ATP-adenosine axis-targeting agent. In some embodiments, the combination can include an anti-PD-L1 antibody, an anti-TIGIT antibody, and an ATP-adenosine axis-targeting agent. In some embodiments, the combination can include an anti-PD-(L)1 antibody, an anti-TIGIT antibody, and an ATP-adenosine axis-targeting agent. In some embodiments, the ATP-adenosine axis-targeting agent is an adenosine receptor antagonist. In some embodiments, the ATP-adenosine axis-targeting agent is an A₂ₐR and/or A_{2b}R antagonist, a CD73 inhibitor, or a CD39 inhibitor. In some embodiments, the ATP-adenosine axis-targeting agent is an A₂ₐR and/or A_{2b}R antagonist.

In some embodiments, a combination therapy comprises chemotherapy, such as a chemotherapy agent or a chemotherapy regimen described herein. In some embodiments, the chemotherapy comprises a platinum-containing agent.

In some embodiments, a patient for which a prognosis is determined, as described herein, has cancer comprising a solid tumor. In some embodiments, the patient has a solid tumor selected from the group consisting of ovarian cancer, endometrial cancer, breast cancer, lung cancer, colon cancer, prostate cancer, cervical cancer, biliary cancer, pancreatic cancer, gastric cancer, esophageal cancer, liver cancer, kidney cancer, head-and-neck tumors, mesothelioma, melanoma, sarcomas, central nervous system (CNS) hemangioblastomas, and brain tumors. In some embodiments, the patient has a cancer selected from the group consisting of gastrointestinal cancer, genitourinary cancer, gynecological cancer, and lung cancer. In some embodiments, the patient has lung cancer, such as, without limitation, squamous cell lung cancer or non-squamous cell lung cancer (NSCLC). In some embodiments, the patient has upper gastrointestinal (GI) cancer, such as, without limitation, gastric cancer, gastroesophageal junction (GEJ) cancer, or esophageal adenocarcinoma (EAC). The cancer may be a locally advanced unresectable cancer. The cancer may be recurrent or metastatic cancer.

In some embodiments, a biomarker expression level in a sample is determined by immunohistochemistry (IHC). Expression level can be measured or determined based on IHC staining intensity and/or percentage of staining-positive cells (e.g., tumor cells and/or immune cells). In some embodiments, a reference level is determined by IHC. In some embodiments, the IHC staining intensity and/or the percentage of staining-positive cells is determined based on cytoplasmic staining, membrane staining, or both cytoplasmic and membrane staining. Methods of using IHC to determine the level of a biomarker in a sample are known in the art and further described herein.

In some embodiments, a reference level is a median expression level, or an amount about 30%, about 25%, about 20%, about 15%, about 10%, or about 5% above or below a median expression level, derived from samples obtained from a group of patients known to have the same disease. For example, in some embodiments, a PD-L1 reference level can be an amount about 30%, about 25%, about 20%, about 15%, about 10%, or about 5% above or below a median PD-L1 expression level derived from samples obtained from a group of patients known to have the same disease. In some embodiments, a CD155 reference level can be an amount about 30%, about 25%, about 20%, about 15%, about 10%, or about 5% above or below a median CD155 expression level derived from samples obtained from a group of patients known to have the same disease. In some embodiments, a CD226 reference level can be an amount about 30%, about 25%, about 20%, about 15%, about 10%, or about 5% above or below a median CD226 expression level derived from samples obtained from a group of patients known to have the same disease. In some embodiments, an adenosine pathway biomarker reference level can be an amount about 30%, about 25%, about 20%, about 15%, about 10%, or about 5% above or below a median adenosine pathway biomarker expression level derived from samples obtained from a group patients known to have the same disease. In some embodiments, a CD73 reference level can be an amount about 30%, about 25%, about 20%, about 15%, about 10%, or about 5% above or below a median CD73 expression level derived from samples obtained from a group of patients known to have the same disease.

In some embodiments, a patient is determined to have a favorable prognosis when, in a sample obtained from the patient: (a) a PD-L1 expression level is greater than or equal to a median PD-L1 expression level or greater than or equal to an amount ≤30%, ≤25%, ≤20%, ≤15%, ≤10%, or ≤5% above or below a median PD-L1 expression level derived from samples obtained from a group of patients known to have the same disease; (b) a CD155 expression level is lower than a median CD155 expression level or lower than an amount ≤30%, ≤25%, ≤20%, ≤15%, ≤10%, or ≤5% above or below a median CD155 expression level derived from samples obtained from a group of patients known to have the same disease; (c) a CD226 expression level is greater than or equal to a median CD226 expression level or greater than or equal to an amount ≤30%, ≤25%, ≤20%, ≤15%, ≤10%, or ≤5% above or below a median CD226 expression level derived from samples obtained from a group of patients known to have the same disease; (d) an adenosine pathway biomarker expression level is greater than or equal to a median adenosine pathway biomarker expression level or greater than or equal to an amount ≤30%, ≤25%, ≤20%, ≤15%, ≤10%, or ≤5% above or below a median adenosine pathway biomarker expression level derived from samples obtained from a group of patients known to have the same disease; (e) a CD73 expression level is greater than or equal to a median CD73 expression level or greater than or equal to an amount ≤30%, ≤25%, ≤20%, ≤15%, ≤10%, or ≤5% above or below a median CD73 expression level derived from samples obtained from a group of patients known to have the same disease; or (f) any combination of (a) to (e). In some embodiments of the above, the therapy that a patient has received, or will receive, comprises or consists of a single immune checkpoint inhibitor. In some embodiments, the therapy is a monotherapy (e.g., a single immune checkpoint inhibitor). In some embodiments, the therapy is a combination therapy comprising a single immune checkpoint inhibitor and one or more than one additional therapy that is not an immune checkpoint inhibitor (e.g., chemotherapy, a VEGF or VEGFR inhibitor, an ATP-adenosine axis-targeting agent, etc.) In certain embodiments, the single immune checkpoint inhibitor is an PD-L1 antagonist or an PD-1 antagonist (designated herein as “PD-(L)1 antagonist”), optionally an anti-PD-L1 antibody or an anti-PD-1 antibody (designated herein as “anti-PD-(L)1 antibody”).

In some embodiments, a patient is determined to have a favorable prognosis when, in a sample obtained from the patient: (a) a PD-L1 expression level is greater than or equal to a median PD-L1 expression level or greater than or equal to an amount ≤30%, ≤25%, ≤20%, ≤15%, ≤10%, or ≤5% above or below a median PD-L1 expression level derived from samples obtained from a group of patients known to have the same disease; (b) a CD155 expression level is lower than a median CD155 expression level or lower than an amount ≤30%, ≤25%, ≤20%, ≤15%, ≤10%, or ≤5% above or below a median CD155 expression level derived from samples obtained from a group of patients known to have the same disease; (c) a CD226 expression level is greater than or equal to a median CD226 expression level or greater than or equal to an amount ≤30%, ≤25%, ≤20%, ≤15%, ≤10%, or ≤5% above or below a median CD226 expression level derived from samples obtained from a group of patients known to have the same disease; (d) an adenosine pathway biomarker expression level is lower than a median adenosine pathway biomarker expression level or lower than an amount ≤30%, ≤25%, ≤20%, ≤15%, ≤10%, or ≤5% above or below a median adenosine pathway biomarker expression level derived from samples obtained from a group of patients known to have the same disease; (e) a CD73 expression level is lower than a median CD73 expression level or lower than an amount ≤30%, ≤25%, ≤20%, ≤15%, ≤10%, or ≤5% above or below a median CD73 expression level derived from samples obtained from a group of patients known to have the same disease; or (f) any combination of (a) to (e). In some embodiments of the above, the therapy that a patient has received, or will receive, is a combination therapy comprising or consisting of a first and a second immune checkpoint inhibitor. In embodiments comprising a first and a second immune checkpoint inhibitor, the therapy may further comprise one or more than one additional therapy (e.g., chemotherapy, a VEGF or VEGFR inhibitor, an ATP-adenosine axis-targeting agent, etc.). In certain embodiments, either the first or the second checkpoint inhibitor is a PD-L1 antagonist or a PD-1 antagonist (designated herein as “PD-(L)1 antagonist”), optionally an anti-PD-L1 antibody or an anti-PD-1 antibody (designated herein as “anti-PD-(L)1 antibody”). In certain embodiments, either the first or the second checkpoint inhibitor is a TIGIT antagonist, optionally an anti-TIGIT antibody. In some embodiments, the first immune checkpoint inhibitor is a PD-(L)1 antagonist and the second immune checkpoint inhibitor is a TIGIT antagonist.

In some embodiments, a patient is determined to have a poor prognosis when, in a sample obtained from the patient: (a) a PD-L1 expression level is lower than a median PD-L1 expression level or lower than an amount ≤30%, ≤25%, ≤20%, ≤15%, ≤10%, or ≤5% above or below a median PD-L1 expression level derived from samples obtained from a group of patients known to have the same disease; (b) a CD155 expression level is greater than or equal to a median CD155 expression level or greater than or equal to an amount ≤30%, ≤25%, ≤20%, ≤15%, ≤10%, or ≤5% above or below a median CD155 expression level derived from samples obtained from a group of patients known to have the same disease; (c) a CD226 expression level is lower than a median CD226 expression level or lower than an amount ≤30%, ≤25%, ≤20%, ≤15%, ≤10%, or ≤5% above or below a median CD226 expression level derived from samples obtained from a group of patients known to have the same disease; (d) an adenosine pathway biomarker expression level is lower than a median adenosine pathway biomarker expression level or lower than an amount ≤30%, ≤25%, ≤20%, ≤15%, ≤10%, or ≤5% above or below a median adenosine pathway biomarker expression level derived from samples obtained from a group of patients known to have the same disease; (e) a CD73 expression level is lower than a median CD73 expression level or lower than an amount ≤30%, ≤25%, ≤20%, ≤15%, ≤10%, or ≤5% above or below a median CD73 expression level derived from samples obtained from a group of patients known to have the same disease; or (f) any combination of (a) to (e). In some embodiments of the above, the therapy that a patient has received, or will receive, comprises or consists of a single immune checkpoint inhibitor. In some embodiments, the therapy is a monotherapy (e.g., a single immune checkpoint inhibitor). In some embodiments, the therapy is a combination therapy comprising a single immune checkpoint inhibitor and one or more than one additional therapy that is not an immune checkpoint inhibitor (e.g., chemotherapy, a VEGF or VEGFR inhibitor, an ATP-adenosine axis-targeting agent, etc.) In certain embodiments, the single immune checkpoint inhibitor is an PD-L1 antagonist or an PD-1 antagonist (designated herein as “PD-(L)1 antagonist”), optionally an anti-PD-L1 antibody or an anti-PD-1 antibody (designated herein as “anti-PD-(L)1 antibody”).

In some embodiments, a patient is determined to have a poor prognosis when, in a sample obtained from the patient: (a) a PD-L1 expression level is lower than a median PD-L1 expression level or lower than an amount ≤30%, ≤25%, ≤20%, ≤15%, ≤10%, or ≤5% above or below a median PD-L1 expression level derived from samples obtained from a group of patients known to have the same disease; (b) a CD155 expression level is greater than or equal to a median CD155 expression level or greater than or equal to an amount ≤30%, ≤25%, ≤20%, ≤15%, ≤10%, or ≤5% above or below a median CD155 expression level derived from samples obtained from a group of patients known to have the same disease; (c) a CD226 expression level is lower than a median CD226 expression level or lower than an amount ≤30%, ≤25%, ≤20%, ≤15%, ≤10%, or ≤5% above or below a median CD226 expression level derived from samples obtained from a group of patients, known to have the same disease; (d) an adenosine pathway biomarker expression level is greater than or equal to a median adenosine pathway biomarker expression level or greater than or equal to an amount ≤30%, ≤25%, ≤20%, ≤15%, ≤10%, or ≤5% above or below a median adenosine pathway biomarker expression level derived from samples obtained from a group of patients known to have the same disease (e) a CD73 expression level is greater than or equal to a median CD73 expression level or greater than or equal to an amount ≤30%, ≤25%, ≤20%, ≤15%, ≤10%, or ≤5% above or below a median CD73 expression level derived from samples obtained from a group of patients known to have the same disease; or (f) any combination of (a) to (e). In some embodiments of the above, the therapy that a patient has received, or will receive, is a combination therapy comprising or consisting of a first and a second immune checkpoint inhibitor. In embodiments comprising a first and a second immune checkpoint inhibitor, the therapy may further comprise one or more than one additional therapy (e.g., chemotherapy, a VEGF or VEGFR inhibitor, an ATP-adenosine axis-targeting agent, etc.). In certain embodiments, either the first or the second checkpoint inhibitor is a PD-L1 antagonist or a PD-1 antagonist (designated herein as “PD-(L)1 antagonist”), optionally an anti-PD-L1 antibody or an anti-PD-1 antibody (designated herein as “anti-PD-(L)1 antibody”). In certain embodiments, either the first or the second checkpoint inhibitor is a TIGIT antagonist, optionally an anti-TIGIT antibody. In some embodiments, the first immune checkpoint inhibitor is a PD-(L)1 antagonist and the second immune checkpoint inhibitor is a TIGIT antagonist.

In some embodiments, expression levels may be expressed in numerous ways, including but not limited to biomarker-positive tumor cell fraction (“% TC”), biomarker-positive tumor cell fraction staining at medium or high intensity (“2+ or 3+ % TC”), biomarker-positive immune cell fraction (“% IC”), characterization by a tumor H-score, and characterization of expression relative to tumor area (e.g., the number of biomarker-positive tumor cells showing positive staining at any intensity and/or the number of biomarker-positive tumor-associated immune cells showing positive staining at any intensity relative to tumor area), as described elsewhere herein.

### Oncology and Oncology-Related Disorders

In one or more embodiments of this disclosure, the biomarkers described herein are useful in the treatment or prognosis of a disease, e.g., cancer. In certain embodiments, the cancer may be early stage cancer, e.g., Stage I or Stage II. In other embodiments, the cancer may be locally advanced and/or unresectable, metastatic, or at risk of becoming metastatic. Alternatively, or in addition, the cancer may be recurrent or no longer responding to a treatment, such as a standard of care treatment known to one of skill in the art. Exemplary types of cancer contemplated by this disclosure include cancer of the genitourinary tract (e.g., bladder, kidney, renal cell, penile, prostate, testicular, etc.), uterus, cervix, ovary, breast, gastrointestinal tract (e.g., esophagus, oropharynx, stomach, small or large intestines, colon, or rectum), bone, bone marrow, skin (e.g., melanoma), head and neck, liver, gall bladder, bile ducts, heart, lung, pancreas, salivary gland, adrenal gland, thyroid, brain (e.g., gliomas), ganglia, central nervous system (CNS), peripheral nervous system (PNS), the hematopoietic system (i.e., hematological malignancies), and the immune system (e.g., spleen or thymus).

In some embodiments, the biomarkers according to this disclosure are useful in the treatment or prognosis of hematological malignancies. Exemplary types of cancer affecting the hematopoietic system include leukemias, lymphomas and myelomas, including acute myeloid leukemia, adult T-cell leukemia, T-cell large granular lymphocyte leukemia, acute lymphoblastic leukemia, chronic lymphocytic leukemia, chronic myelogenous leukemia, acute monocytic leukemia, Hodgkin's and Non-Hodgkin's lymphoma, Diffuse large B Cell lymphoma, and multiple myeloma.

In another embodiment, the biomarkers according to this disclosure are useful in the treatment or prognosis of solid tumors. The solid tumor may be, for example, ovarian cancer, endometrial cancer, breast cancer, lung cancer (small cell or non-small cell), colon cancer, prostate cancer, cervical cancer, biliary cancer, pancreatic cancer, gastric cancer, esophageal cancer, liver cancer (e.g., hepatocellular carcinoma), kidney cancer (e.g., renal cell carcinoma), head-and-neck tumors, mesothelioma, melanoma, sarcomas, central nervous system (CNS) hemangioblastomas, and brain tumors (e.g., gliomas, such as astrocytoma, oligodendroglioma and glioblastomas).

In some embodiments, the biomarkers according to this disclosure are useful in the treatment or prognosis of gastrointestinal cancer, genitourinary cancer, gynecological cancer, lung cancer, or a combination thereof.

In some embodiments, the biomarkers according to this disclosure are useful in the treatment or prognosis of gastrointestinal (GI) cancer. In some embodiments, the GI cancer is colorectal cancer, pancreatic cancer, or liver cancer. In some embodiments, the GI cancer is an upper GI cancer, such as esophageal or gastric cancer. In further embodiments, the upper GI cancer is an adenocarcinoma, a squamous cell carcinoma, or any combination thereof. In still further embodiments, the upper GI cancer is esophageal adenocarcinoma (EAC), esophageal squamous cell carcinoma (ESCC), gastroesophageal junction adenocarcinoma (GEJ), gastric adenocarcinoma (also referred to herein as “gastric cancer”) or any combination thereof.

In some embodiments, a subject in need of the treatments or methods described herein may be a subject with gastrointestinal cancer, optionally an (i) upper GI cancer, (ii) a GI cancer selected from the group consisting of GA, GEJ, ESCC, EAC, and any combination thereof, (iii) a GI cancer selected from the group consisting of GA, GEJ, EAC, and any combination thereof; and in certain embodiments a patient with early stage disease (Stage I or Stage II), and in other embodiments a patient with locally advanced unresectable or metastatic disease. The subject may or may not have had treatment with a prior systemic treatment and may be an immune checkpoint inhibitor (CPI) naïve or experienced. In addition, the subject may or may not have been screened for biomarkers such as microsatellite instability (MSI) by PCR or NGS, mismatch repair (MMR) testing by IHC, and/or HER2 expression by IHC or HER2 copy number by ISH or FISH. In some embodiments, the cancer may be MSI-Stable or MSI-Low, as determined by a clinically validated or an FDA-approved test. In some embodiments, the cancer may be MSI-High, as determined by a clinically validated or an FDA-approved test. In some embodiments, the cancer may be a HER-2 positive, as determined by a clinically validated or an FDA-approved test. In some embodiments, the cancer may be HER-2 negative, as determined by a clinically validated or an FDA-approved test. In some embodiments, the cancer may be PD-L1 positive, e.g., TAP (tumor area positivity) ≥1%, ≥5%, ≥10%, 1% to <5%, 5% to <10%, or >10%, measured by the Ventana SP263 IHC Assay, or an equivalent value measured by another clinically validated PD-L1 IHC assay. In some embodiments, the cancer may be PD-L1 negative, e.g., TAP <1%.

In some embodiments, the biomarkers according to this disclosure are useful in the treatment or prognosis of pancreatic cancer. In further embodiments, the pancreatic cancer is pancreatic neuroendocrine tumor or pancreatic adenocarcinoma.

In some embodiments, the biomarkers according to this disclosure are useful in the treatment or prognosis of liver cancer. In further embodiments, the liver cancer is hepatocellular carcinoma. In other embodiments, the liver cancer is liver metastases.

In some embodiments, the biomarkers according to this disclosure are useful in the treatment or prognosis of genitourinary cancer. In some embodiments, the genitourinary cancer is bladder cancer, kidney cancer or prostate cancer.

In some embodiments, the biomarkers according to this disclosure are useful in the treatment or prognosis of kidney cancer. In further embodiments, the kidney cancer is renal cell carcinoma. In still further embodiments, the renal cell carcinoma is clear cell renal carcinoma.

In some embodiments, the according to this disclosure are useful in the treatment or prognosis of gynecological cancer. In some embodiments, the gynecological cancer is breast cancer, endometrial cancer, or ovarian cancer. In some embodiments, the gynecological cancer is hormone receptor positive (e.g., ERα-positive cancer, PR-positive cancer, ERα-positive and PR-positive cancer), HER2 positive cancer, HER2 over-expressing cancer, or any combination thereof. In still further embodiments, the cancer is triple negative cancer (e.g., ER, PR and HER2 negative).

In some embodiments, the biomarkers according to this disclosure are useful in the treatment or prognosis of lung cancer. In further embodiments, the lung cancer is mesothelioma, small cell lung cancer (SCLC) or non-small cell lung cancer (NSCLC). In still further embodiments, the lung cancer is NSCLC, optionally lung squamous cell carcinoma or lung adenocarcinoma.

In some embodiments, a subject in need of the treatments or methods described herein may be a human subject with NSCLC (squamous or non-squamous disease), and in certain embodiments a patient with early-stage disease (Stage I or II) or resectable Stage II or Stage III NSCLC, and in other embodiments a patient with unresectable locally advanced disease (Stage IIIA, IIIB, IIIC) or metastatic disease (Stage IV). In some embodiments, the NSCLC may be PD-L1 high, corresponding to TPS ≥50% or TC ≥50% as measured by a clinically validated PD-L1 IHC assay or FDA-approved test, such as PharmDx 22C3 IHC assay, 28-8 pharmDx (Dako), Ventana SP263 IHC assay, or Ventana SP142 IHC assay. In some embodiments, the NSCLC may be PD-L1 positive, corresponding to TPS ≥1% and <50% or TC ≥1% and <50% as measured by a clinically validated PD-L1 IHC assay or FDA-approved test, for example, about 1%, about 5%, about 10%, about 15%, about 20%, about 25%, about 30%, about 35%, about 40%, about 45%, about 46%, about 47%, about 48% about 49%, or ranges thereof such as about 1-10%, about 10%-20%, about 20-30%, about 30-40%, about 40-49%, about 1-49%, about 1-25%, or about 25-49%. In some embodiments, the NSCLC may be PD-L1 expressing, corresponding to TPS >10%, ≥10%, ≥15%, ≥20%, ≥25%, ≥30%, ≥35%, ≥40%, or >45%, or TC ≥10%, ≥10%, ≥15%, ≥20%, ≥25%, ≥30%, ≥35%, ≥40%, or ≥45%, as measured by a clinically validated PD-L1 IHC assay or FDA-approved test. In some embodiments, the cancer may be tumor mutational burden-high (TMB-H; ≥10 mutations/megabase (mut/Mb), as determined by an FDA-approved test). The cancer may or may not have a genomic mutation for which a targeted therapy has received marketing approval by a regulatory authority, non-limiting examples of genes with such mutation include ALK fusion oncogene, EGFR, ROS, BRAF, and NTRK. In some embodiments, the cancer may be EGFR and/or ALK wildtype or may have a mutation in EGFR or ALK but the mutation is not an actionable mutation (i.e., there is not a targeted therapy available for treatment of a cancer with that mutation).

In some embodiments, the biomarkers according to this disclosure are useful in the treatment or prognosis of a neuroendocrine tumor. In further embodiments, the neuroendocrine tumor is pancreatic neuroendocrine tumor, pheochromocytoma, paraganglioma, or a tumor of the adrenal gland.

In some embodiments, the biomarkers according to this disclosure are useful in the treatment or prognosis of brain cancer. In further embodiments, the brain cancer is a glioma. In still further embodiments, the glioma is an astrocytoma, an oligodendroglioma, or a glioblastoma.

In some embodiments, the biomarkers according to this disclosure are useful in the treatment or prognosis of head and neck cancer. In further embodiments, the head and neck cancer is head and neck squamous cell carcinoma.

In the aforementioned embodiments, the methods of the present disclosure may be practiced in neoadjuvant setting, an adjuvant setting, or neoadjuvant and adjuvant setting. Alternatively or in addition, the methods described herein may be indicated as a first line treatment, optionally in the treatment of locally advanced, unresectable, or metastatic cancer. In some embodiments, the methods described herein may be indicated as a second line, third line, or greater line of treatment, optionally in the treatment of locally advanced, unresectable, or metastatic cancer. When indicated as a second line or greater treatment, in some embodiments an earlier line of therapy included an immune checkpoint inhibitor (e.g., a PD-(L)1 antagonist), a targeted therapy (e.g., a therapy directed to an actionable mutation), chemotherapy (e.g., chemotherapy comprising a platinum agent), or any combination thereof. With respect to prior treatment with an immune checkpoint inhibitor, subject may be described as “immune checkpoint inhibitor experienced” if previously treated with an immune checkpoint inhibitor or “immune checkpoint inhibitor naïve” if not previously treated with a checkpoint inhibitor for the cancer in question. In some embodiments, a subject in need of treatment is immune checkpoint inhibitor experienced. In some embodiments, a subject in need of treatment is immune checkpoint inhibitor naive. In some embodiments, a subject in need of treatment is previously untreated with chemotherapy. In some embodiments, a subject in need of treatment is previously treated with chemotherapy, optionally a chemotherapy comprising a platinum agent.

The present disclosure also provides methods of treating or preventing other cancer-related diseases, disorders or conditions. The use of the term(s) cancer-related diseases, disorders and conditions is meant to refer broadly to conditions that are associated, directly or indirectly, with cancer and non-cancerous proliferative disease, and includes, e.g., angiogenesis, precancerous conditions such as dysplasia, and non-cancerous proliferative diseases disorders or conditions, such as benign proliferative breast disease and papillomas. For clarity, the term(s) cancer-related disease, disorder and condition do not include cancer per se.

In general, the disclosed methods for treating cancer, or a cancer-related disease, disorder or condition, in a subject in need thereof comprise administering to the subject a therapy described herein. In some embodiments, the present disclosure provides methods for treating cancer, or a cancer-related disease, disorder or condition with a therapy described herein and at least one additional therapy, examples of which are set forth elsewhere herein.

### EXAMPLES

The following examples are put forth so as to provide those of ordinary skill in the art with a complete disclosure and description of how to make and use the present disclosure, and are not intended to limit the scope of what the inventors regard as their invention. While certain embodiments have been illustrated and described, it should be understood that changes and modifications can be made therein in accordance with ordinary skill in the art without departing from the technology in its broader aspects as defined in the following claims.

### Example 1

CD226, CD155 and CD73 expression levels were evaluated in formalin-fixed, paraffin embedded (FFPE) human tissue by immunohistochemistry using validated assays. Briefly, serial 4-5 micron sections of FFPE blocks were mounted on positive charged glass slides and stained with hematoxylin and eosin (H&E) or for protein expression using DAB chromagen following staining with a primary antibody (CD226: rabbit clone 102 (Sino Biological); CD155: rabbit clone D3G7H; CD73: rabbit clone D7F9A (Cell Signaling)). Stained slides were evaluated by a pathologist. H&E staining was used to measure percentage of tumor and necrosis and to annotate areas of tumor to guide nucleic acid extraction. CD155 and CD73 staining was measured on a semi-quantitative scale: 0=negative, 1+=weak intensity, 2+=moderate intensity, and 3+=strong intensity. Staining intensity and percentage of cells staining in cytoplasm and membrane was recorded separately.

The localization of CD155 and CD73 staining within tumor cells can be cytoplasmic and membranous. For calculations of CD155 and CD73 expression in tumor cells, positive staining in the membrane and cytoplasm were combined, unless indicated otherwise. A tumor sample was considered positive if at least 1% of tumor cells demonstrated positive expression. Protein expression level in tumor cells was characterized in a number of ways, including % TC, number of positive tumor cells relative to tumor area, tumor fractional intensities (e.g., percent of tumor cells staining at each intensity (e.g., 0, 1+, 2+, 3+) relative to total tumor cells), tumor H-score, etc.

Samples were also evaluated for the presence or absence of immune cells (e.g., lymphocytes, monocytes, myeloid cells, dendritic cells, plasma cells, etc.) and, when present, CD155 and CD73 positive immune cell staining intensity was recorded. The localization of CD155 and CD73 staining within immune cells can also be cytoplasmic and membrane, and positive staining at any location was included, unless indicated otherwise. Immune cell protein expression level was characterized in a number of ways including % IC, mononuclear inflammatory density score (MIDS), mononuclear inflammatory median intensity (MIMI), stromal percent positive score, stromal fractional intensities (0, 1+, 2+, 3+), stromal H-score, etc. MIDS is an estimate of the number of positive mononuclear inflammatory cells (MIC, staining at any intensity) and associated with tumor cells. MIC include lymphocytes, monocytes, and macrophages within the tumor nests and the adjacent supporting stroma. The density is estimated relative to the number of tumor cells using the following score: 0=0% (none), 1=<1% (present, but less than 1 MIC for every 100 tumor cells), 2=≥1% and <10% (at least 1 MIC for every 10 tumor cells but fewer MICs than tumor cells), 4=≥100% (at least as many MICs as tumor cells), NA (not applicable; no immune cells present), NE (not evaluable; only used when immune cells are present but not evaluable). MIMI estimates the mean intensity of positive MICs. Intensity is estimate per the usual scale (0, 1+, 2+, 3+). Stromal percent positive score includes fibroblasts and inflammatory cells within the stromal areas in the inter and peri-tumor. The denominator is all fibroblasts and inflammatory cells within the stromal areas.

Maximum staining intensity in other normal cell types was also evaluated and recorded (e.g., normal adjacent tissue (NAT), endothelia, smooth muscle, fibroblasts, stroma, inflammatory cells, and nerve).

CD226 cytoplasmic staining within immune cells was evaluated and scored visually by a pathologist. The staining intensity was scored per the usual scale (0, 1+, 2+, 3+) and subcellular location was noted (cytoplasmic, membranous, or both). The percentage of positive immune cells was recorded as well as an H-score and maximum signal intensity. Scores were also captured for the percentage of positive immune cells localized to the intra-tumoral (intra-epithelial) compartment and extra-tumoral (stromal/peri-tumoral) compartment. The threshold for CD226 positivity was 1% expression.

### Example 2

The potential treatment benefit of zimberelimab monotherapy vs. domvanalimab plus zimberelimab combination therapy vs. etrumadenant plus domvanalimab plus zimberelimab combination therapy was evaluated in 150 people as a first-line treatment for metastatic (Stage IV) squamous or non-squamous NSCLC. Participants were randomized 1:1:1 across the three study arms and treated until disease progression or loss of clinical benefit. Arm 1 received zimberelimab 360 mg IV Q3W (Arm 1=Z). Arm 2 received domvanalimab 15 mg/kg IV Q3W and zimberelimab 360 mg IV Q3W (Arm 2=DZ). Arm 3 received domvanalimab 15 mg/kg IV Q3W, zimberelimab 360 mg IV Q3W, and etrumadenant 150 mg PO QD (Arm 3=EDZ). Participants in the zimberelimab monotherapy arm (Arm 1) with confirmed progression had the option to cross over to the triplet arm (Arm 3).

Enrolled participants had an ECOG performance status of 0-1 and were EGFR/ALK wild-type. EGFR and ALK mutational testing was not required for participants with squamous histology. Baseline characteristics are shown in Table 1.

TABLE 1
Baseline characteristics of enrolled participants

| | Arm 1 (Z) | Arm 2 (DZ) | Arm 3 (EDZ) |
|---|---|---|---|
| ITT | n = 50 | n = 50 | n = 50 |
| Median Age, | 66 | 69 | 69 |
| years | (43, 84) | (45, 92) | (49, 83) |
| (range) | 28 | 34 | 35 |
| ≥65 years | (56) | (68) | (70) |
| Squamous cell | 9 | 16 | 16 |
| carcinoma, n (%) | (18) | (32) | (32) |
| Brain mets at | 7 | 8 | 8 |
| baseline, n (%) | (14) | (16) | (16) |
| Liver mets at | 9 | 11 | 4 |
| baseline, n (%) | (18) | (22) | (8) |

The co-primary endpoints for this study were objective response rate (ORR) and progression free survival (PFS). ORR was defined as the percentage of participants with a best overall response (BOR) of either compete response (CR) or partial response (PR). Participants with measurable disease at baseline who discontinued prior to their first scheduled scan were considered non-responders. PFS was defined from the time of randomization until first documentation of disease progression, or death from any cause, whichever occurred first. In addition to the co-primary endpoints, disease control rate (DCR, defined as the percentage of participants with a BOR of CR, PR, or stable disease (SD) for >6 months), duration of response (DoR, defined as the time from first documentation of disease response (CR or PR) until first documentation of confirmed progressive disease) and overall survival (OS, defined as the time from randomization until death due to any cause) were also evaluated as exploratory endpoints. Efficacy analyses were based on the intent-to-treat (ITT) population, which comprises all randomized participants. PFS and cORR for the ITT population at a planned interim analysis are shown in Table 2.

TABLE 2
PFS and cORR for ITT population

| | Arm 1 (Z) | Arm 2 (DZ) | Arm 3 (EDZ) |
|---|---|---|---|
| | n = 50 | n = 50 | n = 50 |
| cORR* | 30% | 40% | 44% |
| [95% CI] | [17.9, 44.6] | [26.4, 54.8] | [30, 58.7] |
| PFS | | | |
| Median, mos | 5.4 | 9.3 | 9.9 |
| [95% CI] | [2.7, 9.7] | [4.1, NE] | [4.8, 14.6] |
| Events (%) | 32 | 27 | 30 |
| | (64%) | (54%) | (60%) |
| HR vs. Z | — | 0.67 | 0.72 |
| [95% CI] | | [0.40, 1.13] | [0.44, 1.20] |

*One participant in Arm 2 had response pending confirmation, which was confirmed after the planned interim analysis data cut-off date. HR: Hazard Ratio, cORR: confirmed objective response rate; PFS: progression free survival; Z: zimberelimab, D: domvanalimab; E: etrumadenant; NE: not evaluable

As part of an exploratory analysis, clinical outcomes in biomarker selected subsets were investigated retrospectively. Central submission of adequate tumor tissue and central confirmation of PD-L1 status was not required for study enrollment. Thus, the number of biomarker evaluable patients (BEP) varied by biomarker and was less than the ITT population. Expression levels of CD155, CD226, and CD73 were measured in baseline tumor biopsy samples (e.g., formalin-fixed, paraffin-embedded tissue or freshly cut, serial unstained sections), along with baseline expression level of PD-L1. The baseline samples were archival tumor samples obtained ≤24 months prior to screening or fresh biopsy samples obtained prior to the first dose of treatment. PD-L1 expression level in the BEP subset was measured at a central lab using the Ventana SP263 IHC assay, while enrollment in the trial was based on PD-L1 expression (250% TC or TPS) locally assessed by Ventana SP263 assay or PharmDx 22C3 assay. CD155, CD226 and CD73 expression levels were measured at a central lab as described in Example L. PD-L1, CD155, CD226, and CD73 expression at baseline are summarized in Table 3. Median PFS and cORR in biomarker selected subsets of the biomarker evaluable population are shown in Table 4 and Table 5. The best predictive biomarker of activity in Arm 1 was CD155 (e.g., CD155 low). At least three biomarkers (e.g., PD-L1 high, CD155 high, and CD73 low) enrich for clinical activity of domvanalimab and there was an even greater enhancement in patients that fulfill two of the biomarkers (analyses not shown). Notably, benefit attributed to the anti-TIGIT antibody is concentrated onto those patients least likely to benefit from anti-PD-1 monotherapy (e.g., CD155 high subset and/or CD73 low subset).

TABLE 3
Baseline PD-L1, CD155, CD226, and CD73 expression levels

| | Arm 1 | Arm 2 | Arm 3 |
|---|---|---|---|
| | (Z) | (DZ) | (EDZ) |
| PD-L1 expression level | | | |
| BEP, n | 39 | 38 | 37 |
| PD-L1: ≥50% TC, n (%) | 24 | 27 | 28 |
| | (62) | (71) | (76) |
| CD73 expression level | | | |
| BEP | 36 | 31 | 27 |
| % TC, mean (SD), | 56.3 | 47.9 | 56.3 |
| | (40.8) | (41.4) | (44.7) |
| % TC, median (IQR) | 66.0 | 50.0 | 80.0 |
| | (11.2, 96.2) | (2.0, 90.0) | (0.0, 100.0) |
| % TC, range | 0, 100 | 0, 100 | 0, 100 |
| CD226 expression level | | | |
| BEP | 24 | 23 | 20 |
| % IC, mean (SD) | 5.3 | 7.2 | 12.4 |
| | (5.4) | (12.0) | (19.3) |
| % IC, median (IQR) | 5.0 | 1.0 | 5.0 |
| | (0.25, 10.0) | (0.0, 10.0) | (1.0, 20.0) |
| % IC, range | 0.0, 20.0 | 0.0, 50.0 | 0.0, 80.0 |
| CD155 expression level | | | |
| BEP | 34 | 31 | 28 |
| H-score, mean (SD) | 144.4 | 125.6 | 155.2 |
| | (82.6) | (80) | (84.4) |
| H-score, median (IQR) | 132.5 | 110.0 | 150.0 |
| | (90.0, 210.0) | (70.0, 185.0) | (97.5, 218.8) |
| H-score, range | 0, 290 | 0, 290 | 5, 290 |

TABLE 4
Median PFS and cORR in biomarker selected subsets
of the biomarker evaluable population

| Biomarker, | # | cORR | PFS, mo | Events | HR vs. Low | Prob > |
|---|---|---|---|---|---|---|
| Treatment Arm | BEP | % | (95% CI) | % | (95% CI) | Chiso |
| PD-L1 Low, 1 | 15 | 27 | 5.4 | 60 | 1.04 | 0.92 |
| | | | (1.38, NE) | | (0.46, 2.36) | |
| PD-L1 High, 1 | 24 | 33 | 5.7 | 67 | | |
| | | | (1.48, 9.66) | | | |
| PD-L1 Low, 2 | 11 | 18 | 6.1 | 73 | 0.59 | 0.24 |
| | | | (1.64, 9.66) | | (0.24, 1.43) | |
| PD-L1 High, 2 | 27 | 44 | 12.0 | 48 | | |
| | | | (3.25, NE) | | | |
| PD-L1 Low, 3 | 9 | 33 | 10.9 | 56 | 1.21 | 0.71 |
| | | | (1.28, 17.68) | | (0.42, 1.56) | |
| PD-L1 High, 3 | 28 | 43 | 7.6 | 64 | | |
| | | | (3.15, 12.98) | | | |
| PD-L1 Low, | 20 | 30 | 7.6 | 65 | 0.81 | 0.53 |
| 2 + 3 | | | (3.84, 13.86) | | (0.42, 1.56) | |
| PD-L1 High, | 55 | 42 | 9.9 | 56 | | |
| 2 + 3 | | | (4.01, 25.40) | | | |
| CD155 Low, 1 | 17 | 41 | 9.7 | 47 | 0.38 | 0.03* |
| | | | (1.68, NE) | | (0.16, 0.93) | |
| CD155 High, 1 | 17 | 24 | 3.5 | 82 | | |
| | | | (1.38, 4.47) | | | |
| CD155 Low, 2 | 18 | 44 | 12.0 | 44 | 0.47 | 0.13 |
| | | | (3.55, NE) | | (0.18, 1.25) | |
| CD155 High, 2 | 13 | 23 | 5.5 | 69 | | |
| | | | (1.08, NE) | | | |
| CD155 Low, 3 | 10 | 50 | 11.2 | 60 | 0.93 | 0.88 |
| | | | (1.28, NE) | | (0.34, 2.52) | |
| CD155 High, 3 | 18 | 28 | 7.6 | 67 | | |
| | | | (1.41, 25.40) | | | |
| CD155 Low, | 28 | 46 | 11.2 | 50 | 0.65 | 0.22 |
| 2 + 3 | | | (4.01, NE) | | (0.33, 1.29) | |
| CD155 High, | 31 | 26 | 5.5 | 68 | | |
| 2 + 3 | | | (2.69, 9.26 | | | |
| CD73 Low, 1 | 17 | 12 | 3.5 | 65 | 0.63 | 0.27 |
| | | | (1.28, 5.68) | | (0.28, 1.42) | |
| CD73 High, 1 | 19 | 42 | 9.6 | 68 | | |
| | | | (1.68, 14.82) | | | |
| CD73 Low, 2 | 18 | 50 | NE | 44 | 2.36 | 0.08 |
| | | | (4.01, NE) | | (0.91, 6.10) | |
| CD73 High, 2 | 13 | 23 | 3.5 | 77 | | |
| | | | (1.02, 11.99) | | | |
| CD73 Low, 3 | 12 | 42 | 13.0 | 58 | 0.98 | 0.96 |
| | | | (1.38, NE) | | (0.36, 2.62) | |
| CD73 High, 3 | 15 | 40 | 7.7 | 67 | | |
| | | | (1.41, 25.40) | | | |
| CD73 Low, | 30 | 47 | 13.0 | 50 | 1.65 | 0.15 |
| 2 + 3 | | | (3.84, NE) | | (0.84, 3.26) | |
| CD73 High, | 28 | 32 | 7.6 | 71 | | |
| 2 + 3 | | | (2.69, 11.99) | | | |
| CD226 Low, 1 | 11 | 27 | 2.7 | 73 | 0.473 | 0.21 |
| | | | (0.79, 9.56) | | (0.15, 1.52) | |
| CD226 High, 1 | 13 | 39 | 5.4 | 69 | | |
| | | | (1.81, 14.82) | | | |
| CD226 Low, 2 | 13 | 54 | 10.9 | 54 | 0.836 | 0.77 |
| | | | (2.60, NE) | | (0.25, 2.77) | |
| CD226 High, 2 | 10 | 30 | NE | 40 | | |
| | | | (1.02, NE) | | | |
| CD226 Low, 3 | 9 | 44 | 7.6 | 89 | 0.353 | 0.11 |
| | | | (1.28, 17.68) | | (0.10, 1.25) | |
| CD226 High, 3 | 10 | 40 | 13.0 | 40 | | |
| | | | (1.41, NE) | | | |
| CD226 Low, | 22 | 50 | 7.6 | 68 | 0.66 | 0.36 |
| 2 + 3 | | | (2.60, 17.68) | | (0.27, 1.59) | |
| CD226 High, | 20 | 35 | 13.0 | 40 | | |
| 2 + 3 | | | (4.07, NE) | | | |

HR: Hazard Ratio, cORR: confirmed objective response rate; PFS: progression free survival; PD-L1 Low: <50% TC, PD-L1 High: ≥50% TC; CD155 Low: H-score < 135, CD155 High: H-score ≥ 135; CD73 Low: <61% TC, CD73 High: ≥61% TC; CD226 Low: <4% IC, CD226 High: ≥4% IC; NE: not evaluable
*p value ≤ 0.05

TABLE 5
Median PFS and cORR in biomarker selected subsets
of the biomarker evaluable population

| Biomarker, | # | cORR | PFS, mo | Events | HR vs. Arm 1 | Prob> |
|---|---|---|---|---|---|---|
| Treatment Arm | BEP | (%) | (95% CI) | (%) | (95% CI) | Chisq |
| PD-L1 High Arm 1 | 24 | 33 | 5.7 | 67 | — | — |
| | | | (1.48, 9.66) | | | |
| PD-L1 High Arm 2 | 27 | 44 | 12.0 | 48 | 0.61 | 0.18 |
| | | | (3.25, NE) | | (0.29, 1.27) | |
| PD-L1 High Arm 3 | 28 | 43 | 7.6 | 64 | 0.86 | 0.66 |
| | | | (3.15, 12.98) | | (0.44, 1.69) | |
| CD155 High Arm 1 | 17 | 24 | 3.5 | 82 | — | — |
| | | | (1.4, 4.5) | | | |
| CD155 High Arm 2 | 13 | 23 | 5.5 | 69 | 0.76 | 0.53 |
| | | | (1.6, 9.3) | | (0.33, 1.78) | |
| CD155 High Arm 3 | 18 | 28 | 7.6 | 67 | 0.66 | 0.30 |
| | | | (3, NE) | | (0.31, 1.44) | |
| CD73 Low Arm 1 | 17 | 12 | 3.5 | 65 | — | — |
| | | | (1.28, 5.68) | | | |
| CD73 Low Arm 2 | 18 | 50 | NE | 44 | 0.34 | 0.02* |
| | | | (4.01, NE) | | (0.14, 0.87) | |
| CD73 Low Arm 3 | 12 | 42 | 13.0 | 58 | 0.50 | 0.15 |
| | | | (1.38, NE) | | (0.19, 1.30) | |
| CD226 High Arm 1 | 13 | 39 | 5.4 | 69 | — | — |
| | | | (1.81, 14.82) | | | |
| CD226 High Arm 2 | 10 | 30 | NE | 40 | 0.45 | 0.19 |
| | | | (1.02, NE) | | (0.14, 1.48) | |
| CD226 High Arm 3 | 10 | 40 | 13.0 | 40 | 0.62 | 0.43 |
| | | | (1.41, NE) | | (0.19, 2.04) | |

HR: Hazard Ratio, cORR: confirmed objective response rate; PFS: progression free survival; PD-L1 High: ≥50% TC; CD155 High: H-score ≥ 135; CD73 Low: <61% TC; CD226 High: ≥4% IC, NE: not evaluable
*p value ≤ 0.05

Optimal biomarker cut-offs (e.g., reference values) can be analyzed using the median values described above, or using quartiles and tertiles, as reference points. Statistical methods, including but not limited to receiver operating characteristic (ROC) analysis, can also be used to determine the cut-off of best fit, whereby the difference in clinical benefit between biomarker-high and biomarker-low populations is most significant. Additional datasets and descriptive statistics of biomarker distribution can also inform clinically relevant cut-off/threshold selection.

The biomarker analysis in this example suggests that the benefit attributed to an anti-TIGIT antibody, when used in combination with a PD-(L)1 antagonist, is concentrated on those patients least likely to benefit from PD-(L)1 monotherapy (e.g., CD155 high subset and/or CD73 low subset). Additional experiments are planned and in various stages of execution to test the hypothesis that these biomarkers (used individually or in combination) can identify cancers with differentiated immunological and biological attributes.

Briefly, IHC, efficacy, and transcriptomic and genomic analyses of tissue samples are planned or underway to iteratively assess for co-associating features (for example association of enhanced patient outcomes with a particular IHC expression profile (e.g., low CD73, high CD155, etc.) and/or corresponding transcriptomic signatures) that allow for prediction of how patients may respond to any given drug regimen and/or an indication of a range of drug-mediated molecular or cellular responses that may be used to presage patient outcomes (positive or negative). To do this, baseline and on-treatment tissue samples from additional subjects with cancer (e.g., participants enrolled in the trials described in Examples 3 ad 4, or tissue samples obtained from commercial sources) are being profiled to evaluate expression levels and spatial distribution of analytes of interest (for example PD-L1, CD73, PVR, DNAM etc.), as described herein. Tissue IHC scores for individual biomarkers and combinations thereof will again be analyzed by patient treatments and outcomes. Nucleic acid (RNA and DNA) is also being prepared from these same tumor tissues, and also from blood samples obtained from the same participants. Nucleic acids in the samples can be sequenced using standard next generation sequencing approaches and analyzed for a variety of features to include, but not limited to, tumor mutational burden (TMB), microsatellite instability (MSI) and mutational status. Additional analysis will focus on identifying and assessing the transcriptional profiles of individual patient tissue or blood samples for features, fingerprints and profiles that may predict response or drug (drug-combination)-dependent effects or changes (for example, gene signatures indicative of immune infiltration, adenosine pathway modulation or immune activation etc.). Cytokines are also being measured in these tumor and blood samples including, but not limited to, CXCL-9, CXCL-10, CXCL-11, IL-18, IFN Gamma, TNF Alpha, IL-12B.

### Example 3

In Example 2, CD155 expression was measured in tumor tissue from a subset of the intent to treat population of a clinical trial. Stained slides were evaluated by a pathologist who scored CD155 staining intensity on a semi-quantitative scale: 0=negative, 1⁺=weak intensity, 2⁺=moderate intensity, and 3+=strong intensity, and then H-scores were calculated. In this example, alternative scoring methods were tested.

Without rescoring CD155 intensity (i.e., using the original values scored by the pathologist), expression was characterized by two different scoring algorithms—namely, % TC and % TC 2-3+. The coefficient of determination (R² value) was used to look at the variance between each approach and CD155 expression characterization by H-scores. While % TC 2-3+ and H-score are highly concordant (R² value of 0.94), less concordance was observed between % TC and H-score (R² value of 0.71). Samples were then categorized as “high” or “low” using as a cut-off value either a 2+ or 3+ % TC of 40% or a tumor H-score of 135, whereby a sample was CD155 high when the sample had CD155 expression equal to or greater than the cut-off value. Five samples were categorized differently between the two approaches—two were CD155 high when the cut-off value was 2+ or 3+ % TC of 40% but low when the cut-off value was a tumor H-score of 135, and three were CD155 high when the cut-off value was a tumor H-score of 135 but low when the cut-off was 2+ or 3+ % TC of 40%—resulting in a 94.6% overall agreement. When a tumor H-score of 140 was used as the cut-off rather than a tumor H-score of 135, only two samples were categorized differently—both were CD155 high when the cut-off value was 2+ or 3+ % TC of 40% but low when the cut-off value was a tumor H-score of 140—resulting in a 97.8% overall agreement. Survival curves were created with JMP software (version 17.0) using the Kaplan-Meier method and the Cox proportion hazards model was used to estimate the HR for progression-free survival and their 95% CI. See Table 6.

TABLE 6

| | Treatment | | cORR | PFS, mo | Event | HR |
|---|---|---|---|---|---|---|
| Population | Arm | N | (%) | [95% CI] | Rate (%) | [95% CI] |
| BEP | Arm 1 (Z) | 34 | 32 | 5.4 | 65 | |
| | | | | [1.7, 9.7] | | |
| | Arm 2 (DZ) | 31 | 35 | 9.3 | 55 | 0.76 |
| | | | | [3.5, NE] | | [0.44, 1.44] |
| | Arm 3 (EDZ) | 28 | 36 | 7.6 | 64 | 0.89 |
| | | | | [3, 17.7] | | [0.48, 1.66] |
| CD155 Low | Arm 1 (Z) | 17 | 41 | 9.7 | 47 | |
| (H-score < 135) | | | | [1.7, NE] | | |
| | Arm 2 (DZ) | 18 | 44 | 12 | 44 | 0.87 |
| | | | | [3.5, NE] | | [0.33, 2.33] |
| | Arm 3 (EDZ) | 10 | 50 | 11.2 | 60 | 1.24 |
| | | | | [1.3, NE] | | [0.43, 3.58] |
| CD155 High | Arm 1 (Z) | 17 | 24 | 3.5 | 82 | |
| (H-score ≥ 135) | | | | [1.38, 4.47] | | |
| | Arm 2 (DZ) | 13 | 23 | 5.5 | 69 | 0.76 |
| | | | | [1.08, NE] | | [0.33, 1.78] |
| | Arm 3 (EDZ) | 18 | 28 | 7.6 | 67 | 0.66 |
| | | | | [1.41, 25.4] | | [0.31, 1.44] |
| CD155 Low | Arm 1 (Z) | 17 | 47 | 9.7 | 47 | |
| (2+ or 3+ | | | | [1.7, NE] | | |
| % TC < 40%) | Arm 2 (DZ) | 18 | 39 | 10.9 | 50 | 0.98 |
| | | | | [3.5, NE] | | [0.38, 2.54] |
| | Arm 3 (EDZ) | 11 | 45 | 11.2 | 64 | 1.38 |
| | | | | [1.4, NE] | | [0.5, 3.82] |
| CD155 High | Arm 1 (Z) | 17 | 18 | 3.5 | 82 | |
| (2+ or 3+ | | | | [1.38, 4.47] | | |
| % TC ≥ 40%) | Arm 2 (DZ) | 13 | 31 | 5.5 | 62 | 0.66 |
| | | | | [1.08, NE] | | [0.27, 1.58] |
| | Arm 3 (EDZ) | 17 | 29 | 7.6 | 65 | 0.61 |
| | | | | [1.41, 25.4] | | [0.27, 1.35] |

### Example 4

The potential treatment benefit of zimberelimab and domvanalimab combination therapy is being evaluated in people with histologically confirmed, treatment naïve, PD-L1 high, squamous or non-squamous NSCLC that is locally advanced or metastatic (Stage IIIB-IV per American Joint Committee on Cancer, version 8) without actionable mutation. Actionable mutations include those mutations for which a targeted therapy is approved by local health authority and available at the time of enrollment (e.g., ALK fusion oncogene, certain EGFR, ROS, BRAF, NTRK mutations).

In Part 1 of the study, three arms were enrolled to evaluate the safety and efficacy of zimberelimab alone vs. zimberelimab plus domvanalimab vs. platinum doublet chemotherapy. Participants in Arm 1 received zimberelimab 360 mg IV Q3W until disease progression or intolerance. Participants in Arm 2 received domvanalimab 15 mg/kg IV Q3W and zimberelimab 360 mg IV Q3W until disease progression or intolerance. Participants in Arm 3 received carboplatin AUC 5 or 6 (maximum dose 900 mg) plus either paclitaxel 200 mg/m2 IV Q3W or pemetrexed 500 mg/m2 IV Q3W until disease progression, at which time they could crossover to Arm 1. PD-L1 high status (tumor proportion score (TPS) ≥50%) as determined by the 22C3 PharmDx assay was assessed and confirmed by central laboratories.

In Part 2 of the study, enrollment in Part 1 stopped and two new arms enrolled to evaluate the safety and efficacy of zimberelimab plus domvanalimab vs. pembrolizumab. Participants in Arm 4 receive zimberelimab 360 mg by IV infusion Q3W plus domvanalimab 1200 mg by IV infusion Q3W on Day 1 of each 21-day cycle until disease progression or intolerance. Participants in Arm 5 receive pembrolizumab 200 mg by IV infusion Q3W on Day 1 of each 21-day cycle until disease progression or intolerance. Approximately 300 participants may be randomized to each of Arm 4 and Arm 5. Randomization is stratified based on Eastern Cooperative Oncology Group performance status (ECOG PS; 0 or 1), geographic region (Asia vs. non-Asia), and histology (squamous vs. non-squamous). PD-L1 high status (tumor cell (TC) ≥50%) as determined by the Ventana SP263 IHC assay is assessed by central laboratories.

Participants already enrolled and assigned to Part 1 treatment arms may continue on study until disease progression, withdrawal of consent, or unacceptable toxicity. The total number of participants in Part 1 is estimated to be approximately 125 with 50 participants each in Arm 1 and Arm 2, and 25 participants in Arm 3. In addition, crossover from Arm 3 to Arm 1 is still permitted.

The primary endpoint for this study is overall survival (OS) in the intent to treat population, which comprises all randomized participants in Part 2. Secondary endpoints include but are not limited to progression free survival (PFS) and confirmed objective response rate (cORR). As part of an exploratory analysis, clinical outcomes in biomarker selected subsets are investigated retrospectively to understand the disease biology, response to treatment, and to identify surrogate markers of response. Exploratory biomarker analyses include but are not limited to analysis of protein expression levels (e.g., PD-L1, CD155, CD73, cytokines, etc.) and may also include NGS, RNAseq (immune context), WES, and/or ctDNA analyses to determine microsatellite instability status (stable, low, or high), tumor mutational burden, mutational landscape, and transcriptome information. Analyses may occur at baseline, on study, and/or at time of progression. The number of biomarker evaluable patients (BEP) varies by biomarker and may be less than the ITT population. Protein expression levels of CD155 and CD73 are measured in baseline tumor biopsy samples where available (e.g., formalin-fixed, paraffin-embedded tissue, a fresh biopsy sample, or freshly cut, serial unstained 4-5 m sections), along with baseline expression level of PD-L1. The baseline samples are archival tumor samples obtained ≤6 months prior to screening or fresh biopsy samples obtained prior to the first dose of treatment. PD-L1 expression level is measured at a central lab using the Ventana SP263 IHC assay. CD155 and CD73 expression levels are measured at a central lab as described in Example 1. Whole blood samples are collected pre-dose at Cycle 1 Day 1, and then again at one or more times on study, and then again at the end of study treatment.

Expression levels of CD155 and PD-L1 were measured in baseline tumor biopsy samples when available. PD-L1 expression level was measured at a central lab for enrollment in the trial using the PharmDx 22C3 assay and/or Ventana SP263 IHC assay (PD-L1 expression of >50% TPS or TC). CD155 expression levels were measured at a central lab as described in Example 1. CD155 expression levels were also measured at the transcript level using bulk tumor RNAseq whereby tissue was macrodissected from FFPE microscope slides to enrich for tumor content, DNA and RNA was extracted, assessed for quality, a library was prepared, and used to sequence the whole transcriptome. PD-L1 and CD155 expression at baseline are summarized in Table 7.

TABLE 7

| | CD155 | | PD-L1 |
|---|---|---|---|
| | 2+ or | tumor | SP263 |
| | 3+ % TC | H-score | % TC |
| Number of values | 80 | 80 | 80 |
| Minimum | 0 | 0 | 0 |
| 25% percentile | 20 | 80 | 50 |
| Median | 40 | 132 | 75 |
| 75% percentile | 60 | 188 | 90 |
| maximum | 100 | 290 | 100 |
| 95% CI of median | | | |
| Low confidence level | 30 | 110 | 65 |
| Upper confidence level | 50 | 140 | 80 |

### Example 5

The potential treatment benefit of zimberelimab monotherapy vs. zimberelimab combination therapy with an anti-TIGIT antibody is being evaluated in 1L and ≥2L advanced GI malignancies. Arm 1 evaluates 1L treatment in immune checkpoint inhibitor (CPI) naïve participants without prior systemic therapy that have locally advanced or metastatic disease and measurable (RECIST 1.1) EAC, GEJ or gastric cancer. Participants receive (a) domvanalimab 1600 mg Q4W, zimberelimab 480 mg Q4W and FOLFOX (oxaliplatin 85 mg/m², leucovorin 400 mg/m², fluorouracil 400 mg/m² Day 1 and fluorouracil 1200 mg/m² D1-D2, Q2W) or (b) zimberelimab 480 mg Q4W in addition to chemotherapy with FOLFOX. Arm 2 evaluates ≥2L treatment in CPI naïve participants with locally advanced or metastatic disease and measurable (RECIST 1.1) EAC, GEJ or gastric cancer. Participants receive (a) domvanalimab 1200 mg Q3W and zimberelimab 360 mg Q3W, (b) quemliclustat 100 mg Q2W and zimberelimab 480 mg Q4W, or (c) quemliclustat 100 mg Q2W, domvanalimab 1600 mg Q4W and zimberelimab 480 mg Q4W. Arm 3 evaluates ≥2L treatment in CPI experienced participants with locally advanced or metastatic disease and measurable (RECIST 1.1) EAC, GEJ or gastric cancer. Participants receive domvanalimab 1200 mg Q3W and zimberelimab 360 mg Q3W. Enrolled participants have an ECOG performance score of 0-1. In Arm 1 only, participants with known HER-2 positive tumors are excluded from enrollment.

PD-L1 expression is monitored as participant accrual occurs and prospective testing of PD-L1 status may be required for participant selection if there is a significant imbalance between participants with high and low PD-L1 expression.

Participants continue treatment until the participant experiences an intolerable AE, participant withdraws consent to participate, pregnancy, noncompliance, use of another anticancer therapy, symptomatic deterioration attributed to disease progression, radiographically confirmed disease progression per RECIST 1.1, death, or the sponsor terminates the study, whichever occurs first.

The co-primary endpoints for this study are safety and clinical activity, measured by objective response rate (ORR). ORR is defined as the percentage of participants with a confirmed best response of either compete response (CR) or partial response (PR) as measured by RECIST 1.1 and assessed by the investigator. In addition to the co-primary endpoints, disease control rate (DCR, defined as the percentage of participants with a best overall confirmed response of CR, PR, or stable disease (SD) for ≥12 weeks from start of study intervention until disease progression), duration of response (DoR, defined as the time from first documentation of disease response (CR or PR) until first documentation of confirmed progressive disease or death, whichever comes first), progression free survival (PFS, defined from date of first dose until first documentation of disease progression, or death from any cause, whichever occurred first). and overall survival (OS, defined as the time from randomization until death due to any cause) were also evaluated as exploratory endpoints.

As part of an exploratory analysis, clinical outcomes in biomarker selected subsets are investigated retrospectively. The number of biomarker evaluable patients (BEP) varies by biomarker and is less than the ITT population. Expression levels of CD155 and CD73 are measured in baseline tumor biopsy samples (e.g., formalin-fixed, paraffin-embedded tissue or freshly cut, serial unstained sections), along with baseline expression level of PD-L1. The baseline samples are archival tumor samples obtained ≤12 months prior to screening or fresh biopsy samples obtained prior to the first dose of treatment. PD-L1 expression level is measured at a central lab using the Ventana SP263 IHC assay. CD155 and CD73 expression levels were measured at a central lab as described in Example 1. Exploratory biomarker analyses may also include NGS, RNAseq (immune context), WES, and/or ctDNA analyses to determine microsatellite instability status (stable, low, or high), tumor mutational burden, mutational landscape, and transcriptome information. CD155 and PD-L1 expression levels measured in subjects enrolled in Arm 1(a) and 1(b) and Arm (2) are provided in Table 8. There was a strong correlation between cytomembranous and membranous H-scores (Spearman r=0.8989, two-tailed P value=<0.0001) despite different medians.

TABLE 8

| | CD155 | | | | | |
|---|---|---|---|---|---|---|
| | | | | | Tumor area | |
| | Cyto-mem- | | Mem- | | covered by | PD-L1 |
| | branous | Cyto-mem- | branous | Mem- | CD155+ | SP263 |
| | 2+ or 3+ | branous | 2+ or 3+ | branous | immune | TAP |
| | % TC | H-score | % TC | H-score | cells (%) | (%) |
| Number of values | 56 | 56 | 56 | 56 | 56 | 56 |
| Minimum | 0 | 2 | 0 | 0 | 0 | 0 |
| 25% percentile | 3 | 85 | 3 | 10 | 2 | 1 |
| Median | 10 | 105 | 10 | 39 | 3 | 3 |
| 75% percentile | 50 | 153 | 50 | 123 | 4 | 5 |
| Maximum | 100 | 278 | 96 | 272 | 10 | 90 |
| 95% CI of median | | | | | | |
| Low confidence | 8 | 100 | 8 | 22 | 2 | 2 |
| level | | | | | | |
| Upper confidence | 18 | 115 | 15 | 50 | 3 | 5 |
| level | | | | | | |

### Example 6

Correlative clinical data and multiple mechanistic preclinical studies have pointed toward antigen-experienced T cells expressing low-to-moderate levels of PD-1 and TIGIT as mediators of anti-tumor immunity downstream of checkpoint blockade (Dolina et al., 2021, Frontiers in Immunology, 12(July), pp. 1-13). This heterogenous cell population has been characterized in the context of cancer and chronic viral infection to be dysfunctional or exhausted relative to canonical memory cells, and has equally heterogeneous nomenclature, being referred to as stem-cell memory, stem-like, progenitor, exhausted, or pre-dysfunctional, and herein will be collectively referred to as precursor-exhausted T cells (Tₚₑₓ) (Kallies, Zehn and Utzschneider, 2020, Nature Reviews Immunology, 20(2), pp. 128-136). Among the total exhausted T cell (Tₑₓ) population, Tₚₑₓ represent a self-renewing population at an early phase of differentiation, and activation of these cells leads to proliferation and mobilization. Through a series of intermediate effector phenotypes, Tₚₑₓ eventually differentiate to an epigenetically fixed terminally exhausted state (Tₜₑₓ) (Hudson et al., 2019, Immunity, 51(6), pp. 1043-1058; Beltra et al., 2020, Immunity, 52(5), pp. 825-841.e8; Dolina et al. Frontiers in Immunology, 12(July), pp. 1-13, 2021; Belk, Daniel and Satpathy, 2022, Nature Immunology, 23(6), pp. 848-860).

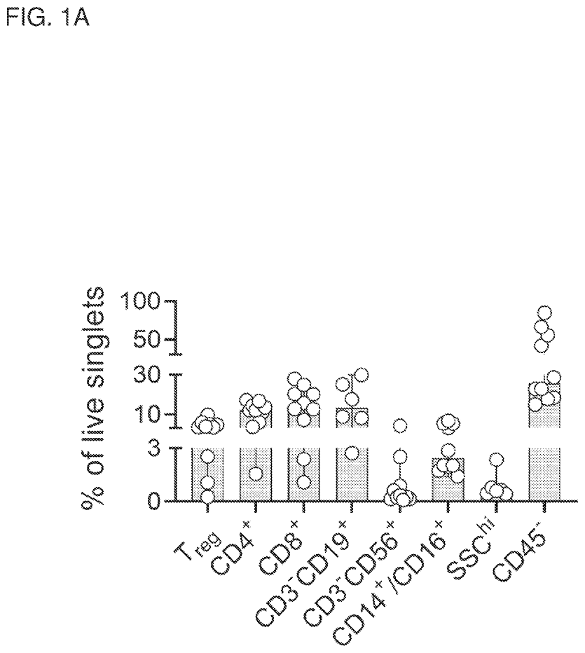

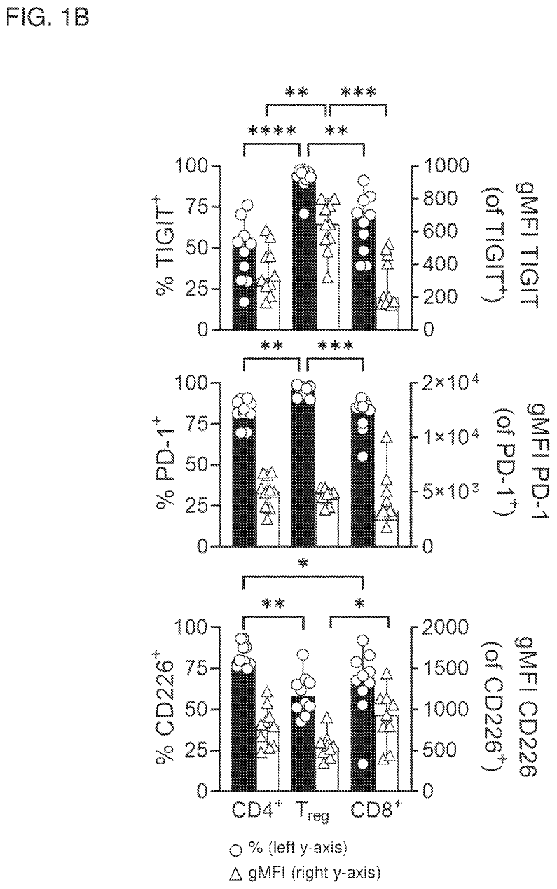

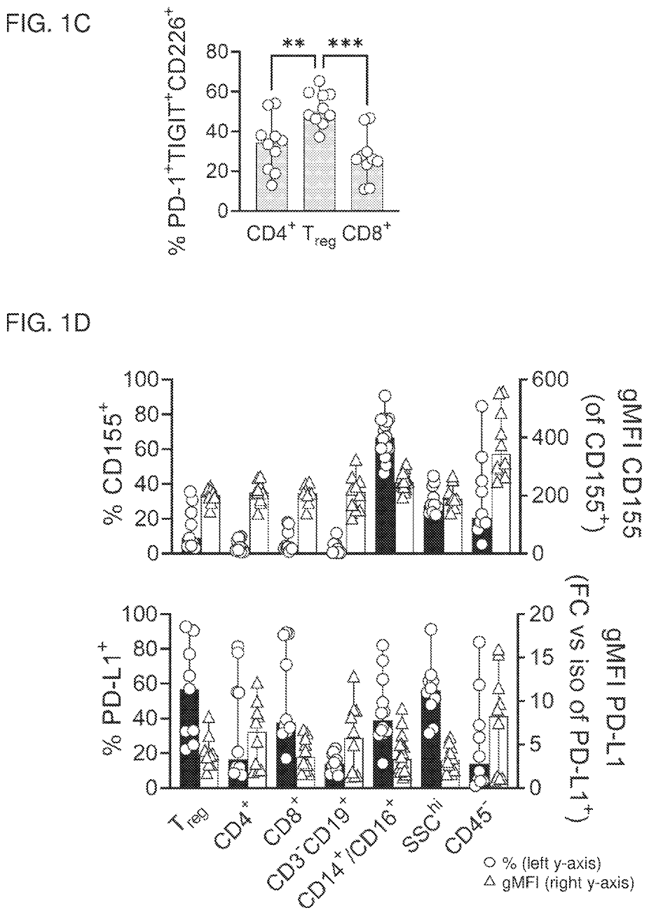

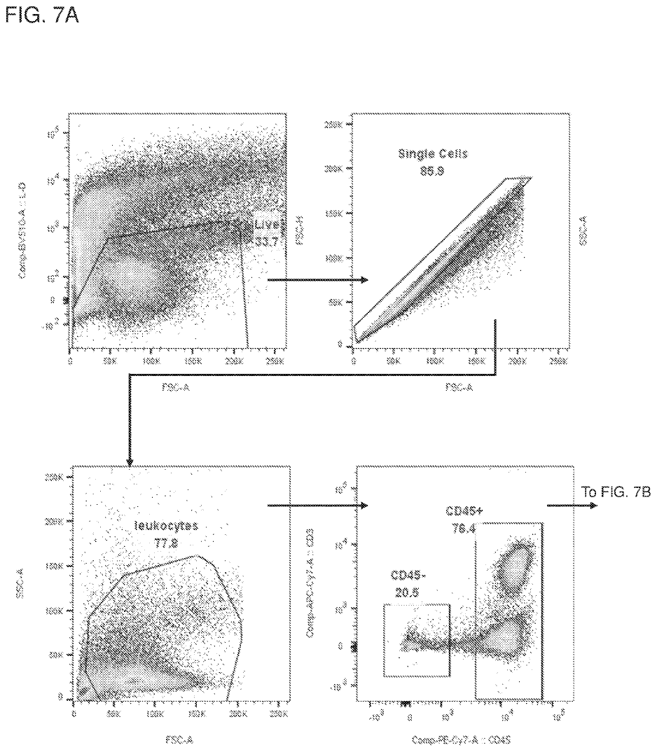

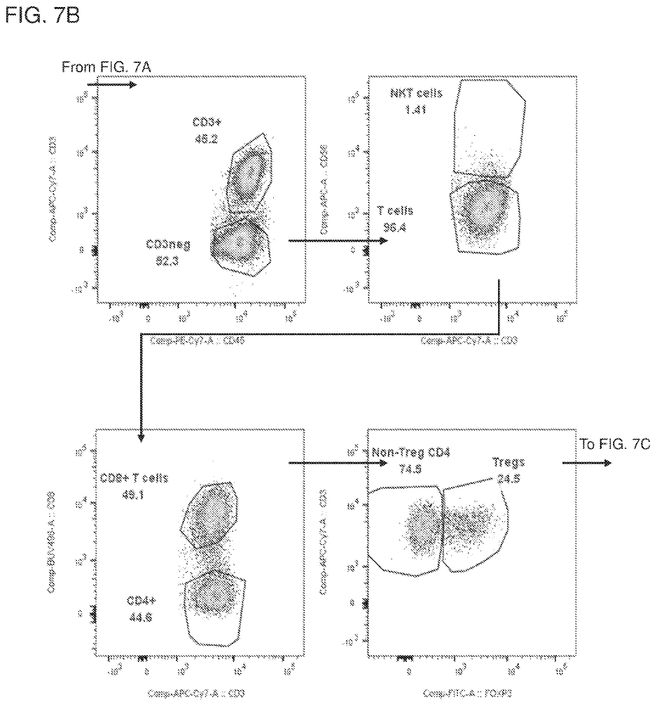

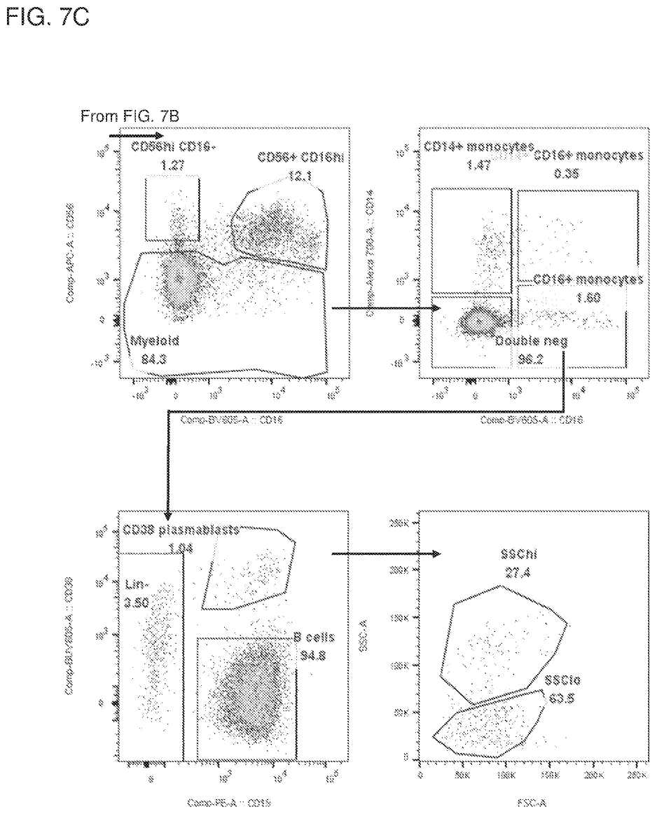

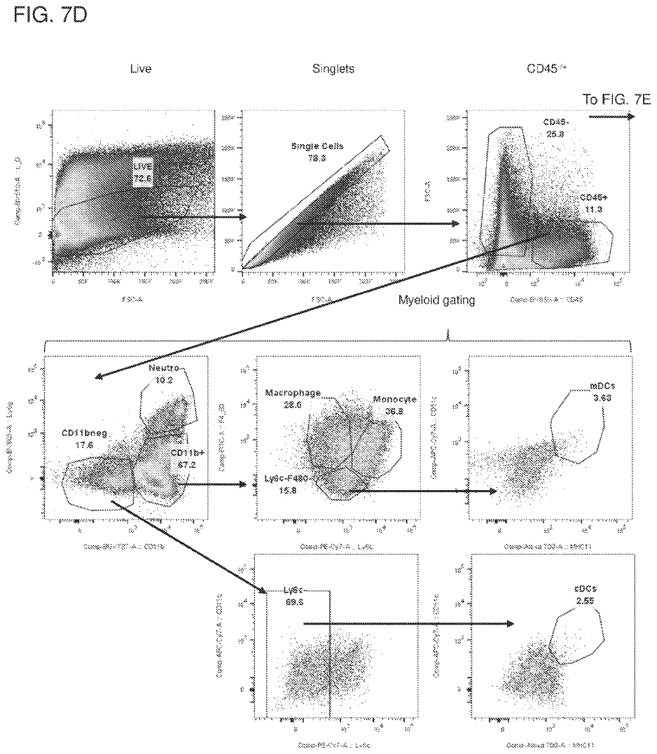

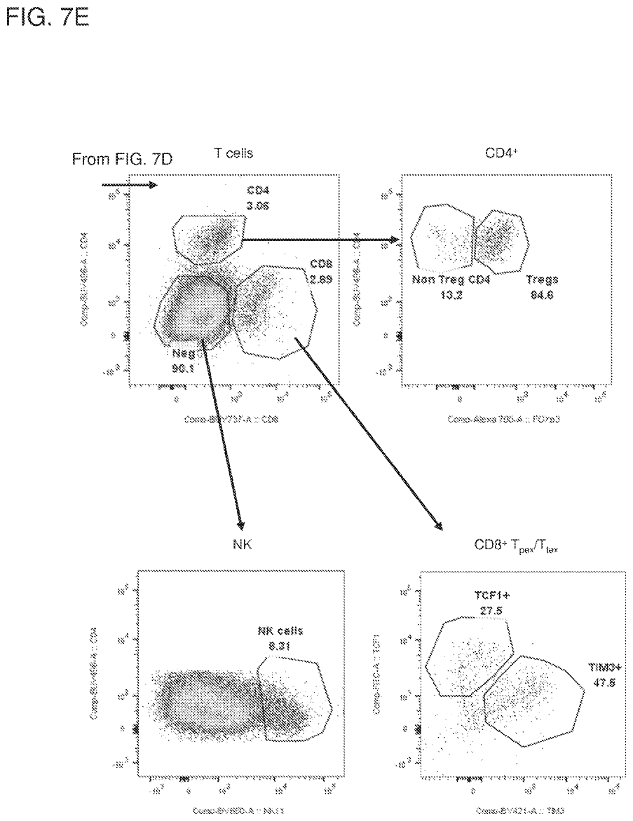

To understand the landscape of TIGIT and PD-1 receptor and related ligand expression in lung cancer, frozen single cell suspensions from NSCLC tumors were thawed and interrogated by flow cytometry. Compositionally, these samples consisted of broad T cell (regulatory T cells (T_{reg}), CD4⁺ non-T_{reg}, CD8⁺), B cell, and CD45⁻ subsets (representing cancer and stromal cells), with NK and myeloid lineages making up a minority fraction (FIG. 1A and FIG. 7A to FIG. 7C). TIGIT and PD-1 were expressed on all three major T cell populations, with the highest frequencies detected on the T_{reg} subset (FIG. 1B). On average, ~60-80% of each subset stained positive for CD226 (FIG. 1B), with ~25-50% being PD-1⁺TIGIT⁺CD226⁺ triple positive (FIG. 1C). Regarding primary receptor ligands, CD155 was principally expressed by myeloid subsets (and on the CD45⁺ population in some donors), while PD-L1 expression was more variable between donors and cell subsets (FIG. 1D).

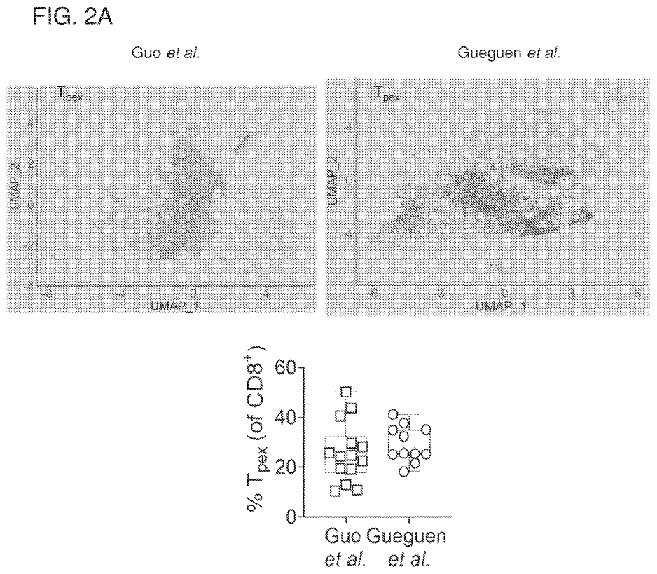

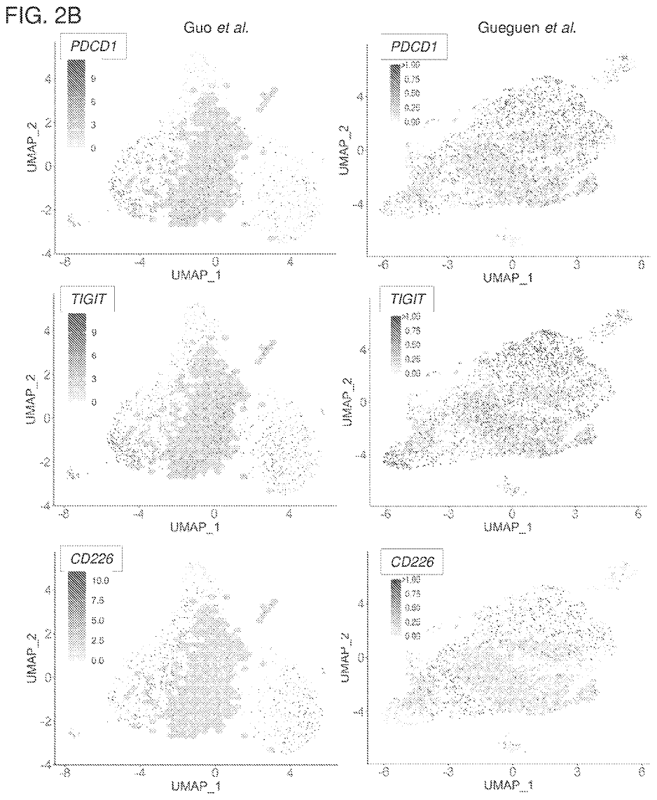

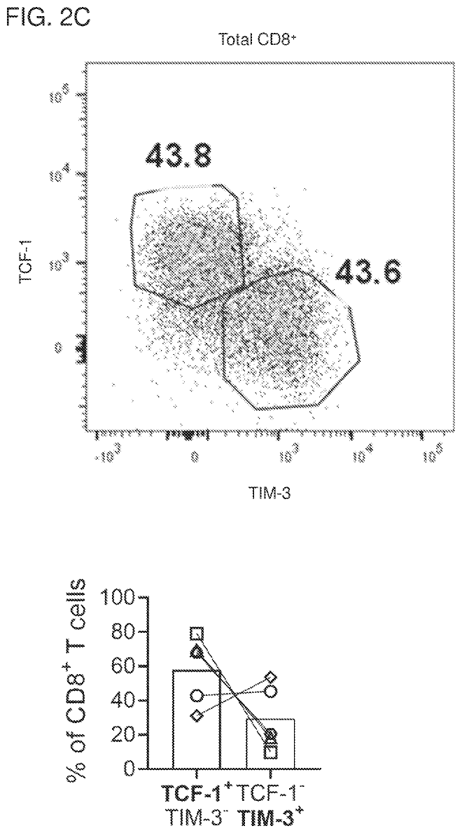

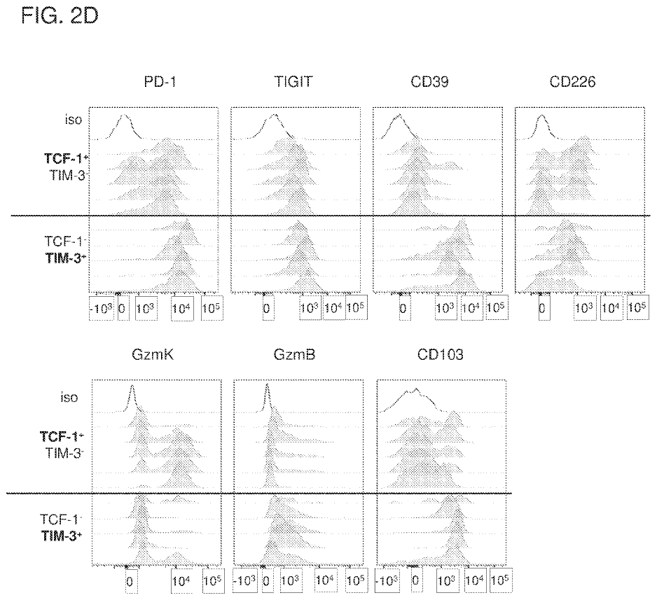

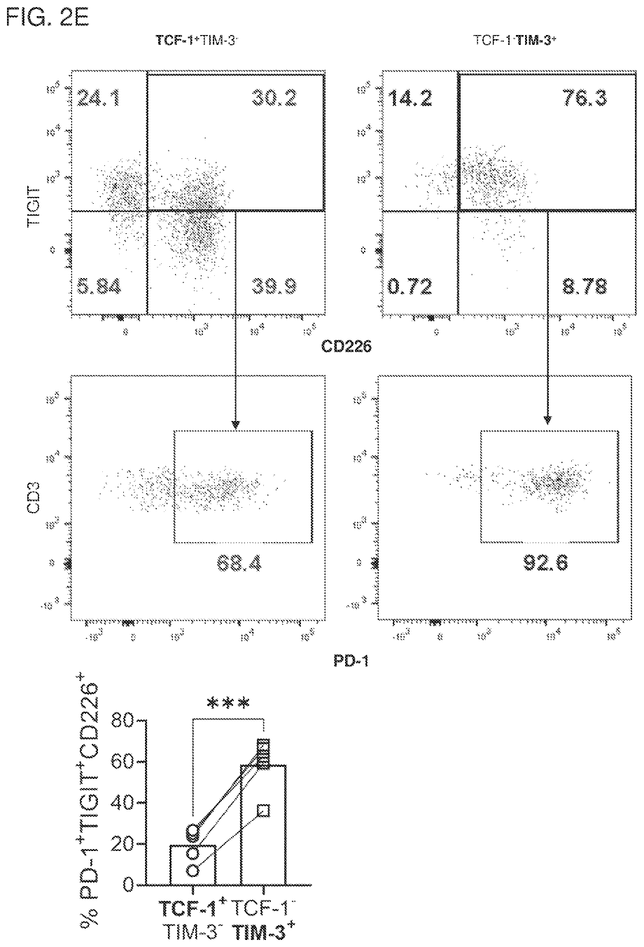

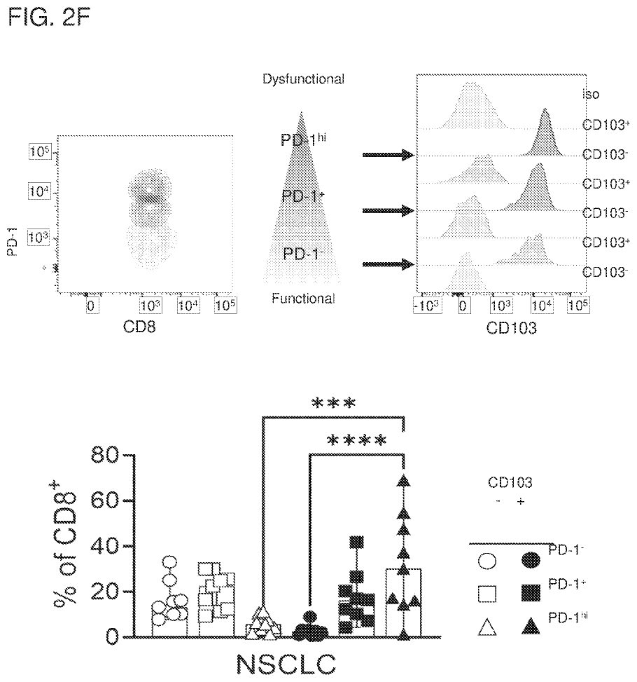

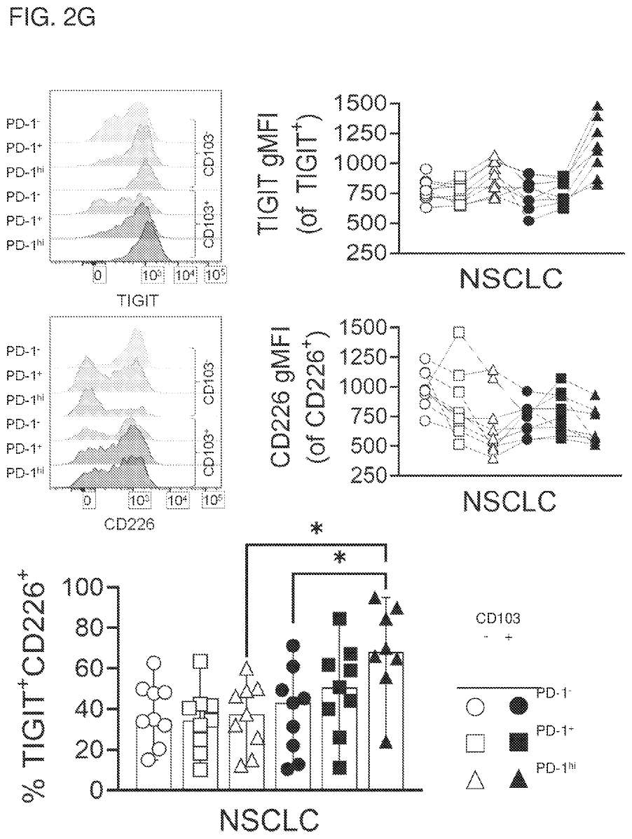

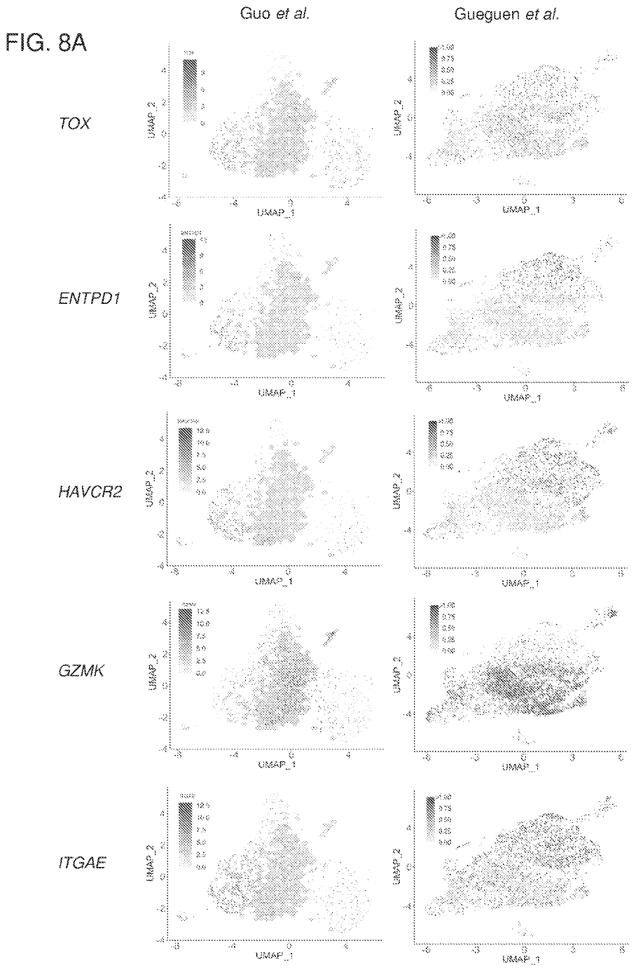

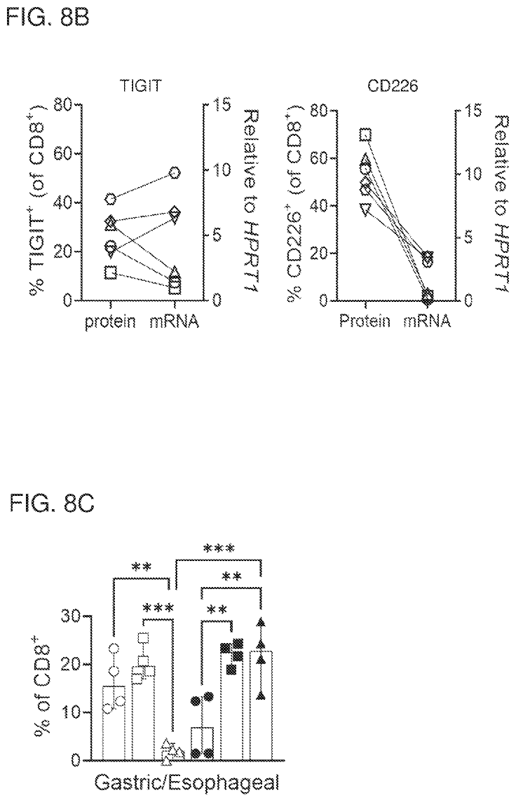

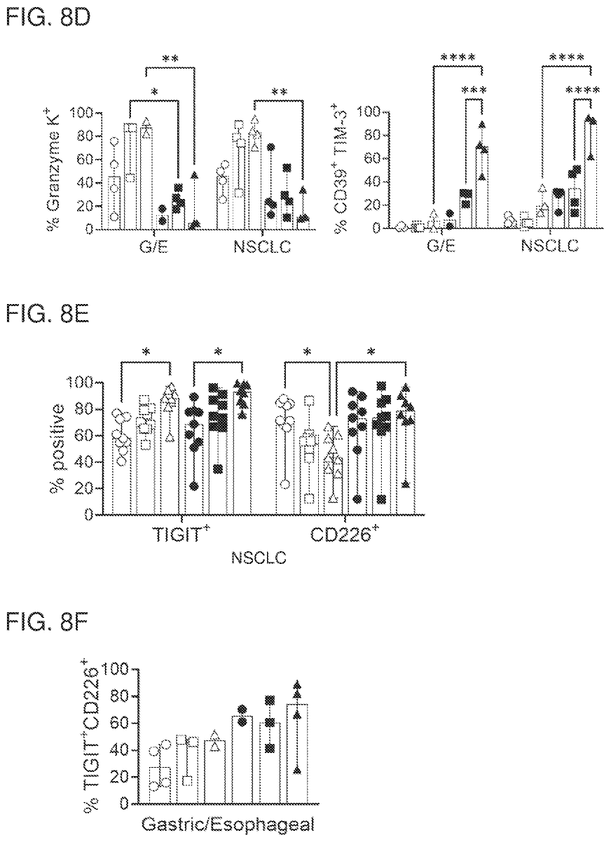

Focusing on CD8⁺ Tₑₓ subsets, two publicly available NSCLC single cell RNA-sequencing (scRNAseq) datasets were first leveraged (Guo et al., 2018, Nature Medicine, 24(7), pp. 978-985; Gueguen et al., 2021, Science Immunology, 6(55), p. eabd5778). Cells bearing an expression profile that discriminates a circulating Tₚₑₓ population (medium to high PDCD1, negative HAVCR2, positive GZMK) (Brummelman et al., 2018, The Journal of experimental medicine, 215(10), pp. 2520-2535; Chu, Berner and Zehn, 2020; Galletti et al., 2020; Gueguen et al., 2021, Science Immunology, 6(55), p. eabd5778, Nature Immunology, 21(12), pp. 1552-1562; Liu et al., 2022, Nature Cancer, 3(1), pp. 108-121) were present in both datasets (FIG. 2A, FIG. 2B and FIG. 8A). This population had appreciable expression of PD-1 and TIGIT; however, CD226 appeared strikingly low or absent (FIG. 2A, FIG. 2B). To corroborate these findings at the protein level, expression of TIGIT, PD-1, and CD226 was assessed on NSCLC tumor suspensions by flow cytometry using a gating scheme that classifies CD8⁺ Tₑₓ cells based on near mutually exclusive expression of the transcription factor TCF-1 and the immune checkpoint receptor TIM-3 (Miller et al., 2019, Nature Immunology, 20(3), pp. 326-336; Zehn et al., 2022, Nature Immunology. doi: 10.1038/s41590-022-01219-w; Prokhnevska et al., 2023, Immunity, 56(1), pp. 107-124.e5) (FIG. 2C). Histogram profiles for PD-1, Granzyme K/GzmK, and CD103 indicated that the TCF-1⁺TIM-3⁻ group was heterogenous, likely encompassing Tₚₑₓ, classical memory, and naïve subsets that are circulating (CD103⁻) or mucosal tissue-resident (CD103⁺) (FIG. 2D). PD-1, CD39, and CD103 protein expression was highest in the TCF-1⁻TIM-3⁺ cells, and this population also stained positive for Granzyme B (GzmB), suggestive of cells with effector potential amid tumor resident Tₜₑₓ clones (FIG. 2D). Co-expression of PD-1, TIGIT, and CD226 was significantly higher in the TCF-1⁻TIM-3⁺ subset relative to the TCF-1⁺TIM-3⁻ subset, consistent with the notion of increased checkpoint receptor expression on more differentiated Tₑₓ cells (FIG. 2E). Finally, in contrast to the scRNA-seq datasets, CD226 was detected on the surface of both subsets (FIG. 2D and FIG. 2E). In line with this discrepancy, CD226 mRNA levels were not indicative of relative protein levels when compared to TIGIT mRNA and protein in peripheral CD8⁺ T cells from healthy donors (FIG. 8B).

Using both NSCLC and gastroesophageal (G-E) tumor suspensions, CD8⁺ Tₑₓ populations were also analyzed using a more granular gating scheme that discriminates based on negative\positive\high PD-1 expression and negative\positive CD103 expression, resulting in six subsets representing various states of dysfunction and tissue residency (Duhen et al., 2018, Nature Communications, 9(1); Thommen et al., 2018, Nature Medicine, 24(7), pp. 994-1004) (FIG. 2F and FIG. 8C). Canonically, CD103^{-/+}PD-1⁺ cells include Tₚₑₓ and intermediate effector populations, whereas Tₜₑₓ cells reside in the CD103⁺PD-1^{hi} subset (Duhen et al., 2018, Nature Communications, 9(1); Thommen et al., 2018, Nature Medicine, 24(7), pp. 994-1004; Gueguen et al., 2021, Science Immunology, 6(55), p. eabd5778; Liu et al., 2022, Nature Cancer, 3(1), pp. 108-121). Consistent with sustained antigenic exposure in the tumor microenvironment (TME) driving terminal differentiation and checkpoint receptor expression (Kallies, Zehn and Utzschneider, 2020, Nature Reviews Immunology, 20(2), pp. 128-136; Budimir et al., 2022, Cancer Immunology Research, pp. 146-153), PD-1^{hi} cells were enriched in the CD103⁺ fraction and infrequent in the CD103⁻ subset; conversely, PD-1-cells were rare in tumor resident CD103⁺ fractions (FIG. 2F and FIG. 8C. In agreement with our scRNAseq Tₚₑₓ classification (FIG. 2A, FIG. 2B, and FIG. 8A), GzmK was enriched in CD103-PD-1^{+/hi} subsets relative to CD103⁺ counterparts in both NSCLC and G-E datasets, and similar to TCF-1⁻TIM-3⁺ cells, high levels of CD39 and TIM-3 were observed on CD103⁺PD-1^{hi} cells, supporting enrichment of a Tₜₑₓ population in the resident PD-1^{hi} cell fraction (FIG. 8D). In both the circulating and tissue resident fractions, PD-1^{hi} cells expressed the highest frequencies and intensities of TIGIT (FIG. 2G and FIG. 8E). Additionally, all three CD103⁺ tumor-resident PD-1 subsets displayed similarly high levels of CD226 expression (FIG. 2G and FIG. 8E). A considerable proportion of each subset co-expressed TIGIT and CD226, with CD103⁺PD-1^{hi} Tₜₑₓ cells exhibiting the highest co-expression frequencies (FIG. 2G and FIG. 8F), akin to TCF-1⁻TIM-3⁺ discriminated cells (FIG. 2E). While it should be emphasized that the subsets described here represent heterogenous T cell populations with various effector capabilities and differentiation states, these data indicate the receptors necessary to achieve benefit from PD-1 and TIGIT blockade are expressed on a large fraction of CD8⁺ T cells displaying tumor antigen-experienced phenotypes in NSCLC and G-E tumors.

Methods—Human tumor-infiltrating lymphocyte flow cytometry. Dissociated tumor biopsies were obtained from Discovery Life Sciences with informed written consent and according to Institutional Review Board (IRB)-approved guidelines in accordance with the Declaration of Helsinki. Subject information is detailed in Table 10. Tumor samples were thawed and washed with PBS, blocked with Human Fc Block (BD Biosciences), and stained with fluorophore-conjugated antibodies at 4° C. Cells were fixed/permeabilized using the FoxP3 transcription factor fixation kit (eBiosciences) and stained intracellularly at 4° C. Cells were washed and resuspended in PBS for flow cytometric analysis. Human antibodies used for flow cytometry are summarized in Table 9.

TABLE 9
Antibodies used for flow cytometry analysis

| Reactivity | Antibody | Conjugate | Clone | Vendor | Experiment |
|---|---|---|---|---|---|
| Mouse | CD39 | PerCp5.5710 | 24DMS1 | eBiosciences | MC38 |
| | CD4 | BUV496 | RM4-5 | BD | MC38 |
| | CD44 | BUV805 | IM7 | BD | MC38 |
| | CD45 | BV786 | 30-F11 | BD | MC38 |
| | CD8 | BUV737 | 53-6.7 | BD | MC38 |
| | NK-1.1 | BV650 | PK136 | BD | MC38 |
| | PD-1 | BUV395 | J43 | BD | MC38 |
| | TCF1 | AF488 | C63D9 | Cell Signaling | MC38 |
| | | | | Technologies | |
| | | | | (CST) | |
| | TIM3 | BV421 | RMT3-23 | BD | MC38 |
| | CD69 | BV510 | H1.2F3 | BD | MC38 FTY720 |
| | FOXP3 | PE-Cy7 | FJK-16s | eBiosciences | MC38 FTY720 |
| | H-2Kb - | PE | N/A | MBL | MC38 FTY720 |
| | Negative | | | | |
| | Tetramer | | | | |
| | (SIYRYYGL) | | | | |
| | H-2Kb MuLV | PE | N/A | MBL | MC38 FTY720 |
| | p15E Tetramer | | | | |
| | (KSPWFTTL) | | | | |
| | Ki67 | APC | 16A8 | Biolegend | MC38 FTY720 |
| | CD11b | BUV797 | M1/70 | BD | MC38 OXA |
| | CD11c | APCCY7 | N418 | eBiosciences | MC38 OXA |
| | CD155 | PE | 3F1 | BD | MC38 OXA |
| | CD206 | APC | MRS5D3 | eBiosciences | MC38 OXA |
| | CD45 | BV650 | 30-F11 | BD | MC38 OXA |
| | CD69 | APC-Cy7 | H1.2F3 | BD | MC38 OXA |
| | CD73 | EF450 | TY/11.8 | eBiosciences | MC38 OXA |
| | CD80 | BUV395 | 16-10A1 | BD | MC38 OXA |
| | DNAM | APC | 10E5 | eBiosciences | MC38 OXA |
| | F4/80 | AF488 | BMB | eBiosciences | MC38 OXA |
| | FOXP3 | Alexa Fluor | FJK-16s | eBiosciences | MC38 OXA |
| | | 700 | | | |
| | Granzyme B | PECy7 | NGZB | eBiosciences | MC38 OXA |
| | Ly6c | PECY7 | 1A8 | eBiosciences | MC38 OXA |
| | Ly6g | BV605 | HK1.1 | eBiosciences | MC38 OXA |
| | MHCII | AX700 | M5/114.15.2 | eBiosciences | MC38 OXA |
| | PD-L1 | BV711 | MIH4 | BD | MC38 OXA |
| | TIGIT | PE | GIGD7 | eBiosciences | MC38 OXA |
| Human | CCR7 | BV605 | 3D12 | BD | Human TIL |
| | CD103 | BV711 | Ber-ACT8 | BD | Human TIL |
| | CD14 | Ax700 | M5E2 | BD | Human TIL |
| | CD155 | BUV737 | SKII.4 | BD | Human TIL |
| | CD16 | BV605 | 3G8 | BD | Human TIL |
| | CD19 | PE | H1B19 | Thermo | Human TIL |
| | CD3 | APC-Cy7 | SK7 | Biolegend | Human TIL |
| | CD38 | BUV805 | HB7 | BD | Human TIL |
| | CD39 | BV786 | TU66 | BD | Human TIL |
| | CD45 | PE-Cy7 | HI30 | Biolegend | Human TIL |
| | CD45RA | BUV805 | HI100 | BD | Human TIL |
| | CD8 | BUV496 | RPA-T8 | BD | Human TIL |
| | CD95 | BUV395 | DX2 | BD | Human TIL |
| | DNAM | BUV395 | DX11 | BD | Human TIL |
| | DNAM | PE-Cy7 | 11A8.7.4 | Thermo | Human TIL |
| | FOXP3 | AF488 | PCH101 | Thermo | Human TIL |
| | GzmB | EF450 | N4TL33 | Thermo | Human TIL |
| | GzmK | AF488 | GM6C3 | Santa Cruz | Human TIL |
| | | | | Biotechnology | |
| | GzmK | AF647 | GM6C3 | Santa Cruz | Human TIL |
| | | | | Biotechnology | |
| | PD-1 | BV650 | EH12.1 | BD | Human TIL |
| | PD-L1 | BV421 | MIH1 | BD | Human TIL |
| | TCF1 | AF488 | C63D9 | CST | Human TIL |
| | TIGIT | PerCP-Cy5.5 | MBSA43 | Thermo | Human TIL |
| | TIM3 | BUV737 | 7D3 | BD | Human TIL |
| | CD56 | AF647 | B159 | BD | Human TIL |

TABLE 10
Human subject information

| Cancer type | Stage | Biopsy location |
|---|---|---|
| Esophageal | IV-A/Post-Tx | Esophagus |
| adenocarcinoma | | |
| Esophageal | Unknown/Post-Tx | Esophagus |
| adenocarcinoma | | |
| Gastro-esophageal | Unknown/Pre-Tx | Esophagus and cardia |
| Gastric | III-B/Pre-Tx | Middle and lower |
| | | third of stomach |
| NSCLC | III-A/Pre-Tx | Lower lobe - left lung |
| NSCLC | IV-A/Pre-Tx | Lung |
| NSCLC | II-A/Pre-Tx | Upper lobe - right lung |
| NSCLC | I-A/Pre-Tx | Right lung - upper lobe |
| NSCLC | III-A/Pre-Tx | Left lung - upper lobe |
| NSCLC | II-A/Post-Tx | left lung |
| | (resection only) | |
| NSCLC | III-A/Pre-Tx | Lung |
| NSCLC | III-A/Pre-Tx | Left lung - lower lobe |
| NSCLC | III-B/Pre-Tx | Left lung |
| NSCLC | I-A/Pre-Tx | Right lung - upper lobe |

Methods—Single-cell RNA-seq data processing and identification of Tₚₑₓ cells from scRNA-seq data. Count matrices for GSE99254 (Guo et al., 2018, Nature Medicine, 24(7), pp. 978-985) (Smart-Seq2) and GSE162498 (Gueguen et al., 2021, Science Immunology, 6(55), p. eabd57178) (IOX Genomics 5′ and 3′ assay) were downloaded from GEO and analyzed in the R Statistical Environment, v 4.2.2 (R Team, 2021). CD8⁺ T cells were filtered for cells expressing CD8A counts above and CD4 counts below a dataset-specific threshold (10 for Guo et al. and 1 for Gueguen et al., due to platform differences). The Gueguen et al. dataset consisted of samples sequenced using the 5′ and 3′ 10× Genomics assays. These samples were split by assay followed by separate library size adjustment using scran, v1.26.2 (Lun, Bach and Marioni, 2016). After normalization, the samples across both assays were integrated using the Seurat IntegrateData framework, v4.3.0 (Hao et al., 2021, Cell, 184(13), pp. 3573-3587.e29) with 2000 most variable features by “vst” as the search space for integration anchors. Following integration, the Seurat pipeline was used to normalize the data and clustree (v0.5.0) was used to identify the optimal resolution for clustering followed by UMAP dimensional reduction (McInnes, Healy and Melville, 2018, available at arxiv.org/abs/1802.03426v3). The Guo et al. dataset was processed using the described method minus integration, since the dataset consisted only of a single sequencing platform. Tₚₑₓ were identified by applying filters to single-cell clusters in the following order: medium to high expression of PD-1 (PDCD1), negative expression of TIM-3 (HAVCR2), and positive expression of granzyme K (GZMK). The local minima between modes were used as thresholds for filtering.

### Example 7

In this Example, the MC38 mouse model was leveraged to dissect mechanisms of anti-tumor efficacy afforded by combination therapy with anti-TIGIT and anti-PD-1.

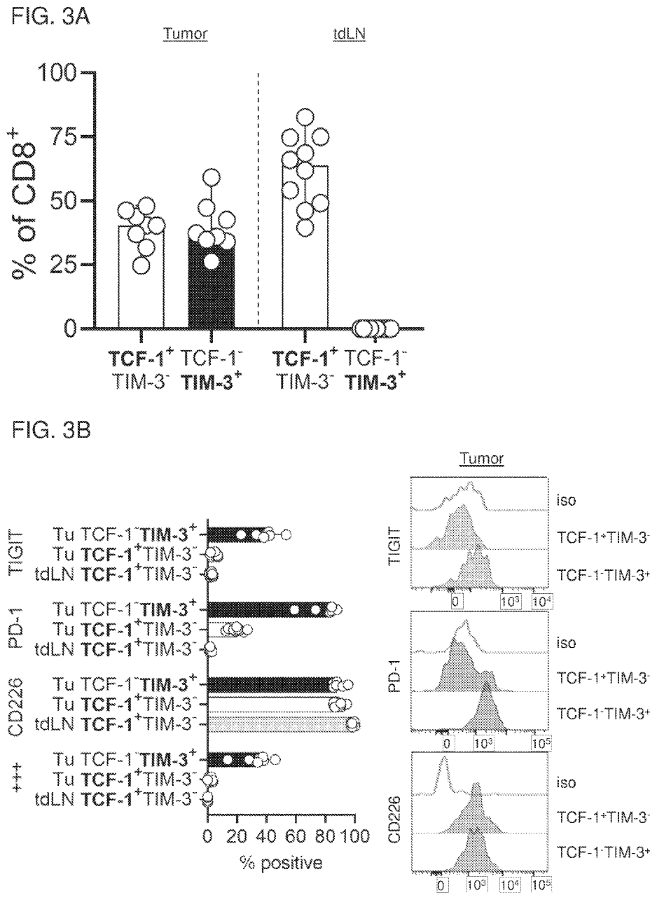

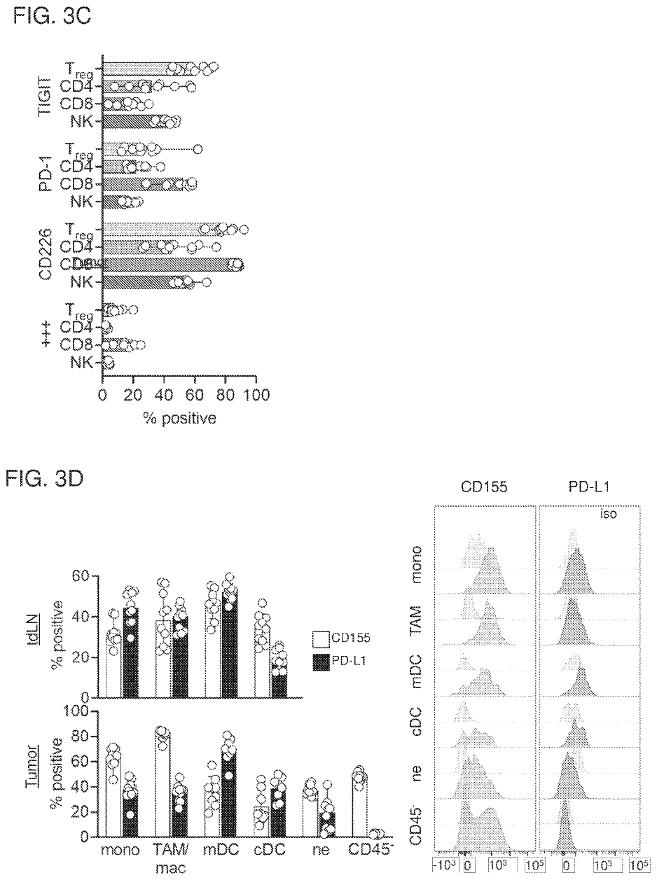

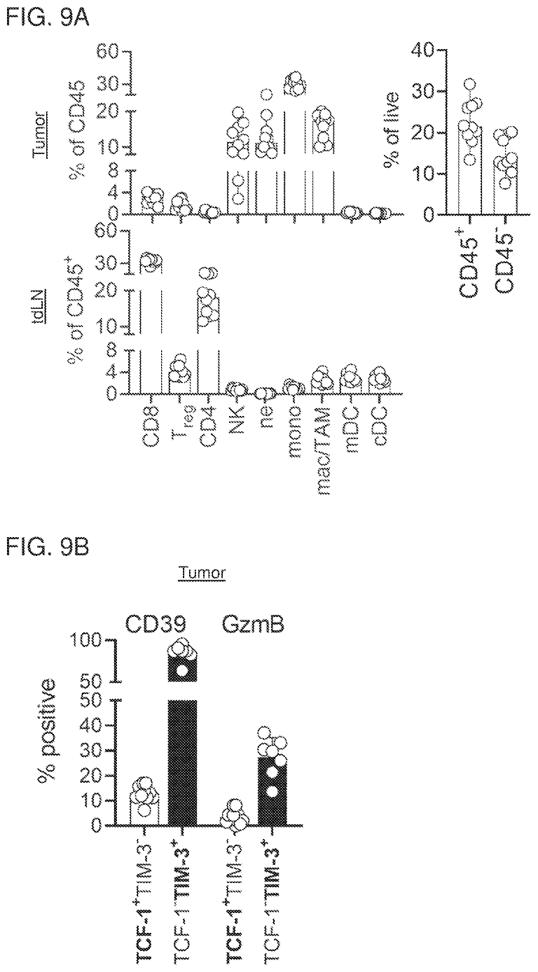

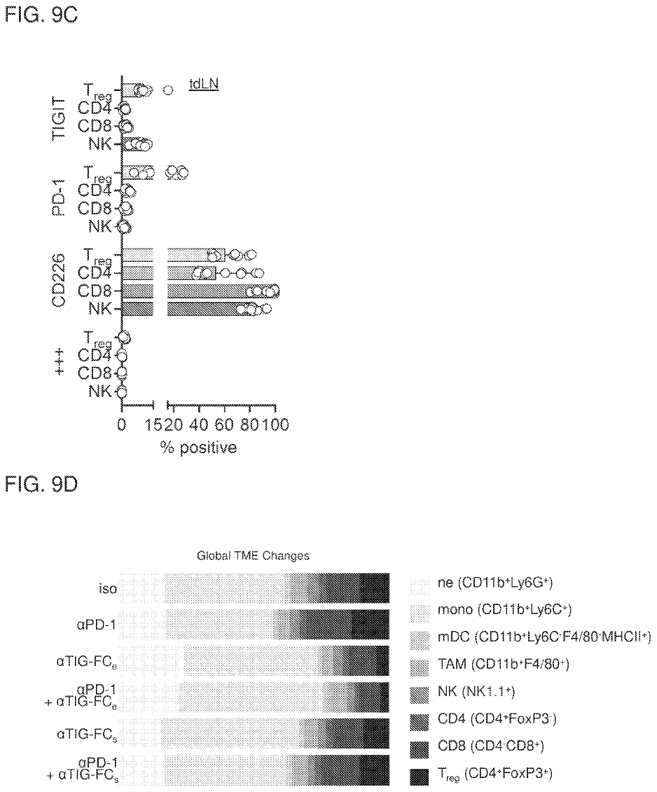

Tₑₓ cells expressing PD-1, TIGIT, and CD226 are present in mouse MC38 tumors. Tumors and tumor-draining lymph nodes (tdLN) were excised to evaluate cellular expression of relevant ligands and receptors. MC38 tumors were immune infiltrated, with high proportions of NK cells, tumor neutrophils and monocytes, and tumor-associated macrophages (TAMs) accompanying ~3-4% CD8⁺ T cells and, as expected, tdLNs contained a relatively high proportion of T cells and antigen-presenting populations (FIG. 7D, FIG. 7E, and FIG. 9A). Like human NSCLC and G-E TIL samples, Tₚₑₓ (TCF-1⁺TIM-3⁻) and Tₜₑₓ (TCF-1⁻TIM-3+) containing populations were detected (FIG. 3A and FIG. 7D-FIG. 7E). Tumor samples contained roughly equal proportions of these subsets, whereas TIM-3⁺ terminally differentiated cells were absent in the tdLN (FIG. 3A), consistent with lower levels of tumor antigen and the presence of co-stimulatory molecules in secondary lymphoid organs. While both populations expressed TIGIT and PD-1 in the tumor, TCF-1⁻TIM-3+Tₜₑₓ had higher frequencies of these receptors, co-expression of TIGIT and PD-1 with CD226, and expressed other molecules associated with effector function (GzmB) and terminal differentiation (CD39) (FIG. 3B and FIG. 9B). The tdLN TCF-1⁺TIM-3⁻ population expressed low levels of checkpoint receptors, suggesting this broad grouping likely contains a significant proportion of naïve and central memory cells expressing TCF-1, with a more modest Tₚₑₓ subset (FIG. 3B). Mirroring human NSCLC samples, tumoral T_{reg} had the most substantial TIGIT expression among broad T cell subsets (FIG. 3C). Except for T_{reg}, TIGIT and PD-1 expression was largely absent on tdLN T cell populations, whereas CD226 was expressed on a substantial proportion of cells in all populations interrogated (FIG. 3B, FIG. 3C, and FIG. 9C). In contrast to the human tumor-infiltrating lymphocyte (TIL) samples, a consistent abundance of NK cells in the TME permitted phenotypic assessment (FIG. 9A), and TIGIT, PD-1, and CD226 were expressed by tumoral NKs (FIG. 3C). Regarding cognate ligands, in addition to the CD45⁻ cancer and stromal cell population expressing PD-L1 in the tumor, various myeloid subsets contributed to PD-L1 and CD155 expression in both sites (FIG. 3D). Therefore, proteins relevant to PD-1 and TIGIT co-blockade were abundantly present in relevant compartments of the MC38 mouse model.

To elucidate differential effects of Fc-silent (Fcₛ) or Fc-enabled (Fcₑ) anti-TIGIT on tumor control, mouse TIGIT-specific antibodies engineered on a mouse IgG2a backbone with or without Fc silencing “LALA-PG” mutations (Lo et al., 2017, Journal of Biological Chemistry, 292(9), pp. 3900-3908) were able to enhance anti-PD-1-mediated tumor control in the MC38 model, consistent with previous reports (Johnston et al., 2014, Cancer Cell, 26(6), pp. 923-937; Dixon et al., 2018, The Journal of Immunology, 200(8), pp. 3000-3007). Notably, Fcₑ anti-TIGIT also demonstrated statistically significant single-agent activity, whereas Fcₛ did not. To understand this discrepancy, tumors were interrogated by flow cytometry three days after the first antibody treatment, allowing for detection of acute changes that precede dramatic alterations that correlate with tumor size. Cohorts treated with Fcₑ anti-TIGIT exhibited a significant loss in intratumoral T_{reg}, whereas this population was maintained in anti-PD-1 only and Fcₛ anti-TIGIT treated groups (FIG. 9D). Thus, both Fcₛ and Fcₑ anti-TIGIT enhance anti-tumor immunity in the presence of anti-PD-1, albeit by mechanisms that differentially shape the TME with respect to the T_{reg} population.

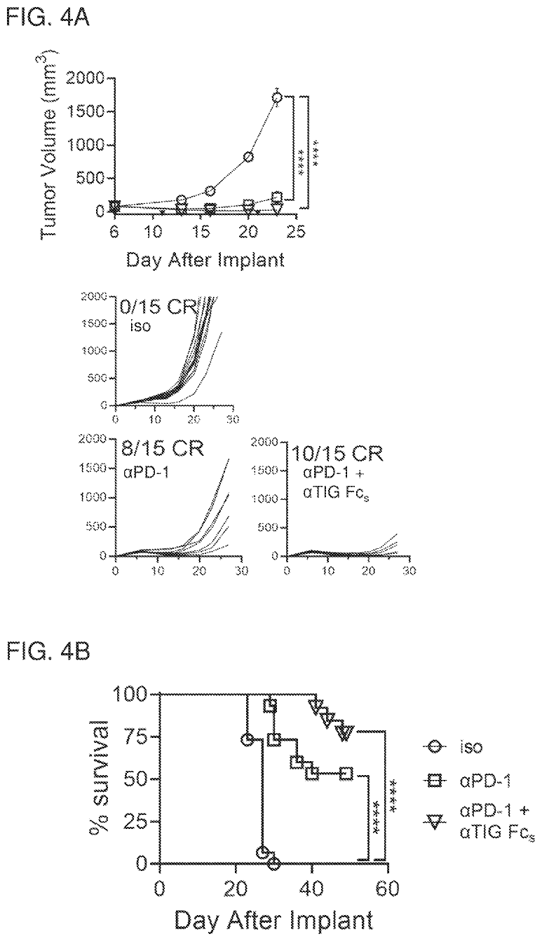

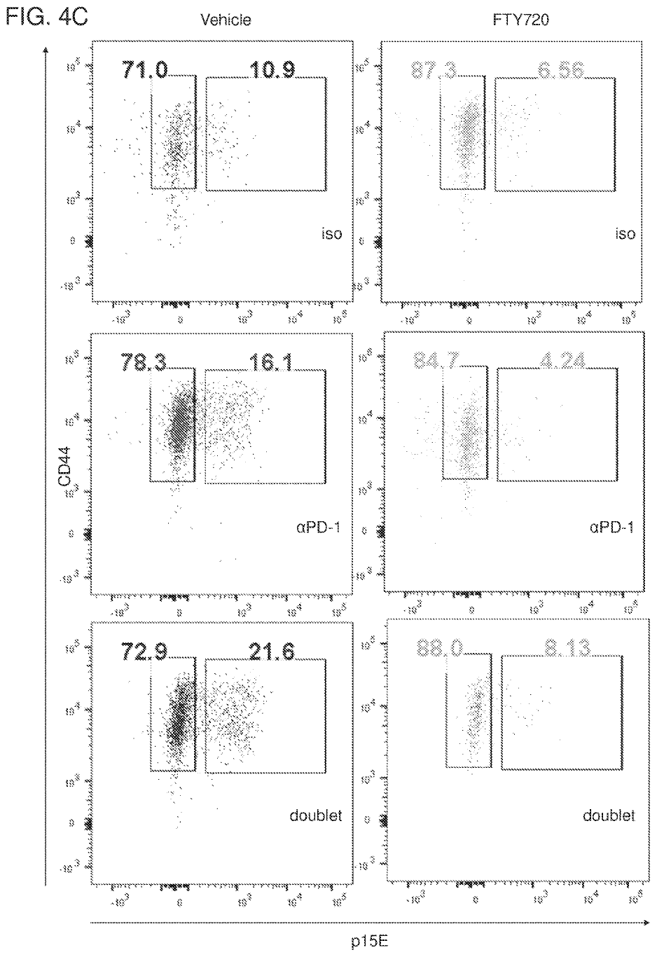

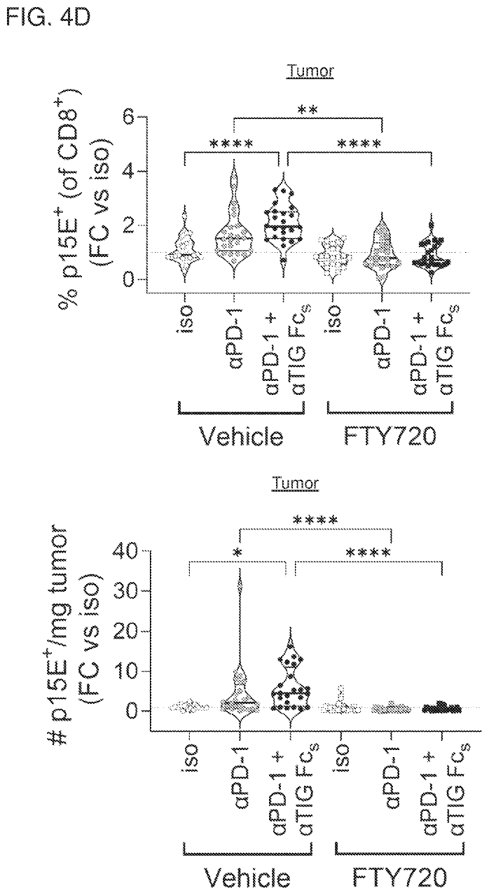

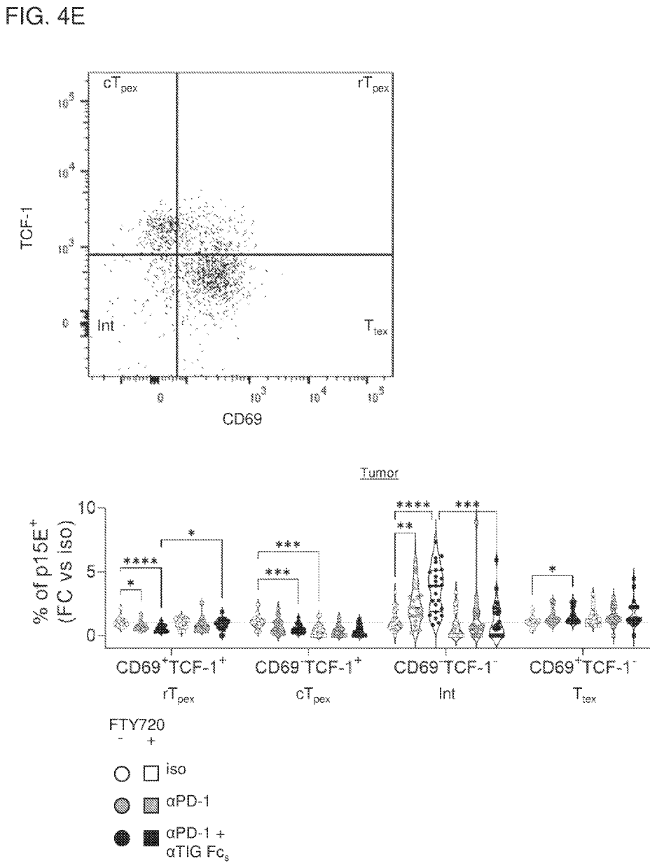

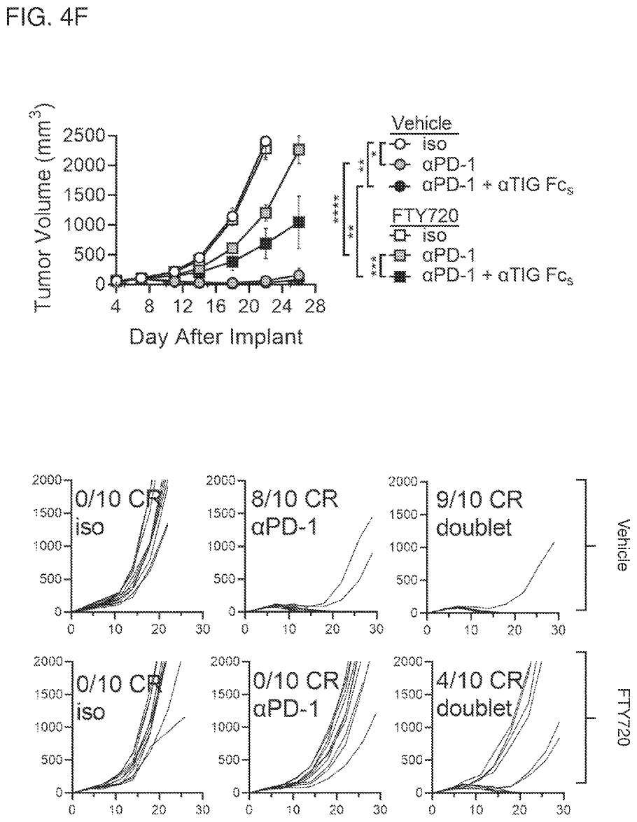

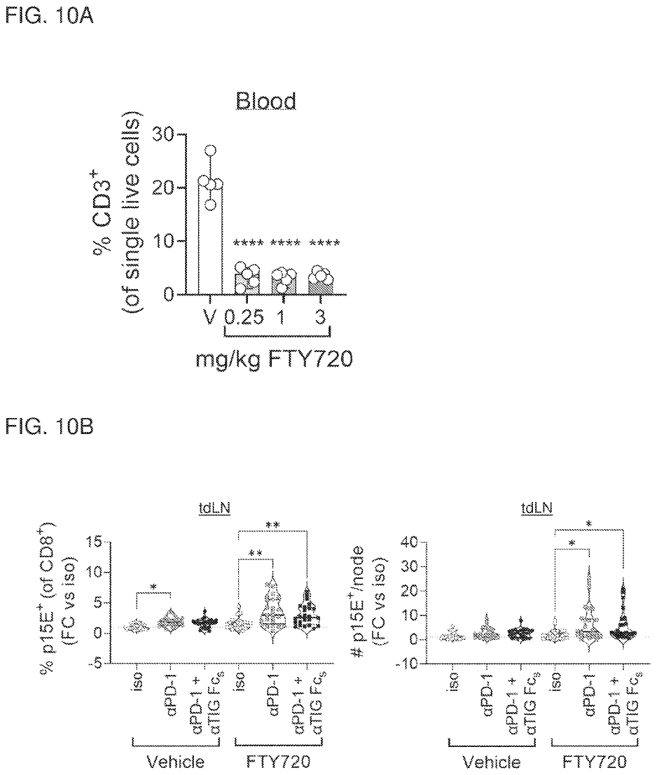

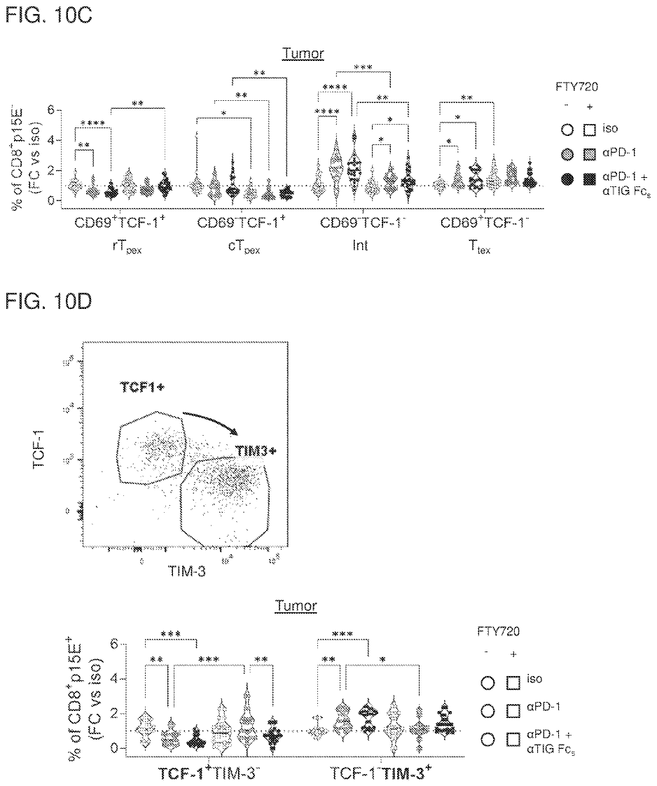

Anti-PD-1 in combination with Fcₛ anti-TIGIT enhances endogenous tumor-specific T cell responses in a tdLN-dependent manner. To examine cellular mechanisms by which the Fcₛ antibody promoted anti-tumor immunity, tumors and tdLNs were evaluated for changes in CD8⁺ Tₑ populations specific for the endogenous murine retroviral envelope epitope p15E (KSPWFTTL) expressed by MC38 cells (Ye et al., 2020, OncoImmunology, 9(1)). Additionally, cohorts of mice receiving therapeutic antibodies also received daily oral gavages of S1PR1 agonist, FTY720, to differentiate the effects of T cells present within the tumor from T cells arriving after recirculation from the tdLN (Chiba, 2005, Pharmacology and Therapeutics. Pergamon, pp. 308-319) (FIG. 10A). After three days of I-O therapy, CD8⁺p15E⁺ tumor-specific T cells in anti-PD-1 plus Fcₛ anti-TIGIT (hereinafter in this Example referred to as “doublet”) treated cohorts increased numerically relative to single-agent anti-PD-1; moreover, doublet treated cohorts had statistically significant increases in frequency and number relative to vehicle control cohorts, whereas the anti-PD-1 single agent treated group did not (FIG. 4C and FIG. 4D). Notably, these increases were lost when T cell egress from the tdLN was blocked with FTY720, indicating a role for secondary lymphoid organs in doublet activity (FIG. 4C and FIG. 4D). Corresponding accumulations in p15E⁺ cells were detected in the tdLNs of I-O treated animals given FYT720 (FIG. 10B).

The phenotype and differentiation status of p15E⁺ cells in doublet-treated mice were evaluated next. Tₑₓ sub-populations were gated based on expression of tissue residence/activation marker CD69 and transcription factor TCF-1 (Miller et al., 2019, Nature Immunology, 20(3), pp. 326-336; Beltra et al., 2020), resulting in four subsets that contain functionally distinct populations: TCF-1⁺CD69⁺ resident Tₚₑₓ, TCF-1⁺CD69 circulating Tₚₑₓ, TCF-1⁻CD69⁺ intermediate effector, and TCF-1⁻CD69⁻ Tₜₑₓ. Consistent with the demonstration of Tₚₑₓ activation and differentiation with anti-PD-1 (Miller et al., 2019; Siddiqui et al., 2019; Beltra et al., 2020), doublet treatment triggered statistically significant decreases in TCF-1⁺CD69⁺ and TCF-1⁺CD69⁻ Tₚₑₓ populations, and reciprocal increases in the TCF-1⁻ CD69⁻ intermediate effector and TCF-1⁻CD69⁺ terminally differentiated p15E⁺ subsets (FIG. 4E). Notably, doublet treated animals had more differentiated changes in these populations relative to anti-PD-1 alone. Similar trends were observed in the bulk CD44⁺CD8⁺p15E⁻ population (representing bystander or p15E⁻ tumor-reactive T cells) (FIG. 10C) and when using the TCF-1/TIM-3 gating strategy (FIG. 10D). All changes were largely diminished when T cell recirculation was blocked (FIG. 4E, FIG. 10C, and FIG. 10D). Likewise, tumor control afforded by anti-PD-1 alone or doublet treatment was also compromised in the presence of FTY720 (FIG. 4F), indicating that T cell trafficking from the tdLN was required for maximal anti-tumor immunity in anti-PD-1 and doublet treated mice. Of note, tumor control by the doublet treatment was only partially abolished in the presence of FTY720, with 40% of the cohort (4/10 mice) still exhibiting complete regressions (CR) and statistically significant tumor control relative to the anti-PD-1 monotherapy mice receiving FTY720 (FIG. 4F).

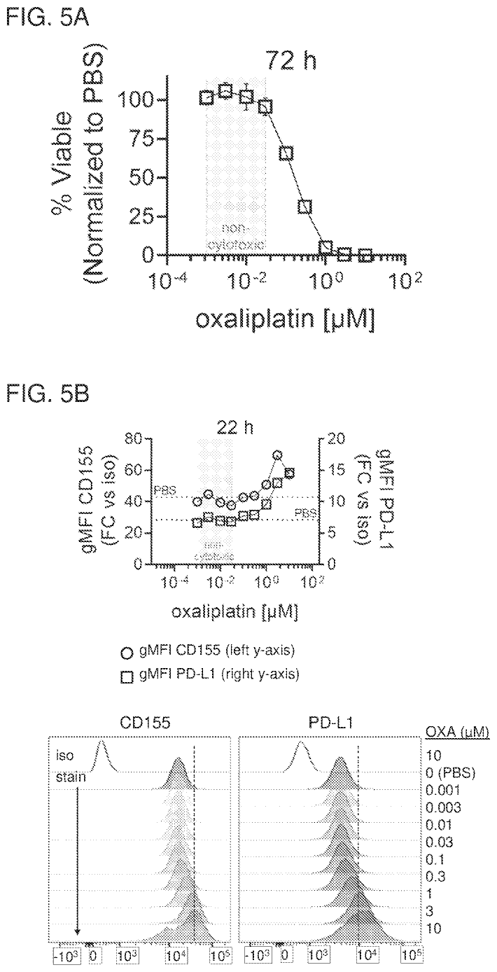

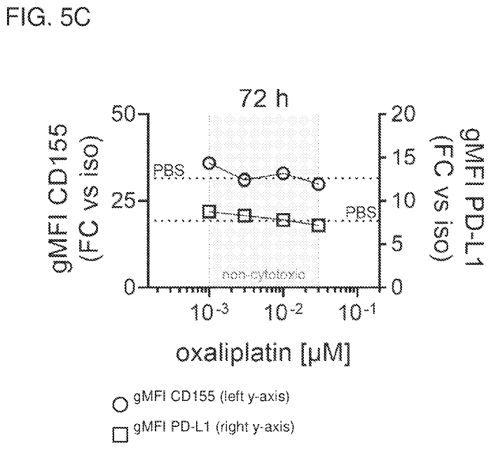

It was postulated that combination of TIGIT and PD-1 blocking antibodies with chemotherapeutic agents that have been reported to incite immunogenic cell death, like oxaliplatin (OXA) (Galluzzi et al., 2020), would lead to enhanced tumor control. To isolate the effect of OXA upon MC38 cancer cells as it relates to TIGIT and PD-1 biology, in vitro cultures were assessed for cell death and ligand expression. By 72 hours (h), nearly 100% cancer cell death was observed using 1 μM OXA (FIG. 5A). At the 22 h time point, dying cells (defined as cells treated with ≥1 μM OXA with an intact cell membrane as assessed by live-dead staining) increased expression of PD-L1 and CD155 ligands (FIG. 5B). Of note, cells treated with non-lethal concentrations of OXA (<0.03 μM) for up to 72 h did not exhibit increases in PD-L1 or CD155 (FIG. 5A, FIG. 5B, and FIG. 5C).

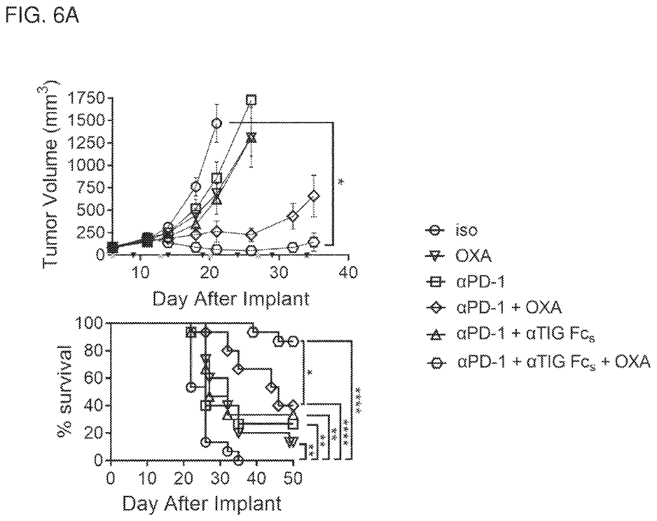

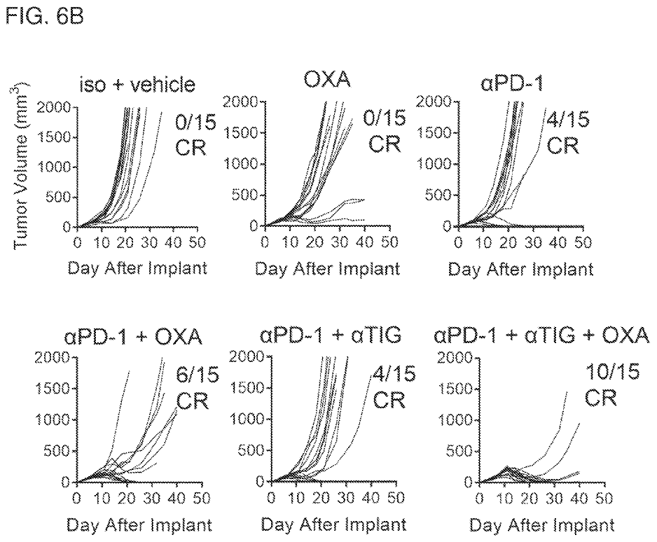

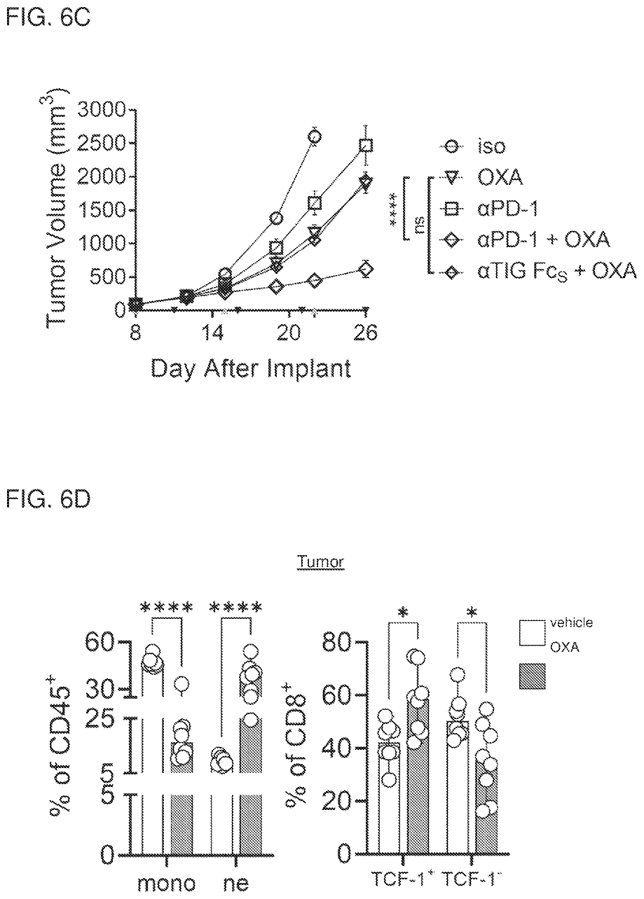

To prime the tumor microenvironment (TME) for optimal immuno-oncology (1-0) efficacy, mice bearing MC38 tumors were treated with OXA three days prior to receiving anti-PD-1 plus Fes anti-TIGIT (herein referred to as “doublet”) regimen. Because of the tumor size and/or composition at the time of I-O therapy, doublet treatment had only a modest effect in delaying tumor growth, similar to the effect of chemotherapy alone (FIG. 6A and FIG. 6B). Fes anti-TIGIT alone did not enhance the effect of OXA (FIG. 6C). However, combining OXA with anti-PD-1 increased survival relative to either monotherapy and the addition of Fes anti-TIGIT significantly enhanced survival, with 67% of the triplet cohort achieving a complete response (FIG. 6A and FIG. 6B).

Given the significant anti-tumor efficacy seen in the triplet combination, how OXA exposure modulated the composition of the TME was assessed. Tumors and tdLNs were isolated three days after the first exposure and interrogated by flow cytometry for changes in lineage frequencies. Robust and reproducible changes were only observed in the tumor and included decreases in intratumoral monocytes accompanied by an increase in tumor neutrophils (FIG. 6D, FIG. 7D, and FIG. 7E). Within the tumoral CD8⁺ T cell compartment, clear changes were not observed when analyzing granular subsets (using TCF-1/TIM-3 or TCF-1/CD69 gating strategies); however, an increase in TCF-1 positivity overall was noted (FIG. 6D). Together, these data are suggestive of a pro-inflammatory shift by OXA in the TME that can be capitalized upon with PD-1 and TIGIT co-blockade.

Materials—For mouse studies, murine antibodies that target mouse PD-1 and TIGIT receptors were supplied by Arcus Biosciences. Purified anti-PD-1 (clone RMP1-14), anti-TIGIT (clone 10A7), and isotype control (clone MOPC-173) antibodies were generated on a mouse IgG2a backbone. Antibodies of clone 10A7 used sequences disclosed in patent publication WO2017/053748. L234A, L235A, and P329G (Eu numbering) mutations in the heavy chain were used to render anti-PD-1 and anti-TIGIT Fcₛ mouse-specific antibodies Fc non-functional. Antibodies supplied by Arcus Biosciences were expressed in CHO or HEK293 cell lines. Bacterial endotoxin levels were measured using the limulus amebocyte lysate (LAL) test and were <1 EU/mg. Mouse antibodies used for flow cytometry are summarized in Table 8. Oxaliplatin was purchased from Sigma-Aldrich and dissolved in PBS (in vitro) and saline (in vivo). FTY720 was purchased from Sigma-Aldrich was dissolved in water. Murine MC38 cells were obtained from Kerafast and cultured in RPMI 1640 medium containing 10% FBS, 100 U/mL penicillin/streptomycin, and 1× GlutaMAX (all Gibco). Cell lines were authenticated using short tandem repeat DNA profiling and tested for pathogen contamination including Mycoplasma spp. (STAT-Myco and IMPACT II testing) via RT-PCR (IDEXX Bioresearch). All cell lines were maintained at 37° C., 21% 02, 5% CO₂.

Methods—Mouse tumor-infiltrating lymphocyte and lymph node phenotyping. MC38 tumors were minced with scissors and dissociated with tumor digestion buffer [RPMI 1640 supplemented with 20% FBS, 0.25 mg/mL Collagenase D (Roche), and 100 KUnits/mL Type IV DNase I (Sigma-Aldrich)]. Single-cell tumor suspensions were passed through a 70 μm cell strainer and washed. Inguinal lymph nodes were collected from the same side as the tumor (tumor-draining lymph node, tdLN), minced, passed through a 70 μm cell strainer and washed. A total of 1×10⁶ tumor or lymph node cells per well were incubated at room temperature (RT) with live-dead NIR (Invitrogen) and anti-mouse CD16/CD32 Fc block (BD Biosciences). For p15E tetramer staining, cells were washed and incubated with tetramer at RT. Cells were stained for surface and intracellular markers and analyzed by flow cytometry as indicated above.

In vivo experiments were performed at Arcus Biosciences in accordance with federal, state, and institutional guidelines and were approved by Arcus Biosciences' Institutional Animal Care and Use Committee. Female 6-8 week old C57BL/6J mice were purchased from Charles River Laboratories. Mice were injected subcutaneously on the right flank with 1×10⁶ MC38 cells in 100 μL PBS. Once tumors were established (sized indicated in figure legends), mice were randomized into groups based on tumor size distribution. Antibody and OXA injections were given intraperitoneally and FTY720 was administered by oral gavage at concentrations and frequencies indicated in the figure legends. When given together, anti-PD-1 and anti-TIGIT were co-formulated for most studies. Appropriate antibody isotype control was given to match the antibody concentration and injection of therapeutic antibodies. The dosing regimens for anti-PD-1 and anti-TIGIT were determined via receptor occupancy (RO) assays using commercially available a competitive anti-TIGIT antibody (clone GIGD7), a non-competitive PD-1 antibody (clone J43) as well as direct detection of both anti-TIGIT and anti-PD-1 via secondary antibody (Jackson Immunoresearch anti-mouse IgG, Fcγ subclass 2a specific). The dosing regimen for FTY720 was determined in naïve C57BL/6 females by examining lymphocyte frequencies and numbers in the blood 24 h after dosing. Tumor volume was monitored with digital calipers and volume was calculated using the formula: (Length×Width²)/2. For survival, a predetermined tumor volume of 2000 mm³ was used as the endpoint.

In vitro oxaliplatin treatment of MC38 cells. For flow cytometry, MC38 cells were plated in supplemented RPMI medium at 1×10⁵ cells/well in a 12-well tissue culture plate. For CellTiter-Glo (Promega) analysis, cells were plated in supplemented RPMI medium at 5×10³ cells/well in a 96-well white flat-bottom tissue culture plate. PBS or oxaliplatin was added at concentrations ranging from 1-10,000 nM and incubated at 37° C. as indicated in figure legends, and viability was assessed according to the manufacturer's instructions. Data were normalized to PBS treated cells. For flow cytometry, cells were harvested using a cell scraper, and transferred into a 96-well round-bottom plate for staining. Cells were washed, incubated with live-dead aqua and anti-mouse CD16/CD32 Fc block at RT followed by cell surface staining with anti-mouse PD-L1 BV711 (clone MIH4, BD Biosciences) and anti-mouse CD155 BUV395 (clone 3F1, BD Biosciences) at 4° C. Cells were washed, fixed with IC fixation buffer (Invitrogen) at RT, washed again, and resuspended in PBS for flow cytometry.

Statistical analysis. Statistical analyses were completed using Prism (GraphPad) or R. Differences between the control and treated groups were analyzed by either Dunnett test, Tukey test, or t test as stated in figure legends. P values less than or equal to 0.05 were considered significant. For survival experiments, Kaplan-Meier survival plots were generated and three comparisons between groups were conducted using a family-wise significance level of 5%.

### Example 8

This example retrospectively evaluated the predictive value of CD155 expression for a clinical outcome using real-world data. In order to assess associations to clinical outcome using real-world data, real-world progression free survival analyses were performed. Real-world progression free survival (rwPFS) is defined in this study as the time from treatment initiation until the first occurrence of progressive disease (PD) or death up to the end date of the same line of therapy. A change in therapy was also counted as a progression event, even where none was directly captured. Otherwise, patients were censored at the last known follow up time. rwPFS is right censored at 36 months. The analysis was performed using de-identified health records of first line (1L) metastatic NSCLC patients treated with chemotherapy only, treated with pembrolizumab monotherapy, or treated with chemotherapy and pembrolizumab. A filter was applied to select for records where biopsies were obtained ≤12 months prior to the first dose of treatment. Records were excluded if the patient had EGF or ALK alterations.

The health records for each patient included information such as sex, race, smoking status, biopsy site, stage at time of diagnosis, subtype of lung cancer (adenocarcinoma or squamous cell carcinoma), tumor purity, PD-L1 expression, treatment (chemotherapy, pembrolizumab, or chemotherapy+pembrolizumab), clinical outcome, etc., as well as gene expression data for a number of targets (e.g., TIGIT, CD155, CD73). Chemotherapy for adenocarcinomas was cisplatin or carboplatin+pemetrexed. Chemotherapy for squamous cell carcinomas was either carboplatin and paclitaxel or nab-paclitaxel. Gene expression was quantified from RNA-seq data as transcript per million (tpm). Kaplan-Meier models were performed by gene and according to the following breakdowns: NSCLC subtype (all; adenocarcinoma only; squamous cell carcinoma only), PD-L1 expression (all; <1% TPS; 1-49% TPS; ≥50% TPS), and treatment (chemotherapy only, pembrolizumab only, chemotherapy and pembrolizumab). Results presented as Kaplan-Meier (KM) plots splitting high and low gene expressors into different groups, whereby high gene expression was defined as expression equal to or greater than a cut-off value determined by one of several threshold methodologies (e.g., median, 1^{st} tertile (1^{st} third vs last two thirds), 2^{nd} tertile (two thirds vs. last third) and MaxStat (an outcome-oriented method providing a value of a cutpoint that corresponds to the most significant relation with outcome). Cox Proportional-Hazards tests were used to assess the effect of gene expression as a continuous variable, and hazard ratios were provided with each KM plot using the low gene expressor group as a reference.

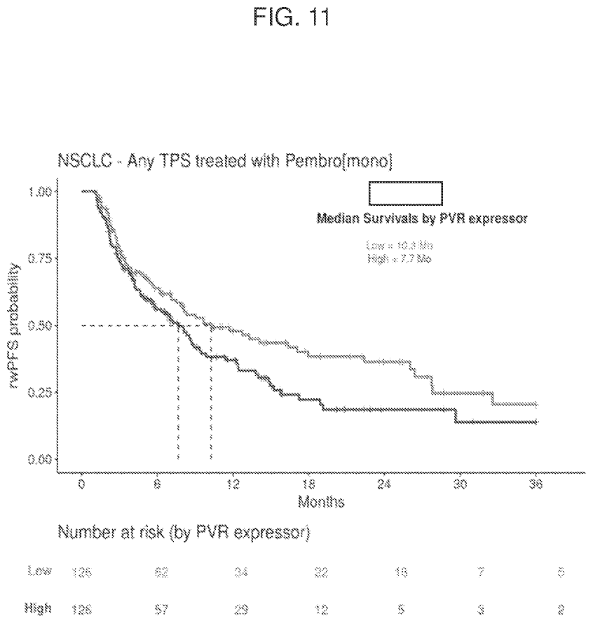

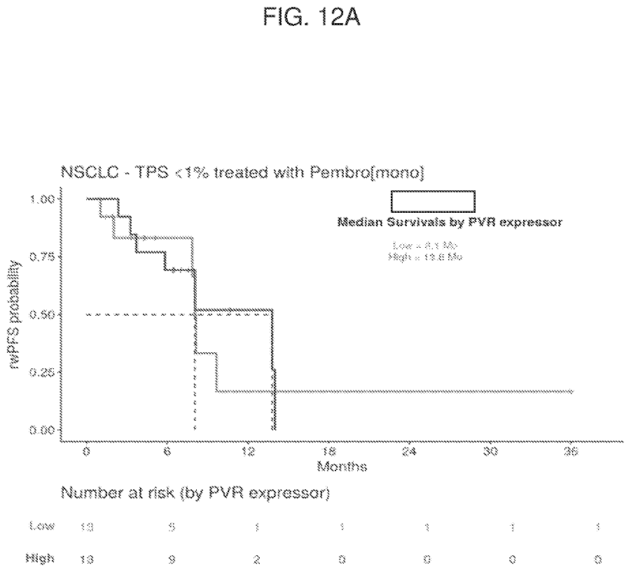

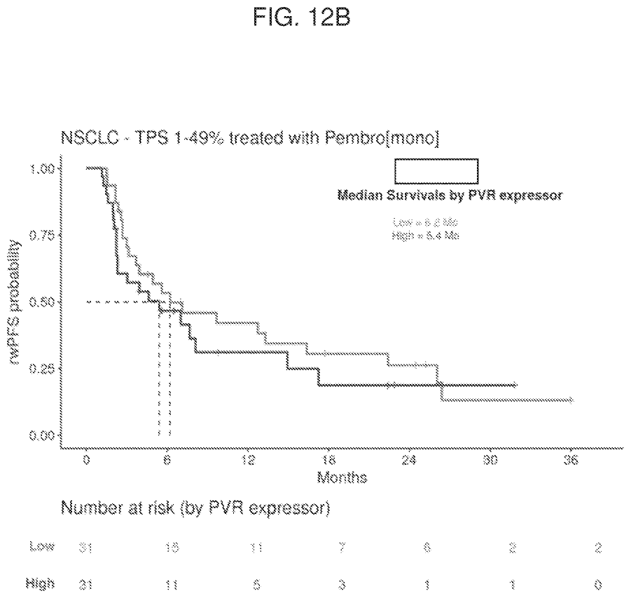

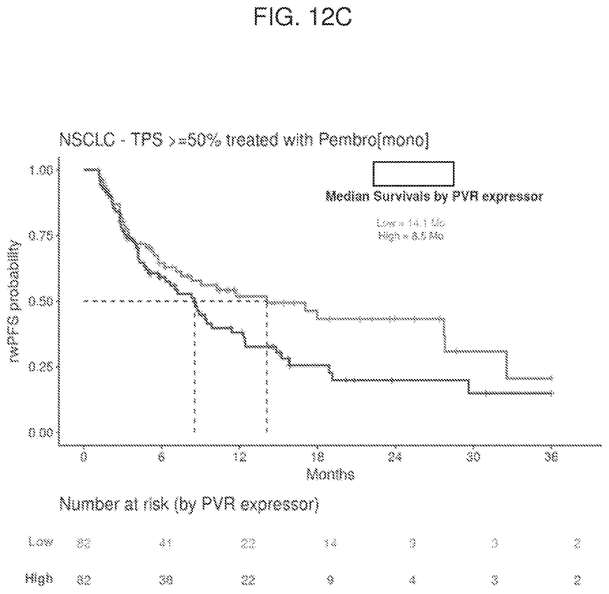

FIG. 11 shows a KM plot for the pembrolizumab monotherapy treatment group by CD155 expression using median CD155 expression as the cut-off value. These data show 1L metastatic NSCLC patients least likely to benefit from pembrolizumab monotherapy had high CD155 gene expression. A cohort analysis based on PD-L1 expression is shown in FIG. 12A-C (FIG. 12A: TPS <1; FIG. 12B: TPS=1-49; FIG. 12C: TPS ≥50). This analysis shows that high CD155 expression is predictive for clinical response to pembrolizumab in PD-L1 positive patients (TPS ≥1). Additional data are shown in Table 11.

TABLE 10

| | | | CD155 High | | CD155 Low | |
|---|---|---|---|---|---|---|
| Treatment: | Threshold | Threshold | Median | | Median | |
| PD-L1 TPS | method | value | OS (mo) | (n) | OS (mo) | n |
| Pembro: All | q33 | 4.77 | 8.07 | 170 | 13.3 | 82 |
| Pembro: <1% | q33 | 4.63 | 8.1 | 17 | 8.1 | 9 |
| Pembro: 1-49% | q33 | 4.66 | 4.63 | 41 | 9.67 | 21 |
| Pembro: ≥50% | q33 | 4.89 | 8.63 | 110 | 17.1 | 54 |
| Pembro: All | Median | 5.04 | 7.67 | 126 | 10.3 | 126 |
| Pembro: <1% | Median | 4.85 | 13.8 | 13 | 8.1 | 13 |
| Pembro: 1-49% | Median | 4.86 | 5.4 | 31 | 6.2 | 31 |
| Pembro: ≥50% | Median | 5.1 | 8.53 | 82 | 14.1 | 82 |
| Pembro: All | q67 | 5.35 | 7.03 | 82 | 9.67 | 170 |
| Pembro: <1% | q67 | 5.27 | 13.8 | 9 | 8.1 | 17 |
| Pembro: 1-49% | q67 | 5.2 | 3.93 | 20 | 7.1 | 42 |
| Pembro: ≥50% | q67 | 5.45 | 7.23 | 55 | 11.7 | 109 |
| Combo: All | q33 | 4.72 | 6.77 | 559 | 7.37 | 273 |
| Combo: <1% | q33 | 4.63 | 6.3 | 247 | 6.43 | 122 |
| Combo: 1-49% | q33 | 4.69 | 6.67 | 208 | 7.6 | 102 |
| Combo: ≥50% | q33 | 4.95 | 9.57 | 103 | 8.97 | 50 |
| Combo: All | Median | 4.97 | 6.93 | 412 | 6.97 | 420 |
| Combo: <1% | Median | 4.92 | 6.4 | 185 | 6.33 | 184 |
| Combo: 1-49% | Median | 4.9 | 6.9 | 156 | 6.9 | 154 |
| Combo: ≥50% | Median | 5.25 | 9.77 | 76 | 8.97 | 77 |
| Combo: All | q67 | 5.23 | 7.03 | 274 | 6.93 | 558 |
| Combo: <1% | q67 | 5.17 | 6.4 | 122 | 6.37 | 247 |
| 1-49% | q67 | 5.15 | 6.67 | 103 | 7.07 | 207 |
| >=50% | q67 | 5.52 | 11.7 | 50 | 8.8 | 103 |

### Example 9

Soluble biomarkers were retrospectively analyzed in baseline and on-treatment peripheral blood derived samples obtained from a subset of subjects enrolled in the clinical trial of Example 2. Baseline samples were obtained on day 1 of cycle 1 (CID1), prior to administration of any study drugs. On-treatment samples were obtained on day 1 of cycle 2 (C2D1), prior to administration of study drugs on that day.

Blood collected in serum separator tubes was allowed to clot for 30-60 minutes before centrifugation at 1300 g for 10 minutes to separate the serum. Serum was isolated and stored at −80° C. until cytokine analysis. Serum cytokines were quantified in multiplex assays using the Meso Scale Discovery platform following the manufacturer's protocol. Briefly, plates were coated with biotinylated capture antibodies specific to the analyte of interest bound to linkers specific to unique spots in each well of a 96-well plate. Serum samples, assessed in duplicate, were diluted 8-fold to quantify CXCL9, CXCL10, CXCL11 and IL-18 in a U-plex assay, diluted 10-fold to quantify IL-12B in a U-plex assay, and diluted 5-fold to quantify IFNγ, TNFα and IL-6 in the S-plex assay. Samples were incubated on linker/capture antibody-coated plates, followed by detection with a sulfo-tag-labelled antibody for the U-plex assays and a TURBO-tag-labelled antibody for the S-plex assay. For the S-plex assay, additional steps incorporated in the manufacturer's protocol to amplify signal with enhance solution and detection solution were followed. Following addition of read buffer, the electrochemiluminescent signal was measured on a MESO Sector S 600 instrument. Between each step of the protocol plates were washed with 0.05% Tween-PBS. A standard curve was generated for each plate using a 4-parameter logistic fit with 1/y² weighting. Cytokine concentrations in samples were interpolated using the standard curve, allowing quantification of the analyte of interest. Fold change from baseline was calculated as a ratio of the cytokine concentration at C2D1 over the cytokine concentration at baseline. A linear mixed model performed on log transformed raw cytokine values with baseline values as covariates assessed the difference in cytokine levels over time and between treatment groups.

CXCL9, CXCL10, CXCL11, IL-12B, IL-18, IL-6, IFNγ, TNFα levels measured at baseline and C2D1 are summarized in Table 11.

TABLE 11

| | Zimberelimab | Domvanalimab |
|---|---|---|
| | Monotherapy | Combination |
| | (Zim Mono; | (Dom Combo; |
| | Arm 1) | Arm 2 + 3) |
| CXCL9 (pg/mL) | | |
| Baseline, median | 277 | 280 |
| C2D1, median | 621 | 455 |
| Fold change, median | 2.4 | 1.7 |
| CXCL10 (pg/mL) | | |
| Baseline, median | 448 | 437 |
| C2D1, median | 639 | 638 |
| Fold change, median | 1.7 | 1.4 |
| CXCL11 (pg/mL) | | |
| Baseline, median | 105 | 104 |
| C2D1, median | 172 | 152 |
| Fold change, median | 1.6 | 1.3 |
| IL-12B (pg/mL) | | |
| Baseline, median | 220 | 221 |
| C2D1, median | 284 | 247 |
| Fold change, median | 1.5 | 1.1 |
| IL-18 (pg/mL) | | |
| Baseline, median | 737 | 777 |
| C2D1, median | 779 | 907 |
| Fold change, median | 1.2 | 1.1 |
| IL-6 (fg/mL) | | |
| Baseline, median | 15740 | 15205 |
| C2D1, median | 14385 | 10480 |
| Fold change, median | 1.0 | 0.7 |
| IFNγ (fg/mL) | | |
| Baseline, median | 711 | 843 |
| C2D1, median | 1100 | 1005 |
| Fold change, median | 1.6 | 1.2 |
| TNFα (fg/mL) | | |
| Baseline, median | 1174 | 1212 |
| C2D1, median | 1336 | 1263 |
| Fold change, median | 1.2 | 1.1 |

Arm 1: zimberelimab monotherapy; Arm 2: zimberelimab + domvanalimab combination therapy; Arm 3: zimberelimab + domvanalimab + etrumadenant combination therapy

Kaplan-Meier survival plots using progression-free survival data and cytokine concentrations were generated in the Jmp software, and hazard ratios were calculated between patient groups of interest. Comparisons were made for each analyte between zimberelimab monotherapy and domvanalimab combination groups both at baseline and using the fold change at C2D1 using median values to stratify patients in to low and high cytokine groups. Stratifying subjects by baseline cytokine expression (e.g., CXCL9, CXCL10, CXCL11, LL-6, TNFα) around the median value (high vs. low) enriched for clinical benefit with the addition of domvanalimab. See Table 12. Following treatment, peripheral cytokine levels changed as shown in Table 11 and Table 13. Baseline reference values derived from the biomarker evaluable population (BEP) are as follows (high is ≥reference value, and low is <reference value): CXCL9: 278.7 pg/mL; CXCL10: 445.4 pg/mL; CXCL11:104.2 pg/mL; L-6: 15550 fg/mL; TNFα: 1194 fg/mL. Induction reference values derived from the BEP are as follows (high is ≥reference value, and low is <reference value): CXCL9: 1.76 fold change; CXCL10: 1.52 fold change; IL6-12: 1.14 fold change IL-18: 1.1 fold change; IFNγ: 1.23 fold change.

TABLE 12
Median PFS in biomarker selected subsets
of the BEP (baseline samples)

| | | PFS, mo | HR vs. Zim Mono |
|---|---|---|---|
| Biomarker, Treatment Arm | n | (95% CI) | (95% CI) |
| BEP, Zim Mono | 49 | 5.6 | — |
| | | [1.84, 9.66] | |
| BEP, Dom Combo | 95 | 9.7 | 0.71 |
| | | [5.49, 13.18] | [0.46, 1.1] |
| CXCL9 Low at Baseline, | 25 | 7.6 | — |
| Zim Mono | | [1.68, 11.73] | |
| CXCL9 Low at Baseline, | 47 | 7.6 | 0.84 |
| Dom Combo | | [4.01, 14.62] | [0.45, 1.6] |
| CXCL9 High at Baseline, | 24 | 4 | |
| Zim Mono | | [1.48, 9.66] | |
| CXCL9 High at Baseline, | 48 | 10.9 | 0.58 |
| Dom Combo | | [4.07, NE] | [0.31, 1.08] |
| CXCL10 Low at Baseline, | 24 | 7.6 | — |
| Zim Mono | | [1.68, 9.69] | |
| CXCL10 Low at Baseline, | 48 | 7.7 | 0.82 |
| Dom Combo | | [4.83, 12.98] | [0.44, 1.54] |
| CXCL10 High at Baseline, | 25 | 5.4 | — |
| Zim Mono | | [1.48, 11.73] | |
| CXCL10 High at Baseline, | 47 | 12.0 | 0.59 |
| Dom Combo | | [3.8, NE] | [0.32, 1.11] |
| CXCL11 Low at Baseline, | 24 | 5.6 | — |
| Zim Mono | | [1.84, 9.56] | |
| CXCL11 Low at Baseline, | 48 | 10.9 | 0.59 |
| Dom Combo | | [6.7, 25.4] | [0.31, 1.09] |
| CXCL11 High at Baseline, | 25 | 4.9 | — |
| Zim Mono | | [1.5, 14.8] | |
| CXCL11 High at Baseline, | 47 | 6.3 | 0.84 |
| Dom Combo | | [3.0, 17.7] | [0.45, 1.57] |
| IL-6 Low at Baseline, | 24 | 9.6 | — |
| Zim Mono | | [5.36, NE] | |
| IL-6 Low at Baseline, | 48 | 13.9 | 0.79 |
| Dom Combo | | [7.69, NE] | [0.41, 1.55] |
| IL-6 High at Baseline, | 25 | 1.8 | — |
| Zim Mono | | [1.38, 5.59] | |
| IL-6 High at Baseline, | 48 | 5.5 | 0.53 |
| Dom Combo | | [3.25, 12.98] | [0.29, 0.97] |
| TNFα Low at Baseline, | 25 | 5.7 | — |
| Zim Mono | | [2.69, 11.73] | |
| TNFα Low at Baseline, | 48 | 13.9 | 0.57 |
| Dom Combo | | [7.69, 25.4] | [0.30, 1.07] |
| TNFα High at Baseline, | 24 | 3.5 | — |
| Zim Mono | | [1.38, 14.82] | |
| TNFα High at Baseline, | 49 | 4.9 | 0.85 |
| Dom Combo | | [3.15, 9.66] | [0.46, 1.57] |

BEP: biomarker evaluable population for cytokine analysis, individual numbers may vary; HR: Hazard Ratio, PFS: progression free survival; NE: not evaluable; Zim mono: zimberelimab monotherapy (See Table 11); Dom combo: domvanalimab combination (See Table 11)

TABLE 13
Median PFS in biomarker selected subsets of
the BEP (fold change from baseline to C2D1)

| | | PFS, mo | HR vs. Zim Mono |
|---|---|---|---|
| Biomarker, Treatment Arm | n | (95% CI) | (95% CI) |
| BEP, Zim Mono | 32 | 5.7 | — |
| | | (1.81, 9.69) | |
| BEP, Dom Combo | 86 | 11.2 | 0.59 |
| | | (7.56, 17.68) | [0.35, 0.99] |
| CXCL9 Low Induction at | 12 | 9.7 | — |
| C2D1, Zim Mono | | [3.48, NE] | |
| CXCL9 Low Induction at | 47 | 13.9 | 0.86 |
| C2D1, Dom Combo | | [9.26, NE] | [0.35, 2.12] |
| CXCL9 High Induction at | 20 | 2.7 | — |
| C2D1, Zim Mono | | [1.41, 9.66] | |
| CXCL9 High Induction at | 39 | 7.6 | 0.49 |
| C2D1, Dom Combo | | [4.01, 14.62] | [0.25, 0.95] |
| CXCL10 Low Induction at | 12 | 11.7 | — |
| C2D1, Zim Mono | | [3.48, NE] | |
| CXCL10 Low Induction at | 47 | 12 | 1.08 |
| C2D1, Dom Combo | | [7.56, NE] | [0.44, 2.65] |
| CXCL10 High Induction at | 20 | 2.7 | — |
| C2D1, Zim Mono | | [1.41, 9.66] | |
| CXCL10 High Induction at | 39 | 9.9 | 0.39 |
| C2D1, Dom Combo | | [4.01, 17.68] | [0.20, 0.77] |
| IL-12B Low Induction at | 6 | 2.5 | — |
| C2D1, Zim Mono | | [1.28, 11.73] | |
| IL-12B Low Induction at | 53 | 13.2 | 0.28 |
| C2D1, Dom Combo | | [7.56, NE] | [0.11, 0.75] |
| IL-12B High Induction at | 26 | 9.6 | — |
| C2D1, Zim Mono | | [2.69, 14.82] | |
| IL-12B High Induction at | 33 | 7.7 | 0.86 |
| C2D1, Dom Combo | | [4.01, 13.86] | [0.45, 1.66] |
| IL-18 Low Induction at | 14 | 4.5 | — |
| C2D1, Zim Mono | | [1.45, 11.73] | |
| IL-18 Low Induction at | 45 | 14.6 | 0.33 |
| C2D1, Dom Combo | | [9.26, NE] | [0.14, 0.75] |
| IL-18 High Induction at | 18 | 5.7 | — |
| C2D1, Zim Mono | | [1.48, 14.82] | |
| IL-18 High Induction at | 41 | 4.9 | 0.95 |
| C2D1, Dom Combo | | [3.55, 11.99] | [0.48, 1.89] |
| IFNγ Low Induction at | 12 | 11.7 | — |
| C2D1, Zim Mono | | [1.38, NE] | |
| IFNγ Low Induction at | 47 | 13.2 | 0.95 |
| C2D1, Dom Combo | | [7.62, NE] | [0.36, 2.49] |
| IFNγ High Induction at | 19 | 4.5 | — |
| C2D1, Zim Mono | | [1.48, 9.56] | |
| IFNγ High Induction at | 41 | 9.9 | 0.50 |
| C2D1, Dom Combo | | [4.83, NE] | [0.26, 0.96] |

BEP: biomarker evaluable population for cytokine analysis, individual numbers may vary; HR: Hazard Ratio, PFS: progression free survival; NE: not evaluable; Zim mono: zimberelimab monotherapy (See Table 11); Dom combo: domvanalimab combination (See Table 11)

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. It will be understood by one skilled in the art that the terms “>” and “greater than” are interchangeable, and that such a term is not inclusive of a value to which the term refers. Similarly, it will be understood by one skilled in the art that the terms “<,” “less than,” and “lower than” are interchangeable, and that such a term is not inclusive of a value to which the term refers. It will also be understood by one skilled in the art that the terms “≥” and “greater than or equal to” are interchangeable, and that such a term is inclusive of a value to which the term refers. Similarly, it will be understood by one skilled in the art that the terms “≤” “less than or equal to,” and “lower than or equal to” are interchangeable, and that such a term is inclusive of a value to which the term refers. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third, and upper third, etc. Finally, as will be understood by one skilled in the art, a range includes each individual member falling within the range.

All publications, patent applications, issued patents, and other references cited in this specification are herein incorporated by reference for the purpose described herein.

Other embodiments are set forth in the following claims.

What is claimed is:

1. Use of a biomarker to identify a patient for treatment with a combination therapy comprising an anti-TIGIT antibody and an anti-PD-(L)1 antibody, wherein the patient has cancer and is identified for treatment with the combination therapy when a sample obtained from the patient comprises a biomarker comprising a CD155 expression level greater than or equal to a CD155 reference level and wherein optionally the biomarker further comprises (a) a PD-L1 expression level greater than or equal to a PD-L1 reference level; (b) a CD226 expression level greater than or equal to a CD226 reference level; (c) an adenosine pathway biomarker expression level lower than an adenosine pathway biomarker reference level; (d) a CD73 expression level lower than a CD73 reference level; or (e) any combination of (a), (b), (c), and (d).

2. The use of claim 1, wherein the anti-TIGIT antibody is (i) an Fc-silent anti-TIGIT antibody, or (ii) domvanalimab.

3. The use of claim 1 or 2, wherein the combination therapy further comprises chemotherapy.

4. The use of any one of claims 1-3, wherein the cancer is (i) Stage I, Stage II, or Stage III, optionally wherein the cancer is resectable, or (ii) locally advanced or metastatic.

5. The use of any one of claims 1-4, wherein the cancer is a solid tumor.

6. The use of claim 5, wherein the cancer is selected from the group consisting of gastrointestinal cancer, genitourinary cancer, gynecological cancer, head and neck cancer, and lung cancer.

7. A method for treating cancer in a patient, comprising administering to the patient a combination therapy comprising a therapeutically effective amount of an anti-TIGIT antibody and a therapeutically effective amount of an anti-PD-(L)1 antibody when a sample obtained from the patient comprises a biomarker comprising a CD155 expression level greater than or equal to a CD155 reference level and wherein optionally the biomarker further comprises (a) a PD-L1 expression level greater than or equal to a PD-L1 reference level; (b) a CD226 expression level greater than or equal to a CD226 reference level; (c) an adenosine pathway biomarker expression level lower than an adenosine pathway biomarker reference level; (d) a CD73 expression level lower than a CD73 reference level; or (e) any combination of (a), (b), (c), and (d).

8. The method of claim 7, wherein the anti-TIGIT antibody is (i) an Fc-silent anti-TIGIT antibody, or (ii) domvanalimab.

9. The method of claim 7 or 8, wherein the combination therapy further comprises chemotherapy.

10. The method of any one of claims 7-9, wherein the cancer is (i) Stage I, Stage II, or Stage III, optionally wherein the cancer is resectable, or (ii) locally advanced or metastatic.

11. The method of any one of claims 7-10, wherein the cancer is a solid tumor.

12. The method of claim 11, wherein the cancer is selected from the group consisting of gastrointestinal cancer, genitourinary cancer, gynecological cancer, head and neck cancer, and lung cancer.

13. A method for identifying a patient with cancer for treatment with a combination therapy comprising a therapeutically effective amount of an anti-TIGIT antibody and a therapeutically effective amount of an anti-PD-(L)1 antibody, comprising:
(a) measuring in a sample obtained from a patient a level of CD155 and optionally one or more of PD-Lt, CD226, an adenosine pathway biomarker, and CD73;
(b) comparing the level measured in (a) to a respective reference level; and
(c) identifying the patient for treatment with the therapy when the measured CD155 level is greater than or equal to a CD155 reference level and optionally the measured PD-L1 level is greater than or equal to a PD-L1 reference level.

14. The method of claim 13, wherein the anti-TIGIT antibody is (i) an Fc-silent anti-TIGIT antibody, or (ii) domvanalimab.

15. The method of claim 13 or 14, wherein the combination therapy further comprises chemotherapy.

16. The method of any one of claims 13-15, wherein the cancer is Stage I, Stage II, or Stage III, optionally wherein the cancer is resectable, or (ii) locally advanced or metastatic.

17. The method of any one of claims 13-16, wherein cancer is a solid tumor.

18. The method of claim 17, wherein the cancer is selected from the group consisting of gastrointestinal cancer, genitourinary cancer, gynecological cancer, head and neck cancer, and lung cancer.

19. A method for treating a patient, comprising:
(a) measuring in a sample obtained from a patient a level of one or more of PD-L1, CD155 and optionally a biomarker that is (i) a CD226 expression level greater than or equal to a CD226 reference level; (ii) an adenosine pathway biomarker expression level lower than an adenosine pathway biomarker reference level; (iii) a CD73 expression level lower than a CD73 reference level; or (v) any combination of (i), (ii), and (iii);
(b) comparing the level of each biomarker measured in (a) to a respective reference level; and
(c) administering to the patient a combination therapy comprising a therapeutically effective amount of an anti-TIGIT antibody and a therapeutically effective amount of an anti-PD-(L)1 antibody when the measured CD155 level is greater than or equal to a CD155 reference level and optionally, when measured, the measured PD-L1 level is greater than or equal to a PD-L1 reference level.

20. The method of claim 19, wherein the anti-TIGIT antibody is (i) an Fc-silent anti-TIGIT antibody, or (ii) domvanalimab.

21. The method of claim 19 or 20, wherein the combination therapy comprises chemotherapy.

22. The method of any one of claims 19-21, wherein the patient has cancer.

23. The method of any one of claim 22, wherein the patient has a solid tumor.

24. The method of claim 23, wherein the solid tumor is selected from the group consisting of ovarian cancer, endometrial cancer, breast cancer, lung cancer, colon cancer, prostate cancer, cervical cancer, biliary cancer, pancreatic cancer, gastric cancer, esophageal cancer, liver cancer, kidney cancer, head and neck cancer, mesothelioma, melanoma, sarcomas, central nervous system (CNS) hemangioblastomas, and brain tumors.

25. The method of claim 22, wherein the patient has a cancer selected from the group consisting of gastrointestinal cancer, genitourinary cancer, gynecological cancer, head and neck cancer and lung cancer.

26. The use or method of any one of the preceding claims, wherein the sample is a sample comprising tumor cells.

27. The use or method of any one of the preceding claims, wherein the sample is a sample comprising immune cells.

28. The use or method of any one of the preceding claims, wherein the sample is a tumor biopsy.

29. The use or method of any one of the preceding claims, wherein the CD155 expression level is measured by immunohistochemistry (IHC).

30. The use or method of any one of the preceding claims, wherein the reference level is (i) a median expression level derived from samples obtained from a group of patients with the same cancer, or (ii) an amount ≤30%, ≤25%, ≤20%, ≤15%, ≤10%, or ≤5% above or below the median expression level.

31. The use or method of claim 29 or 30, wherein the expression level is measured based on IHC staining intensity and/or percentage of staining-positive cells.

32. The use or method of claim 31, wherein the staining-positive cells are tumor cells.

33. The use or method of claim 31, wherein the staining-positive cells are immune cells.

34. The use or method of claim 31, wherein the staining-positive cells are cells of the tumor microenvironment and are not immune cells or tumor cells.

35. The use or method of claim 31, wherein the staining-positive cells are (i) tumor cells and immune cells, or (i) tumor cells, immune cells and other cells of the tumor microenvironment.

36. The use or method of any one of claims 31-35, wherein the IHC staining intensity and/or the percentage of staining-positive cells is determined based on cytoplasmic staining.

37. The use or method of any one of claims 31-35, wherein the IHC staining intensity and/or the percentage of staining-positive cells is determined based on membrane staining.

38. The use or method of any one of claims 31-35, wherein the IHC staining intensity and/or the percentage of staining-positive cells is determined based on cytoplasmic staining and membrane staining.

39. The use or method of any one of claims 1-38, wherein the CD155 expression level is measured as % TC.

40. The use or method of any one of claims 1-38, wherein the CD155 expression level is measured as % IC.

41. The use or method of any one of claims 1-38, wherein the CD155 expression level is measured as an H-score.

42. The method of any one of claims 19-41, wherein the sample is PD-L1 high or is PD-L1 positive.

43. The method of claim 42, wherein the sample is PD-L1 high when a PD-L1-positive tumor cell fraction (PD-L1 % TC) of the sample is greater than or equal to 50%.

44. The method of claim 42, wherein the sample is PD-L1 high when a Tumor Area Proportion (TAP) of the sample is greater than or equal to 5%.

45. The method of claim 42, wherein the sample is PD-L1 positive when a PD-L1-positive tumor cell fraction (PD-L1 % TC) of the sample is greater than or equal to 1%.

46. The method of claim 42, wherein the sample is PD-L1 positive when a Tumor Area Proportion (TAP) of the sample is greater than or equal to 1%.

47. A method treating cancer in a patient having a CD155 high tumor, the method comprising administering to the patient a combination therapy comprising a therapeutically effective amount of a TIGIT antagonist and a therapeutically effective amount of a PD-(L)1 antagonist, wherein the CD155 high tumor has a CD155 expression level greater than or equal to (i) a median tumor H-score derived from samples obtained from a group of patients known to have the same cancer, or (ii) a value about 30%, about 25%, about 20%, about 15%, about 10%, or about 5% above or below the median tumor H-score.

48. The method of claim 47, wherein a CD155 high tumor has a CD155 expression level greater than or equal to (i) a median tumor H-score derived from samples obtained from a group of patients known to have the same cancer, or (ii) a value about 1%, 2%, 3%, 4%, 5%, 6%, 7%, 8%, 9%, 10%, 11%, 12%, 13%, 14%, 15%, 16%, 17%, 18%, 19%, or 20%, above or below the median tumor H-score.

49. A method treating cancer in a patient having a CD155 high tumor, the method comprising administering to the patient a combination therapy comprising a therapeutically effective amount of a TIGIT antagonist and a therapeutically effective amount of a PD-(L)1 antagonist, wherein a CD155 high tumor has a CD155 expression level greater than or equal to (i) a median 2+ or 3+ % TC derived from samples obtained from a group of patients known to have the same cancer, or (ii) a value about 30%, about 25%, about 20%, about 15%, about 10%, or about 5% above or below the median 2+ or 3+ % TC.

50. The method of claim 49, wherein a CD155 high tumor has a CD155 expression level greater than or equal to (i) a median 2+ or 3+ % TC derived from samples obtained from a group of patients known to have the same cancer, or (ii) a value about 1%, 2%, 3%, 4%, 5%, 6%, 7%, 8%, 9%, 10%, 11%, 12%, 13%, 14%, 15%, 16%%, 17%, 18%, 19%, or 20% above or below the median 2+ or 3+ % TC.

51. A method treating cancer in a patient having a CD155 high tumor, the method comprising administering to the patient a combination therapy comprising a therapeutically effective amount of a TIGIT antagonist and a therapeutically effective amount of a PD-(L)1 antagonist, wherein a CD155 high tumor has a CD155 tumor H-score ≥95, ≥100, ≥105, ≥110, ≥115, ≥120, ≥125, ≥130, ≥135, ≥140, ≥145, ≥150, ≥155, ≥160, ≥165, ≥170, or ≥175.

52. The method of claim 51, wherein a CD155 high tumor has a CD155 tumor H-score ≥110, ≥115, ≥120, ≥125, ≥130, ≥135, ≥140, ≥145, ≥150, ≥155, or ≥160.

53. A method treating cancer in a patient having a CD155 high tumor, the method comprising administering to the patient a combination therapy comprising a therapeutically effective amount of a TIGIT antagonist and a therapeutically effective amount of a PD-(L)1 antagonist, wherein a CD155 high tumor has a CD155 2+ or 3+ % TC ≥30%, ≥35%, ≥40%, ≥45%, ≥50%, ≥55%, ≥60%, or ≥65%.

54. The method of claim 53, wherein a CD155 high tumor has a CD155 2+ or 3+ % TC≥30%, ≥35%, ≥40%, ≥45%, or ≥50%.

55. The method of any one of claims 47-54, wherein the TIGIT antagonist is an anti-TIGIT antibody, the PD-(L) antagonist is an anti-PD-(L)1 antibody, or the TIGIT antagonist is an anti-TIGIT antibody and the PD-(L) antagonist is an anti-PD-(L)1 antibody.

56. The method of 55, wherein the anti-TIGIT antibody is (i) an Fc-silent anti-TIGIT antibody, or (ii) domvanalimab.

57. A method treating cancer in a patient having a CD155 high tumor, the method comprising administering to the patient a combination therapy comprising a therapeutically effective amount of an anti-TIGIT antibody that is Fc-silent and a therapeutically effective amount of a PD-(L)1 antagonist, wherein a CD155 high tumor has a CD155 expression level greater than or equal to a CD155 reference level.

58. The method of claim 57, wherein the CD155 reference level is
(a) a median tumor H-score derived from samples obtained from a group of patients known to have the same cancer, or a value about 30%, about 25%, about 20%, about 15%, about 10%, or about 5% above or below the median tumor H-score; or
(b) a median 2+ or 3+ % TC derived from samples obtained from a group of patients known to have the same cancer, or a value about 30%, about 25%, about 20%, about 15%, about 10%, or about 5% above or below the median 2+ or 3+ % TC; or
(c) a tumor H-score ≥95, ≥100, ≥105, ≥110, ≥115, ≥120, ≥125, ≥130, ≥135, ≥140, ≥145, ≥150, ≥155, ≥160, ≥165, ≥170, or ≥175; or
(d) a 2+ or 3+ % TC ≥30%, ≥35%, ≥40%, ≥45%, ≥50%, ≥55%, ≥60%, or ≥65%.

59. The method of claim 57, wherein the CD155 reference level is (a) a median tumor H-score derived from samples obtained from a group of patients known to have the same cancer, or a value about 1%, 2%, 3%, 4%, 5%, 6%, 7%, 8%, 9%, 10%, 11%, 12%, 13%, 14%, 15%, 16%, 17%, 18%, 19%, or 20%, above or below the median tumor H-score; or
(b) a median 2+ or 3+ % TC derived from samples obtained from a group of patients known to have the same cancer, or a value about 1%, 2%, 3%, 4%, 5%, 6%, 7%, 8%, 9%, 10%, 11%, 12%, 13%, 14%, 15%, 16%%, 17%, 18%, 19%, or 20% above or below the median 2+ or 3+ % TC;
(c) a tumor H-score ≥110, ≥115, ≥120, ≥125, ≥130, ≥135, ≥140, ≥145, ≥150, ≥155, or ≥160; or
(d) a 2+ or 3+ % TC ≥30%, ≥35%, ≥40%, ≥45%, or ≥50%.

60. The method of any one of claims 47-59, wherein the PD-(L) antagonist is an anti-PD-(L)1 antibody, optionally wherein the PD-(L)1 antibody is atezolizumab, avelumab, cemiplimab, dostarlimab, durvalumab, nivolumab, pembrolizumab, retifanlimab, tislelizumab, toripalimab, or zimberelimab.

61. The method of any one of claims 47-60, wherein the CD155 high tumor is PD-L1 positive, optionally wherein PD-L1 positive is a PD-L1-positive tumor cell fraction (PD-L1 % TC) greater than or equal to 1%, or a PD-L1 Tumor Area Proportion (TAP) greater than or equal to 1%, or a PD-L1 CPS score ≥1.

62. The method of any one of claims 47-60, wherein the PD-L1 expression level in the CD155 high tumor is ≥10% TC.

63. The method of any one of claim 62, wherein the PD-L1 expression level in the CD155 high tumor is ≥10% TC.

64. The method of any one of claim 62, wherein the PD-L1 expression level in the CD155 high tumor is ≥50% TC.

65. The method of any one of claims 47-60, wherein the PD-L1 expression level in the CD155 high tumor is ≥5% TAP or CPS ≥5.

66. The method of any one of claim 65, wherein the PD-L1 expression level in the CD155 high tumor is ≥10% TAP or CPS ≥10.

67. A method treating cancer in a patient having a CD155 high tumor, the method comprising administering to the patient a combination therapy comprising a therapeutically effective amount of an anti-TIGIT antagonist and a therapeutically effective amount of a PD-(L)1 antagonist, wherein a CD155 high tumor has a CD155 expression level greater than or equal to a CD155 reference level and wherein the PD-L1 expression level in the CD155 high tumor is ≥10% TC, ≥5% TAP, or ≥5% CPS.

68. The method of claim 67, wherein the PD-L1 expression level in the CD155 high tumor is ≥10% TAP or CPS ≥10.

69. The method of claim 67, wherein the PD-L1 expression level in the CD155 high tumor is ≥50% TC.

70. The method of any one of claims 67-69, wherein the CD155 reference level is
(a) a median tumor H-score derived from samples obtained from a group of patients known to have the same cancer, or a value about 30%, about 25%, about 20%, about 15%, about 10%, or about 5% above or below the median tumor H-score; or
(b) a median 2+ or 3+ % TC derived from samples obtained from a group of patients known to have the same cancer, or a value about 30%, about 25%, about 20%, about 15%, about 10%, or about 5% above or below the median 2+ or 3+ % TC; or
(c) a tumor H-score ≥95, ≥100, ≥105, ≥110, ≥115, ≥120, ≥125, ≥130, ≥135, ≥140, ≥145, ≥150, ≥155, ≥160, ≥165, ≥170, or ≥175; or
(d) a 2+ or 3+ % TC ≥30%, ≥35%, ≥40%, ≥45%, ≥50%, ≥55%, ≥60%, or ≥65%.

71. The method of any one of claims 67-69, wherein the CD155 reference level is
(a) a median tumor H-score derived from samples obtained from a group of patients known to have the same cancer, or a value about 1%, 2%, 3%, 4%, 5%, 6%, 7%, 8%, 9%, 10%, 11%, 12%, 13%, 14%, 15%, 16%, 17%, 18%, 19%, or 20%, above or below the median tumor H-score; or
(b) a median 2+ or 3+ % TC derived from samples obtained from a group of patients known to have the same cancer, or a value about 1%, 2%, 3%, 4%, 5%, 6%, 7%, 8%, 9%, 10%, 11%, 12%, 13%, 14%, 15%, 16%%, 17%, 18%, 19%, or 20% above or below the median 2+ or 3+ % TC;
(c) a tumor H-score ≥110, ≥115, ≥120, ≥125, ≥130, ≥135, ≥140, ≥145, ≥150, ≥155, or ≥160; or
(d) a 2+ or 3+ % TC ≥30%, ≥35%, ≥40%, ≥45%, or ≥50%.

72. The method of any one of claims 67-71, wherein the TIGIT antagonist is an anti-TIGIT antibody, the PD-(L) antagonist is an anti-PD-(L)1 antibody, or the TIGIT antagonist is an anti-TIGIT antibody and the PD-(L) antagonist is an anti-PD-(L)1 antibody.

73. The method of 72, wherein the anti-TIGIT antibody is (i) an Fe-silent anti-TIGIT antibody, or (ii) domvanalimab.

74. The method of claim 72, wherein the anti-PD-(L)1 antibody is atezolizumab, avelumab, cemiplimab, dostarlimab, durvalumab, nivolumab, pembrolizumab, retifanlimab, tislelizumab, toripalimab, or zimberelimab.

75. The method of any one of claim 47-54, 58, 59, 70, or 71, wherein CD155 staining intensity and percentage of staining-positive cells is determined based on cytoplasmic staining and membrane staining.

76. The method of any one of claim 47-54, 58, 59, 70, or 71, wherein CD155 staining intensity and percentage of staining-positive cells is determined based on membrane staining.

77. A method treating cancer in a patient having high expression levels of CXCL9, CXCL10, IL-6 or IFNγ before treatment, the method comprising administering to the patient a combination therapy comprising a therapeutically effective amount of a TIGIT antagonist and a therapeutically effective amount of a PD-(L)1 antagonist, wherein a high expression level of CXCL9, CXCL10, IL-6 or IFNγ is an amount greater than or equal to a reference value.

78. The method of claim 77, wherein the reference value is the CD155 reference level is median value derived from samples obtained from a group of patients known to have the same cancer, or a value about 30%, about 25%, about 20%, about 15%, about 10%, or about 5% above or below the median.

79. The method of claim 77 or 78, wherein the TIGIT antagonist is an anti-TIGIT antibody, the PD-(L) antagonist is an anti-PD-(L)1 antibody, or the TIGIT antagonist is an anti-TIGIT antibody and the PD-(L) antagonist is an anti-PD-(L)1 antibody.

80. The method of 79, wherein the anti-TIGIT antibody is (i) an Fc-silent anti-TIGIT antibody, or (ii) domvanalimab.

81. The method of claim 79, wherein the anti-PD-(L)1 antibody is atezolizumab, avelumab, cemiplimab, dostarlimab, durvalumab, nivolumab, pembrolizumab, retifanlimab, tislelizumab, toripalimab, or zimberelimab.

82. The method of any one of claims 47-81, wherein the combination therapy further comprises chemotherapy.

83. The method of any one of claims 47-82, wherein the cancer is (i) Stage I, Stage II, or Stage III.

84. The method of claim 78, wherein the cancer is resectable and the combination therapy is administered before surgery (neoadjuvant treatment), after surgery (adjuvant treatment), or before and after surgery (neoadjuvant plus adjuvant treatment).

85. The method of any one of claims 47-81, wherein the cancer is locally advanced or metastatic.

86. The method of claim 85, wherein the cancer is resectable and the combination therapy is administered before surgery (neoadjuvant treatment), after surgery (adjuvant treatment), or before and after surgery (neoadjuvant plus adjuvant treatment).

87. The method of claim 85, wherein the locally advanced or metastatic cancer is previously untreated with the proviso that prior surgery is allowed.

88. The method of claim 85, wherein the locally advanced or metastatic cancer is previously untreated with chemotherapy, optionally a chemotherapy comprising a platinum agent.

89. The method of claim 85, wherein the locally advanced or metastatic cancer was previously treated with chemotherapy, optionally a chemotherapy comprising a platinum agent.

90. The method of any one of claim 85, 87, 88 or 89, wherein the locally advanced or metastatic cancer is previously untreated with a PD-(L)1 antagonist, optionally an anti-PD-(L)1 antibody.

91. The method of any one of claim 86, 87, 88 or 89, wherein the locally advanced or metastatic cancer was previously treated with a PD-(L)1 antagonist, optionally an anti-PD-(L)1 antibody.

92. The method of any one of claims 47-91, wherein the cancer is a solid tumor.

93. The method of claim 92, wherein the cancer is selected from the group consisting of gastrointestinal cancer, genitourinary cancer, gynecological cancer, head and neck cancer, and lung cancer.

94. The method of claim 93, wherein the cancer is lung cancer or upper gastrointestinal (GI) cancer.

95. The method of claim 94, wherein the cancer is squamous cell lung carcinoma, non-squamous cell lung carcinoma, gastric cancer, gastroesophageal junction (GEJ) cancer, esophageal adenocarcinoma (EAC), or esophageal squamous cell carcinoma (ESCC).

96. The method of claim 94, wherein the cancer is non-squamous cell lung carcinoma (NSCLC), gastric cancer, gastroesophageal junction (GEJ) cancer, or esophageal adenocarcinoma (EAC).

97. The method of claim 92, wherein the cancer is selected from the group consisting of bladder cancer, breast cancer, endometrial cancer, head and neck squamous cell carcinoma, melanoma, ovarian cancer, and renal cell carcinoma.

98. The method of claim 92, wherein the cancer is selected from the group consisting of melanoma, non-small cell lung cancer (NSCLC), small cell lung cancer (SCLC), head and neck squamous cell cancer (HNSCC), classical Hodgkin lymphoma (cHL), primary mediastinal large B-cell lymphoma (PMBCL), urothelial carcinoma, microsatellite instability-high or mismatch repair deficient cancer, microsatellite instability-high or mismatch repair deficient colorectal cancer (CRC), gastric cancer, esophageal cancer, cervical cancer, hepatocellular carcinoma (HCC), Merkel cell carcinoma (MCC), renal cell carcinoma (RCC), endometrial carcinoma, tumor mutational burden-high (TMB-H) cancer, cutaneous squamous cell carcinoma (cSCC), and triple-negative breast cancer (TNBC).
